(19) United States
(12) Patent Application Publication
(10) Pub. No.: US 2010/0170006 A1
(43) Pub. Date: Jul. 1, 2010

(54) METHODS FOR SCREENING OF NOVEL FUNCTIONS OF RECEPTOR LIKE KINASES

(75) Inventors: Zhenbiao Yang, Riverside, CA (US); Stephen Karr, Camarillo, CA (US)

Correspondence Address: Joseph R. Baker, APC;Gavrilovich, Dodd & Lindsey LLP, 4660 La Jolla Village Drive, Suite 750, San Diego, CA, 92122, US

(73) Assignee: THE REGENTS OF THE UNIVERSITY OF CALIFORNIA, Oakland, CA (US)

(21) Appl. No.: 12/641,641

(22) Filed: Dec. 18, 2009

Related U.S. Application Data
(60) Provisional application No. 61/138,902, filed on Dec. 18, 2008.

(51) Int. Cl. A01H 1/00; A01H 5/00; C12N 15/63; C40B 40/06; C40B 50/00; C12N 5/04; C12Q 1/02; C40B 40/02

(52) U.S. Cl. 800/279; 800/278; 800/298; 800/301; 435/320.1; 506/16; 506/23; 435/419; 435/29; 506/14

(57) ABSTRACT

The disclosure relates to methods for modulating plant growth and organogenesis using dominant-negative receptor-like kinases.

### CROSS REFERENCE TO RELATED APPLICATIONS

This application claims priority under 35 U.S.C. §119 from Provisional Application Ser. No. 61/138,902, filed Dec. 18, 2008, the disclosure of which is incorporated herein by reference.

### TECHNICAL FIELD

The disclosure relates to methods for modulating plant growth and organogenesis using dominant-negative receptor-like kinases.

### BACKGROUND

Receptor-like kinases (RLKs) form a large monophyletic gene family of approximately 600 members in plants (Shiu and Bleecker, Plant receptor-like kinase gene family: diversity, function and signaling. Science STKE, re22, 2001; and Shiu and Bleecker, Receptor-like kinases from Arabidopsis form a monophyletic gene family related to animal receptor kinases. Proceeding of the National Academy of Science U.S.A. 98:10763-10768, 2001). They consist of proteins that contain a single extracellular domain that is thought to be the site of ligand binding, connected to a single kinase domain, via a single transmembrane domain. Upon ligand binding the kinase domain is capable of generating a phosphorylation signaling cascade. Because of the sheer size of this gene family and of the potential functional redundancy among closely related gene family members, not much is known about the function of many of these important signaling genes. What little that was known shows that RLKs have many diverse roles in plants such as, hormone perception, plant defense, plant development and cell growth.

### SUMMARY

The disclosure provides a method of identifying the function of receptor-like kinases (RLKs) that modulate plant function and morphology comprising: identifying a family of RLKs that comprise at least 50% sequence identity in the extracellular and transmembrane domains; using a set of PCR primer pair, generating from a cDNA library of RLKs a plurality of RLKs lacking a functional kinase domain (DN-RLKs); cloning the DN-RLKs into a plant species to obtain recombinant plants comprising at least one DN-RLK from the plurality of DN-RLKs; expressing the DN-RLKs; and identifying recombinant plants having morphological or functional traits different than a wild-type plant species. In one embodiment, the family of RLKs has at least 60%, 70%, 80%, 90%, 95%, 98%, or 99% identity between members of the family. In another embodiment, the PCR primer pair comprise a first primer comprises a sequence corresponding to the extracellular domain end of the coding sequence and the second primer comprises a sequence that truncates the kinase domain or induces a mutation in the kinase domain that results in a domain lacks kinase activity. The plant species can be any plant species including crop plants. In one embodiment the plant species is Arabidopsis sp.

The disclosure also provides transgenic plants generated by the methods of the disclosure. In one embodiment, the transgenic plant comprises improved growth characteristics, pathogen resistance, plant height or metabolic activity compared to a wild-type plant.

The disclosure also provides a method of generating a transgene comprising a dominant-negative receptor-like kinases (RLKs) that modulate plant function and morphology comprising: identifying a family of RLKs that comprise at least 50% sequence identity in the extracellular and transmembrane domains; using a set of PCR primer pair, generating from a cDNA library of RLKs a plurality of RLKs lacking a functional kinase domain (DN-RLKs); cloning at least one DN-RLK from the plurality of DN-RLKs into a vector.

The disclosure also provides a method for modulating plant height, organ shape, metabolism, growth characteristics or pathogen resistance comprising the step of expressing a transgene of the disclosure in a plant, wherein the transgene encodes a receptor-like kinase (RLK) protein lacking an active receptor domain or kinase domain and wherein expression of the transgene modulates plant height, organ shape, metabolism, growth characteristics or pathogen resistance.

The disclosure also provides a method for enhancing the plant height, organ shape, metabolism, growth characteristics or pathogen resistance of a plant, comprising the steps of: (a) introducing a transgene of the disclosure into a plant, wherein the transgene encodes a receptor-like kinase protein lacking an active receptor domain or kinase domain and wherein expression of the transgene enhances the plant height, organ shape, metabolism, growth characteristics or pathogen resistance of the crop plant; and (b) growing the transgenic plant under conditions in which the transgene is expressed to enhance the plant height, organ shape, metabolism, growth characteristics or pathogen resistance of the plant.

The disclosure also provides a library of dominant-negative RLK-encoding polynucleotides wherein the polynucleotide encodes a dominant-negative RLK lacking a receptor domain or kinase domain, the library obtained by the method of the disclosure. In one embodiment the library comprise an RLK having at least 90%, 95%, 98%, 99% or 100% identity to a sequence found in the AGI accession number of Table 1.

The disclosure also provides a method of making a library of dominant-negative RLK encoding polynucleotides comprising: (a) identifying a family of RLKs having at least 50% identity to one another; (b) mutating the RLKs having identity to disrupt function ligand binding function or kinase function; and (c) cloning the mutant RLKs. The method can further comprise transforming plant cells with the mutant RLKs, growing the cells and identifying desirable phenotypes.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-J shows distance mapping tree of the extracellular domains of all receptor-like kinases (RLKs) in Arabidposis thaliana.

FIG. 2 Examination of partial distance map for the wall-associated kinase family 1.5 showing nearest neighbor protein identities. 50% was used for the cutoff point.

FIG. 3 Model of dominant negative (DN) receptor-like kinase action in vivo.

FIGS. 4A-B shows a flow chart and demonstration. A) Flowchart of gene expression database directed experiment design for DNRLKs. B) Actual demonstration of using Genevestigator gene expression data for programmed cell death (PCD) to examine senescence phenotype of DN-1.5-11 (DNWAKL14).

FIGS. 5A-F shows root and seedling growth. A-C) Examination of root hairs from 7-day old seedlings grown on MS media. A) WT, B) DN-1.12-23 (At5g01890) showing root hair branching, and C) SALK_{—}053567C (At3g28040) homozygous line for 1.12-23 subfamily member showing similar branched root hair phenotype. D-F) UV-confocal microscope images of 3-day old dark grown hypocotyls grown on MS media without supplemented sucrose. D) WT, E) DN-1.1-4 (At3g14350) showing block-like epidermal cells, and F) SALK_{—}077702 (At1g53730) showing enhanced block-like epidermal cells.

### DETAILED DESCRIPTION

As used herein and in the appended claims, the singular forms “a,” “and,” and “the” include plural referents unless the context clearly dictates otherwise. Thus, for example, reference to “a cell” includes a plurality of such cells and reference to “the gene” includes reference to one or more genes and equivalents thereof, and so forth.

Unless defined otherwise, all technical and scientific terms used herein have the same meaning as commonly understood to one of ordinary skill in the art to which this disclosure belongs. Although any methods and reagents similar or equivalent to those described herein can be used in the practice of the disclosed methods and compositions, the exemplary methods and materials are now described.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

All publications mentioned herein are incorporated herein by reference in full for the purpose of describing and disclosing the methodologies, which are described in the publications, which might be used in connection with the description herein. The publications discussed above and throughout the text are provided solely for their disclosure prior to the filing date of the present application. Nothing herein is to be construed as an admission that the inventors are not entitled to antedate such disclosure by virtue of prior disclosure. The disclosures of International Application No. PCT/US09/65766, filed Nov. 24, 2009, and International Application No. PCT/US09/65777, filed Nov. 24, 2009, are incorporated herein by reference in their entirety.

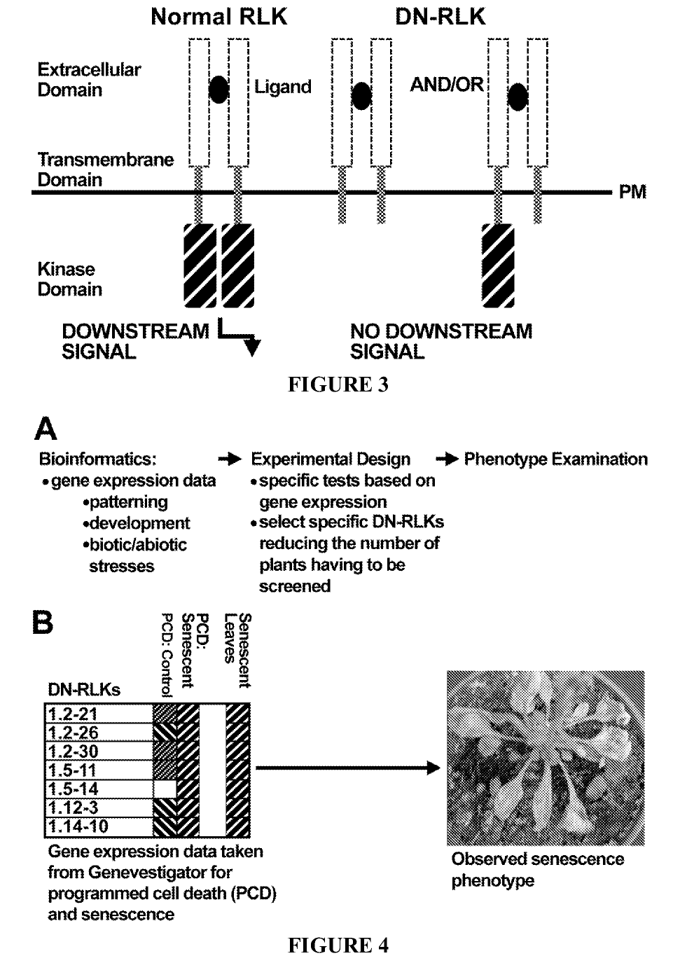

There are over 400 receptor-like kinases (RLKs) in Arabidposis that have predicted transmembrane domains and extracellular domains larger than 100 amino acids, for many of which the function is unknown or unclear. In order to better understand the functions of these RLKs the disclosure provides an approach whereby kinase-free versions of the RLKs (i.e., the dominant negative: DN) were generated and over-expressed in Arabidposis and subsequent changes in phenotypes were examined (Shpak et al., Dominant-negative receptor uncovers redundancy in the Arabidposis ERECTA leucine-rich repeat receptor-like kinase signaling pathway that regulates organ shape. The Plant Cell, 15:1095-1110, 2003). This approach works in two ways. One, the kinase free RLK may homo- or heterodimerize with the endogenous RLKs and the result would be a termination of the phosphorylation cascade, or secondly it could compete for and bind up ligand(s) that are required for signaling of the endogenous RLKs and again diminish any downstream signaling (see, e.g., FIG. 3). To date, 100 kinase free RLK constructs have been generated and 72 of these stably transformed into Arabidposis as homozygous lines. This covers over 63% of all the RLKs in kinase-free (DN) constructs and over 45% coverage in homozygous lines. These homozygous lines were then investigated for morphological, developmental and stress response phenotypes.

The dominant negative (DN) approach described herein can be used to study many different classes of receptor-like kinases in Arabidposis. This approach has allowed for the investigation of many important functions of RLKs such as nutrient sensing and response to abiotic stress. The disclosure demonstrates that the dominant negative effect shown in LRR-RLKs was not limited to just this family of RLKs but appears to work in the other classes as well.

A method of the disclosure provides a method of identifying the function of receptor-like kinases (RLKs) that modulate plant function and morphology comprising identifying a family of RLKs that comprise at least 50% sequence identity in the extracellular and transmembrane domains; using a set of PCR primer pair, generating from a cDNA library of RLKs a plurality of RLKs lacking a functional kinase domain (DN-RLKs); cloning the DN-RLKs into a plant species to obtain recombinant plants comprising at least one DN-RLK from the plurality of DN-RLKs; expressing the DN-RLKs; and identifying recombinant plants having morphological or functional traits different than a wild-type plant species. In one embodiment, the family of RLKs has at least 60%, 70%, 80%, 90%, 95%, 98%, or 99% identity between members of the family. In another embodiment, the PCR primer pair comprise a first primer comprises a sequence corresponding to the extracellular domain end of the coding sequence and the second primer comprises a sequence that truncates the kinase domain or induces a mutation in the kinase domain that results in a domain lacks kinase activity. The plant species can be any plant species including crop plants. In one embodiment the plant species is Arabidposis sp.

As described more fully below, percent identity and alignment can be performed using commercially and generally available sequence algorithms. The percent identity can be modified to range from 50% to more than 99% (and any value there between). As set forth in Table 1 a large number of sequences are available in general databases related to RLKs. These sequences can be utilized from such databases, screened and categorized into families using the percent identity. Typically the identity of the extracellular and transmembrane domains are used as a criteria for identifying a family member; however, the criteria can use one or the other or both such domain and may further include the kinase domain.

Once a family is characterized a set of primers can be designed based upon the sequences having identity across all family member or which utilize a set of degenerate primers having a degree of identity. One primer will have identity to the coding sequences of the extracellular domain (e.g., proximal or equal to the terminal end) and the other primer will have identity to the transmembrane domain or kinase domain, such that amplification of the primer pair by PCR techniques will generate a product having the extracellular and transmembrane domain, but may be lacking a kinase domain or may have induced mutation to generate a non-functional kinase domain such that the amplified product comprises a dominant-negative RLK (DN-RLK) polynucleotide encoding a DN-RLK polypeptide. The DN-RLK polynucleotide can then be cloned into a suitable vector for expression in a desired plant cell or cell type.

The vector can then be used to transform a plant cell of interest to generate a transgenic plant. Expression of the vector can be measured using various techniques as described more fully below. The function of the expressed DN-RLK can be detected by functional, phenotypical and morphological changes in the transgenic plant compared to a wild-type plant.

By comparing the DN-RLK and knockout lines confirmed that the DN-RLK was responsible for the observed phenotype, which was stronger than the knockout. This was also the case with the DN-ERECTA mutant in Shpak et al., where they observed a similar phenotype to the ERECTA knockout (Shpak et al., 2003, supra). They also showed that there was functional redundancy of the ERECTA receptor by expressing the DN in an ERECTA knockout, this phenotype was more severe than the single mutant suggesting that the DN was interfering with ERECTA-like receptors and adverting functional redundancy problems. The disclosure further demonstrates that the most common morphological phenotypes when grown on soil affected the leaf size and shape and an increase in the time it took for the plants to flower. It is also important to note that under normal conditions the majority (76.4%) of the DN-RLKs showed no detectable phenotype. This was a logical observation as RLKs may function in many diverse ways: development, pathogen response, light response or nutrient response, to name just a few and under normal conditions these RLKs may not be expressed or necessary until a cue elicits their action. The disclosure provides sensitizing screens and bioinformatics that allowed for the discovery of novel phenotypes. The DN-RLK provided by the disclosure is an excellent resource for future investigations of receptor-like kinase functions in Arabidposis as well as agronomically important species like rice or corn.

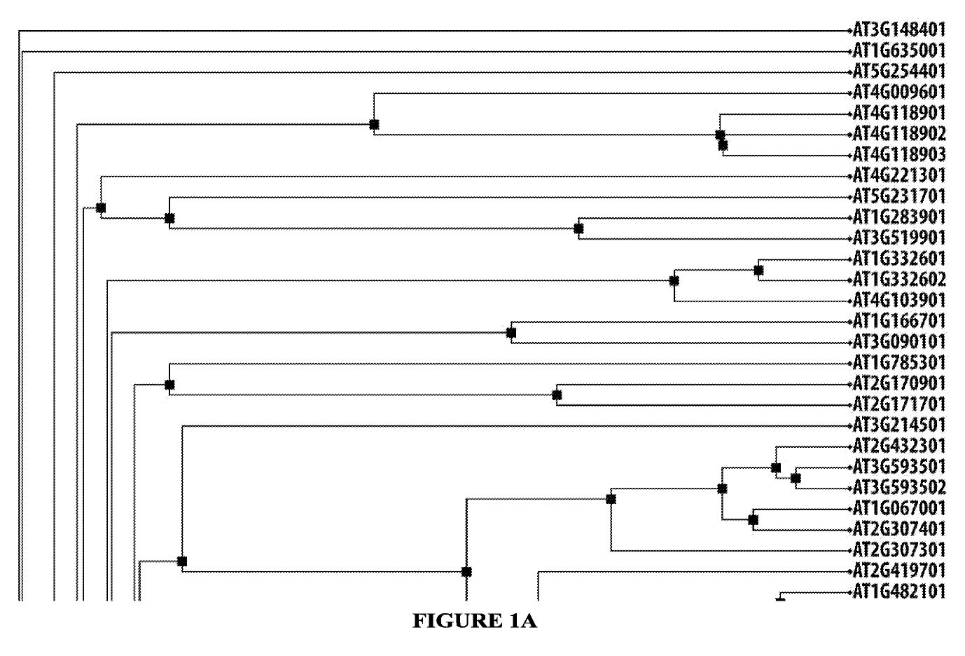

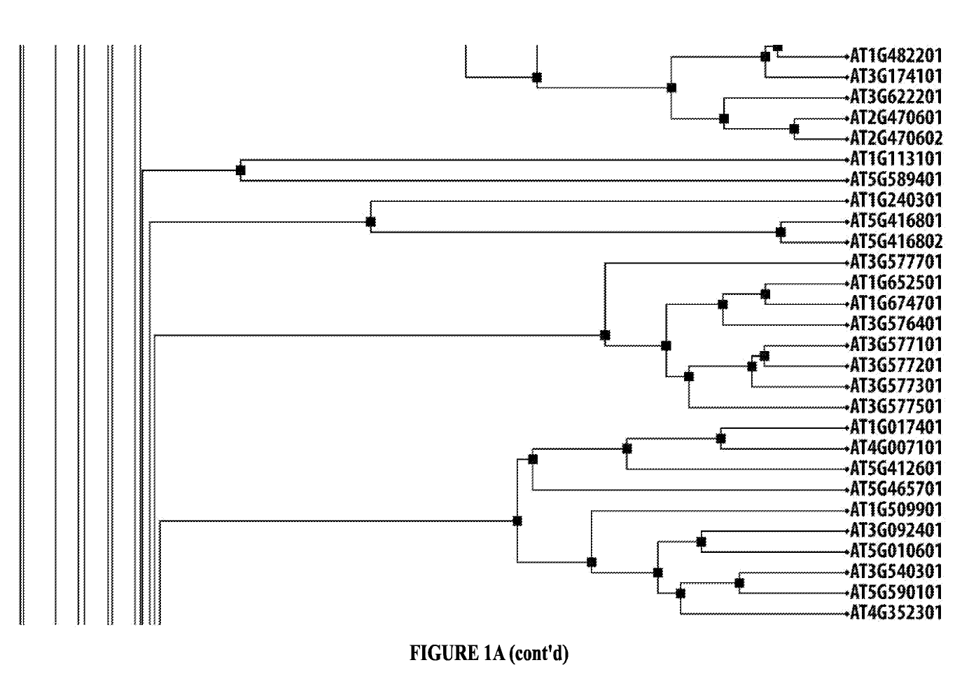

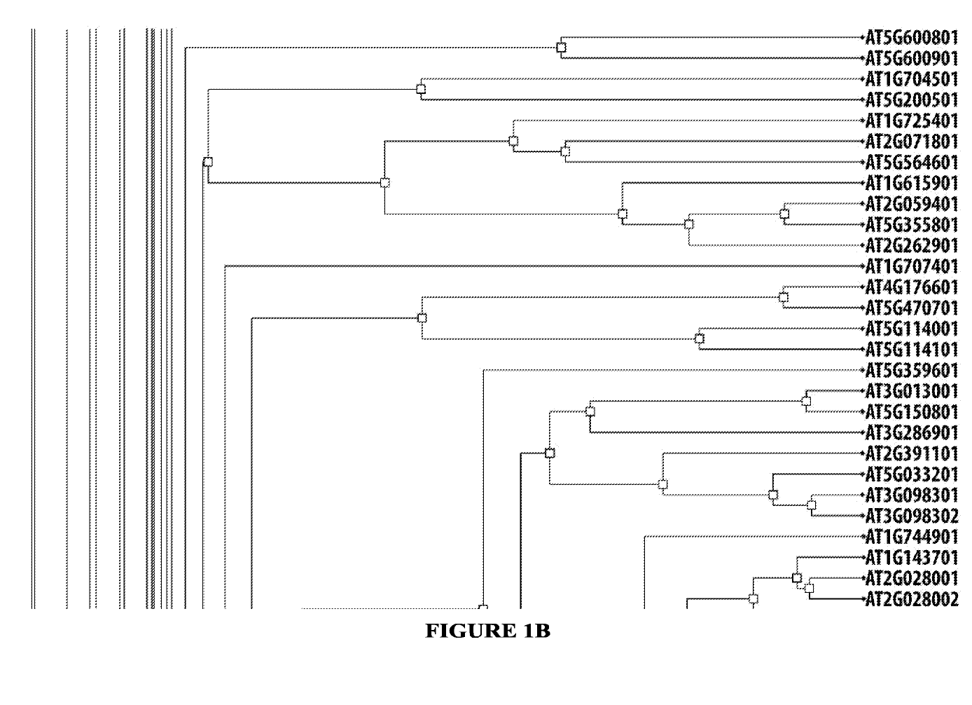

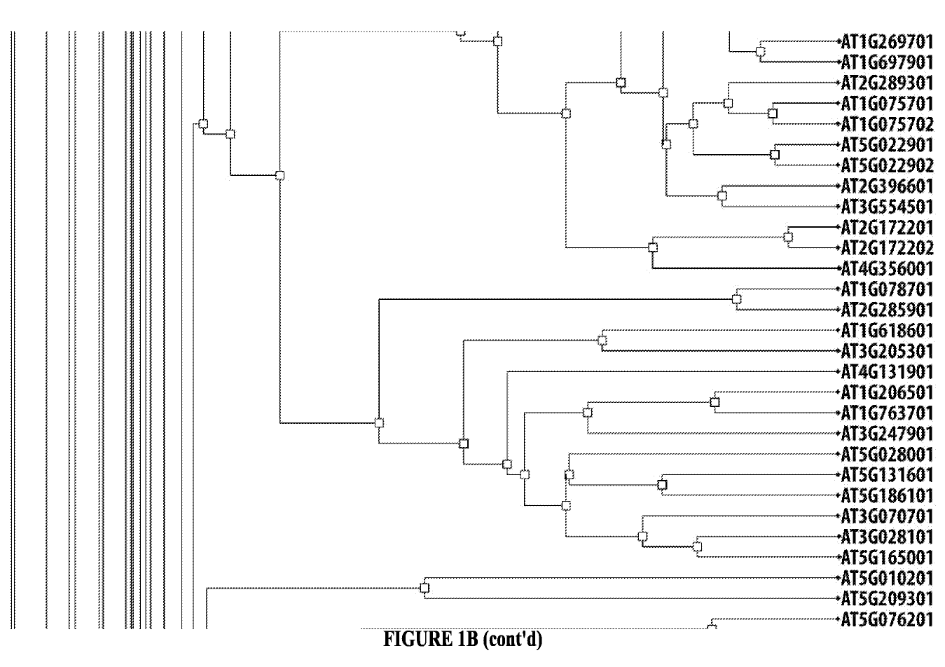

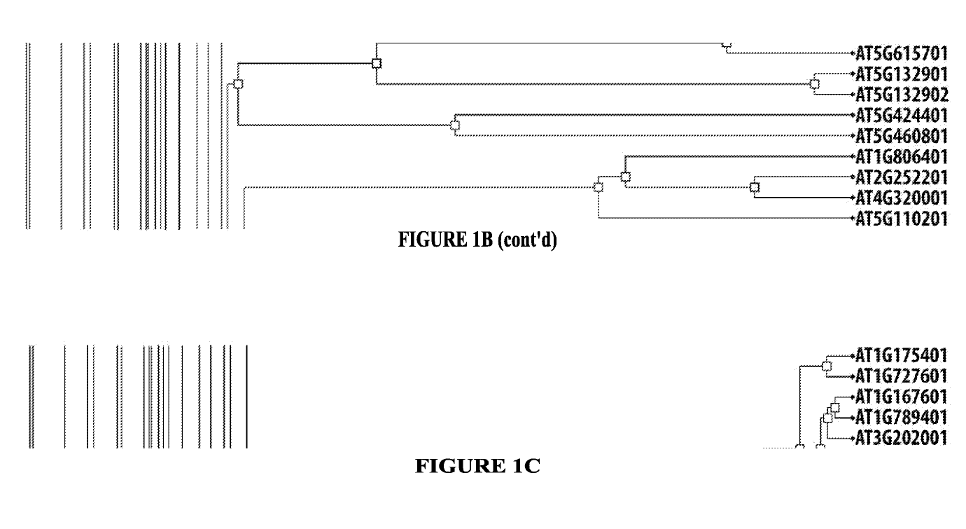

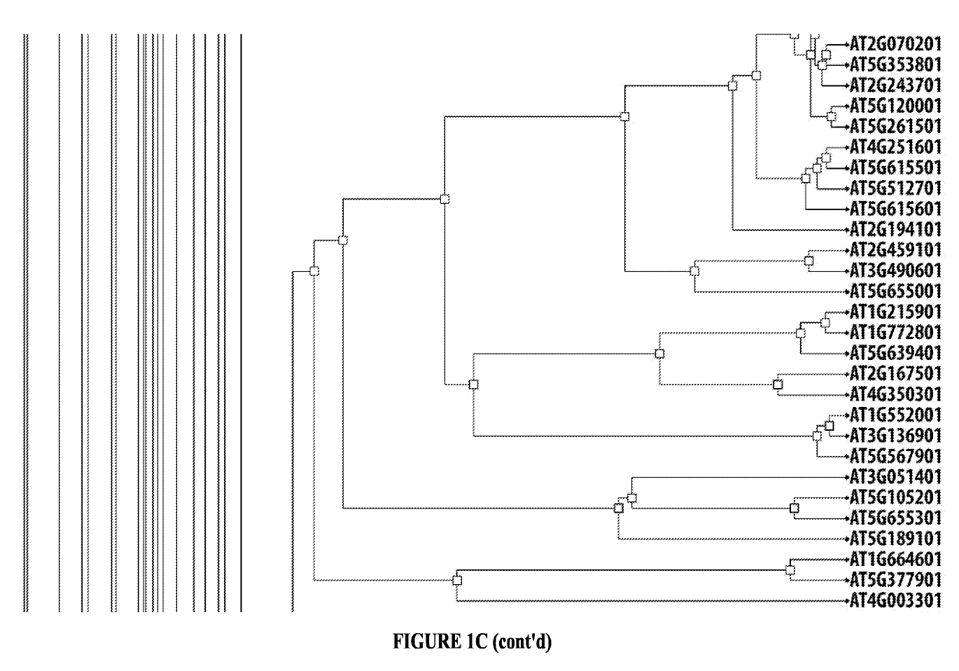

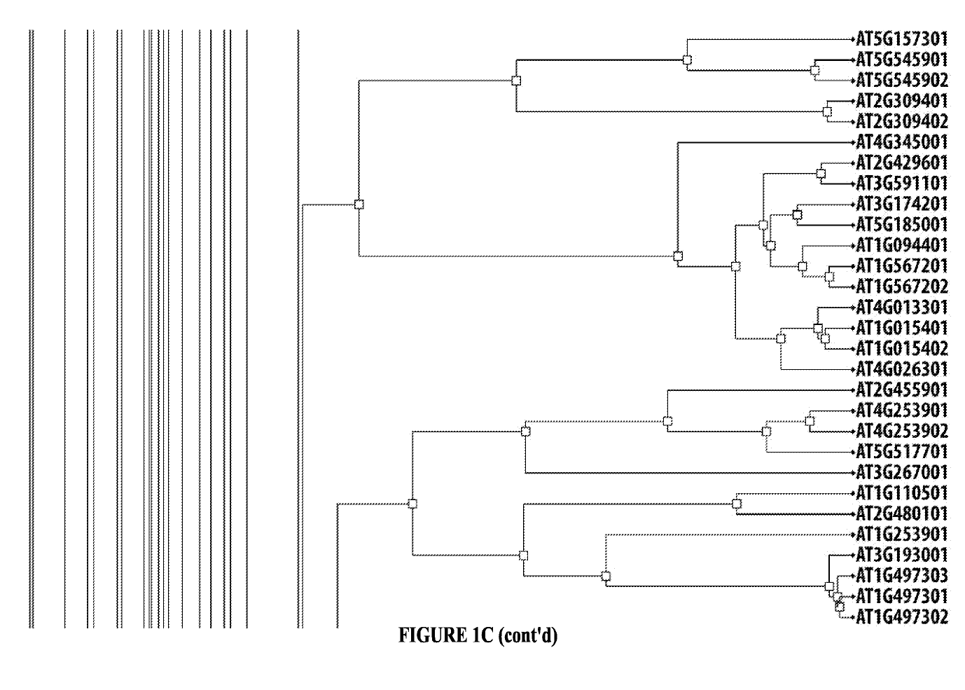

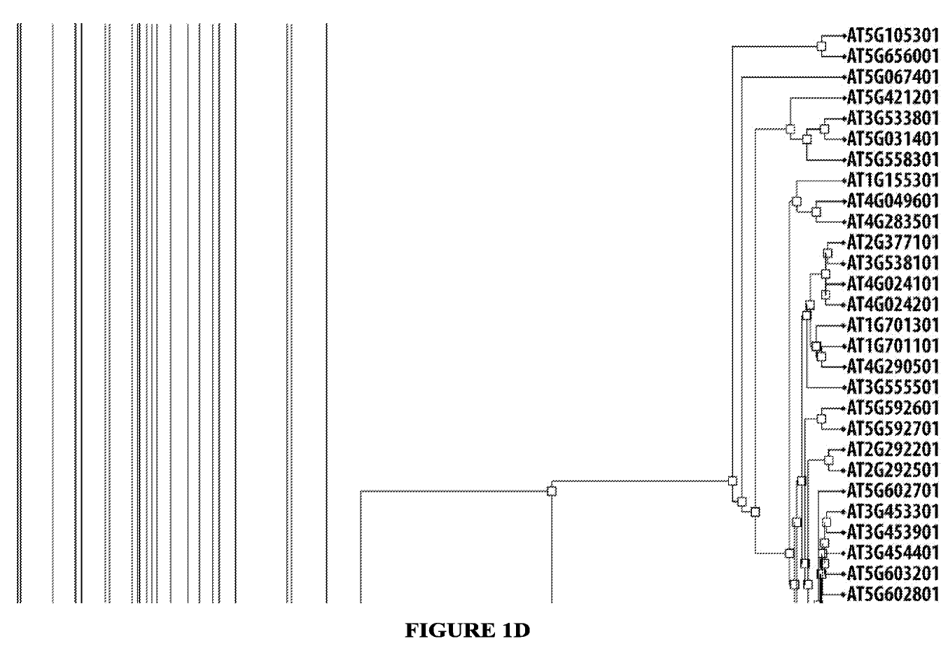

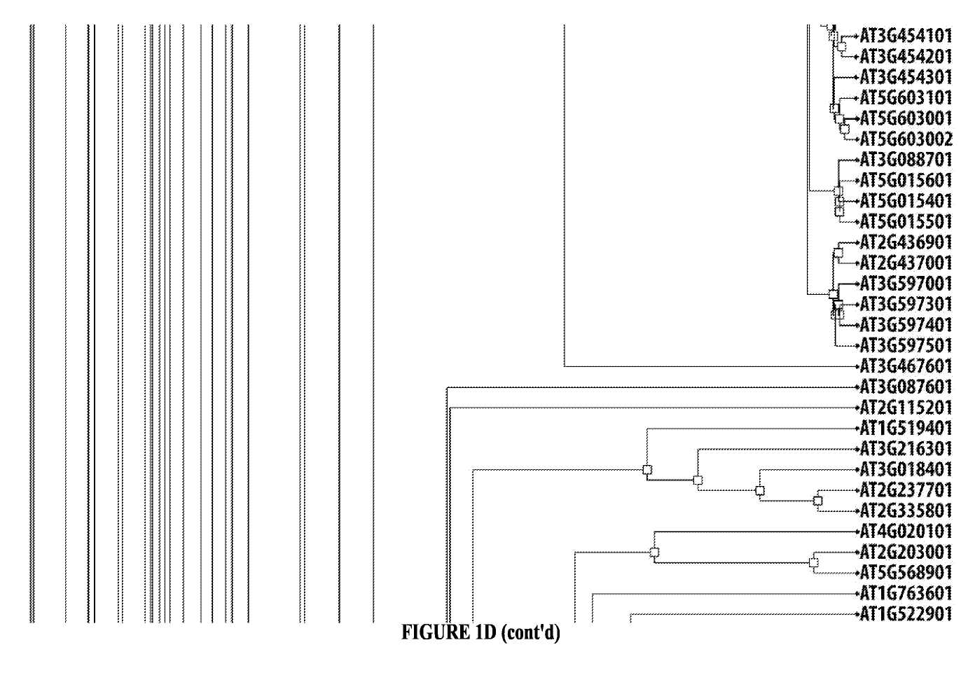

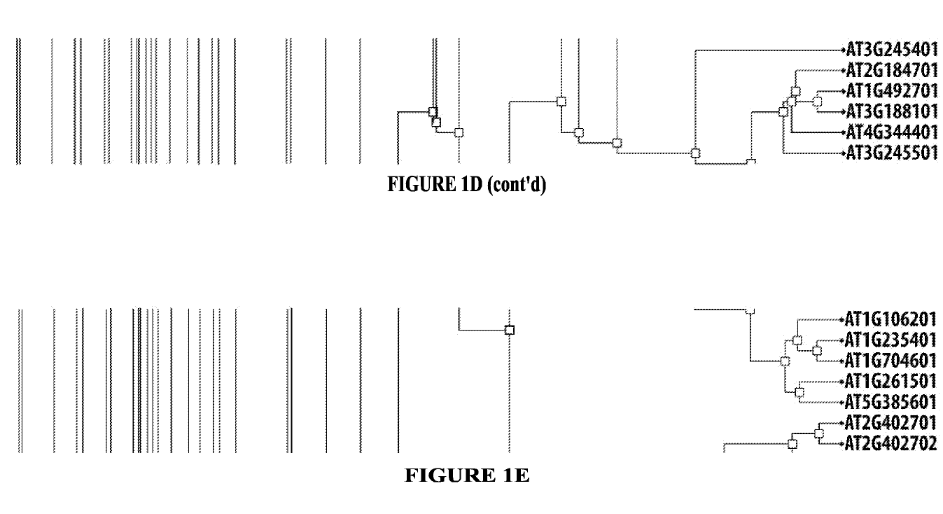

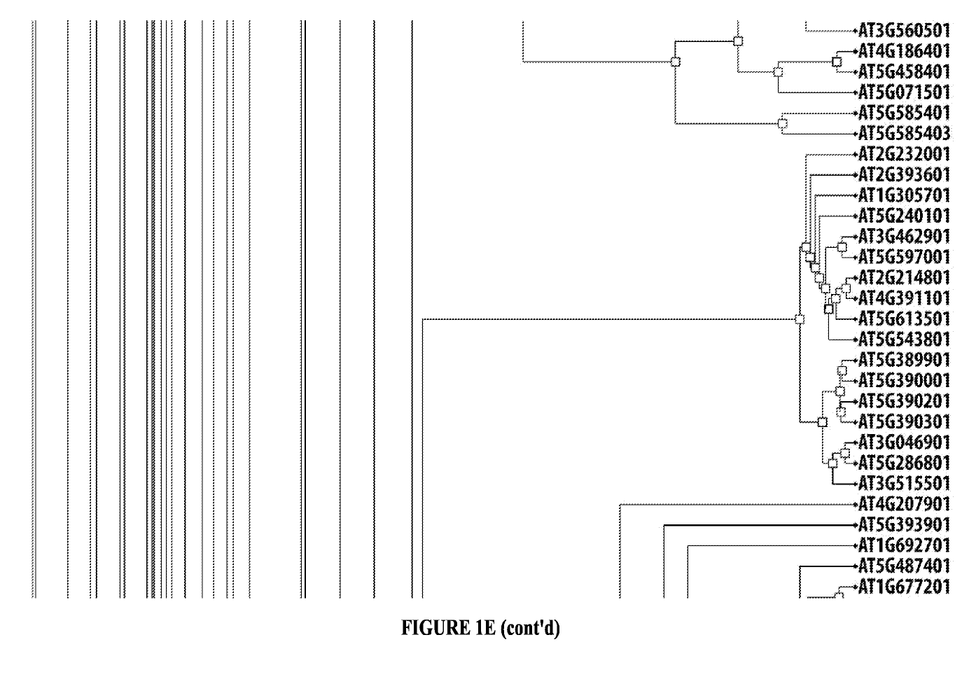

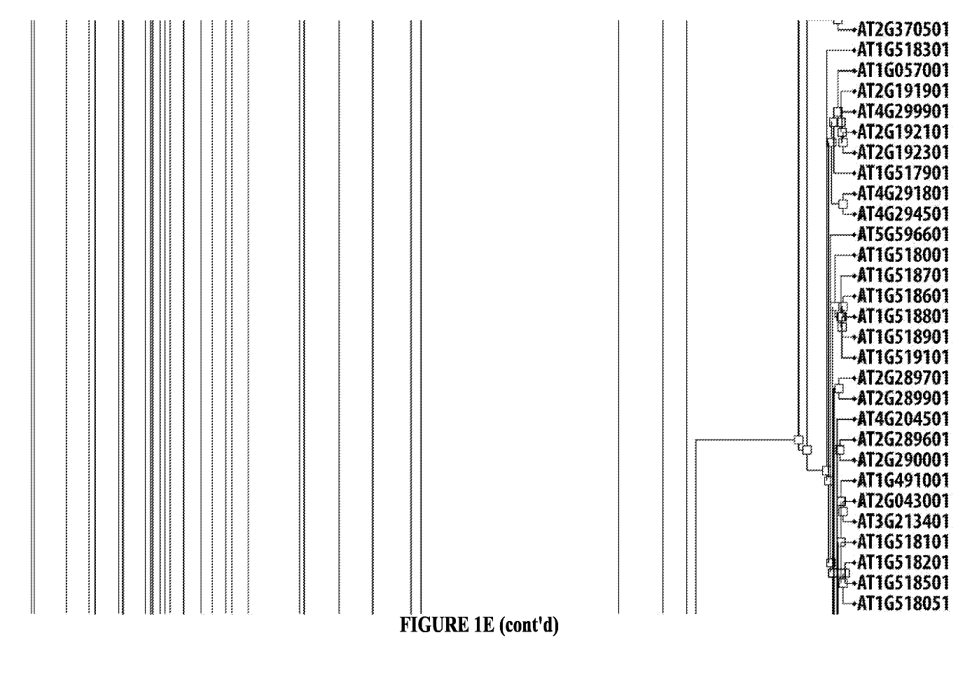

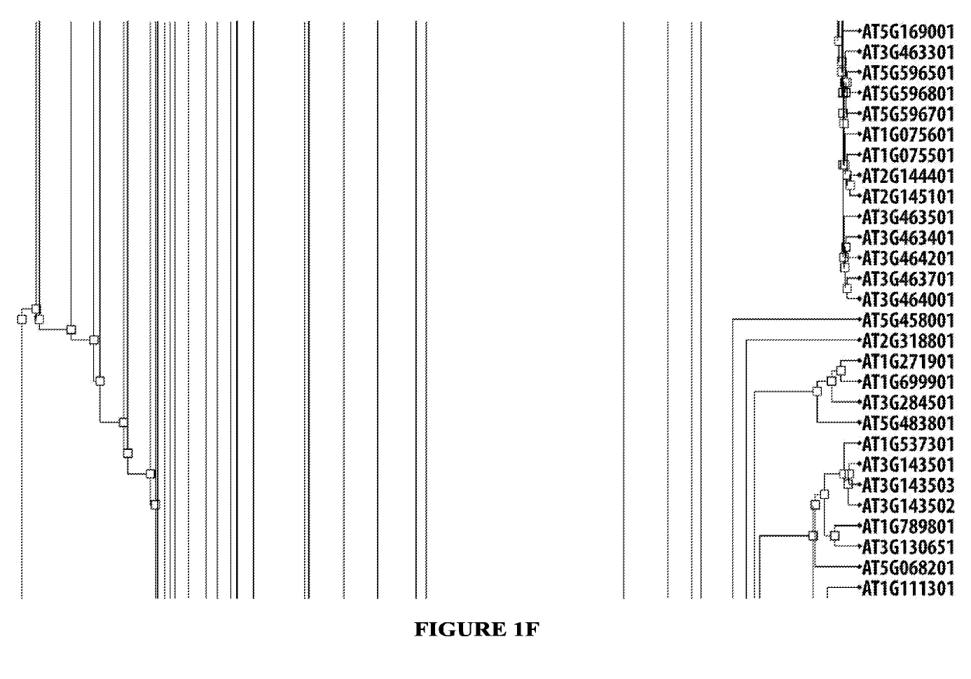

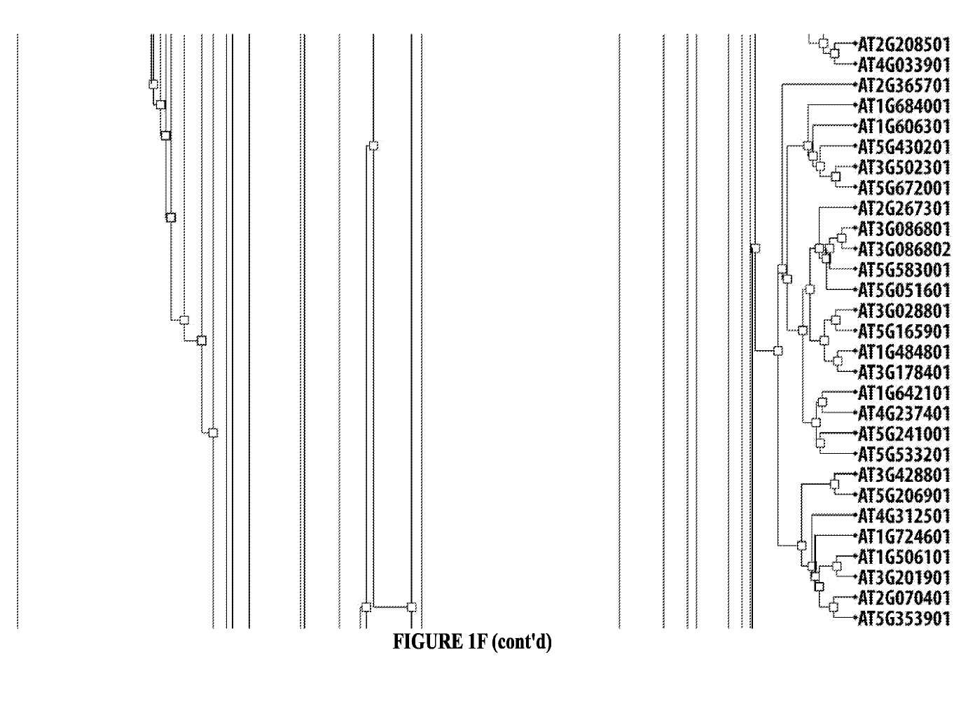

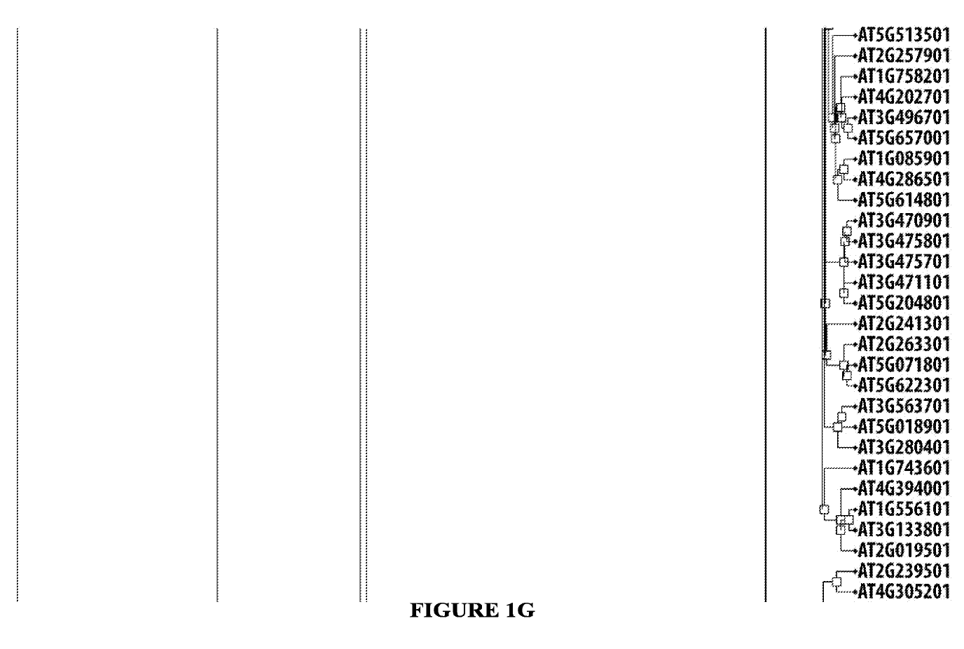

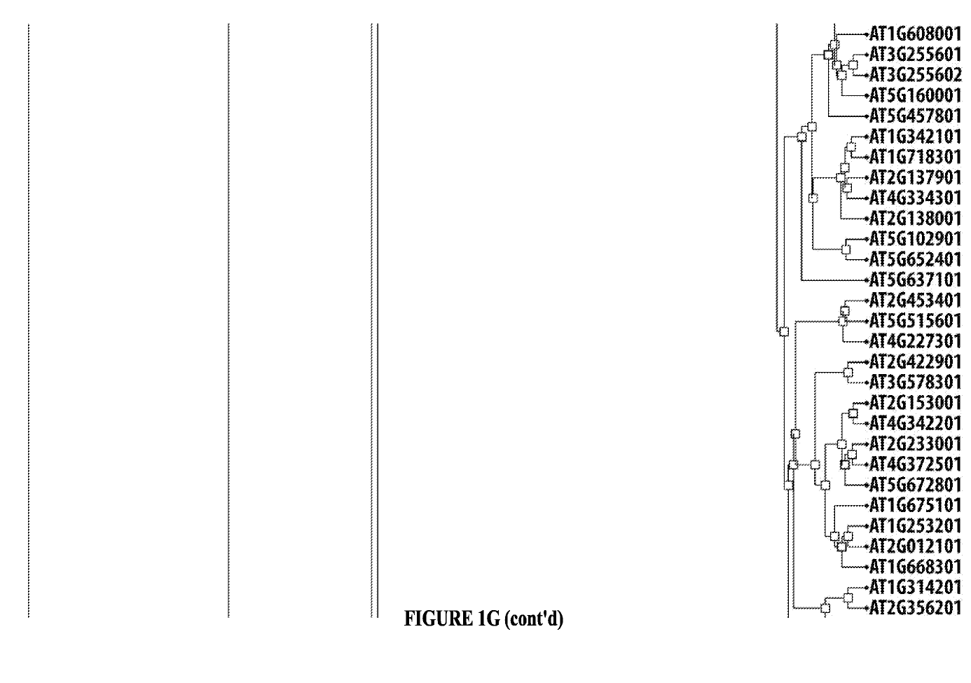

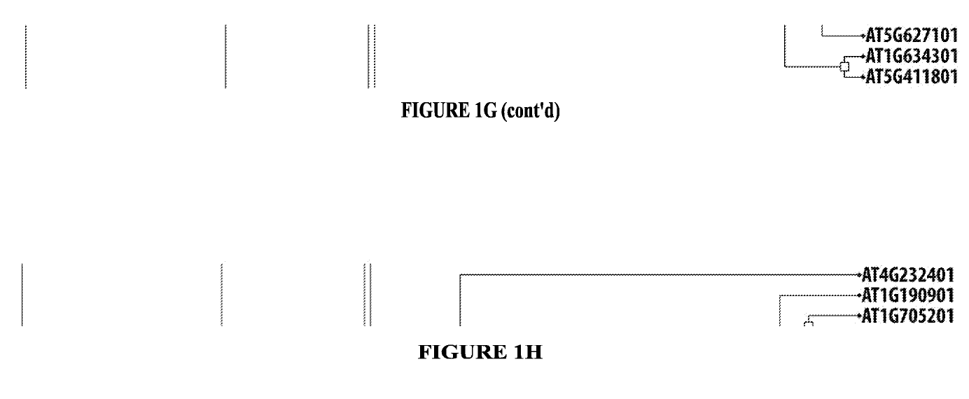

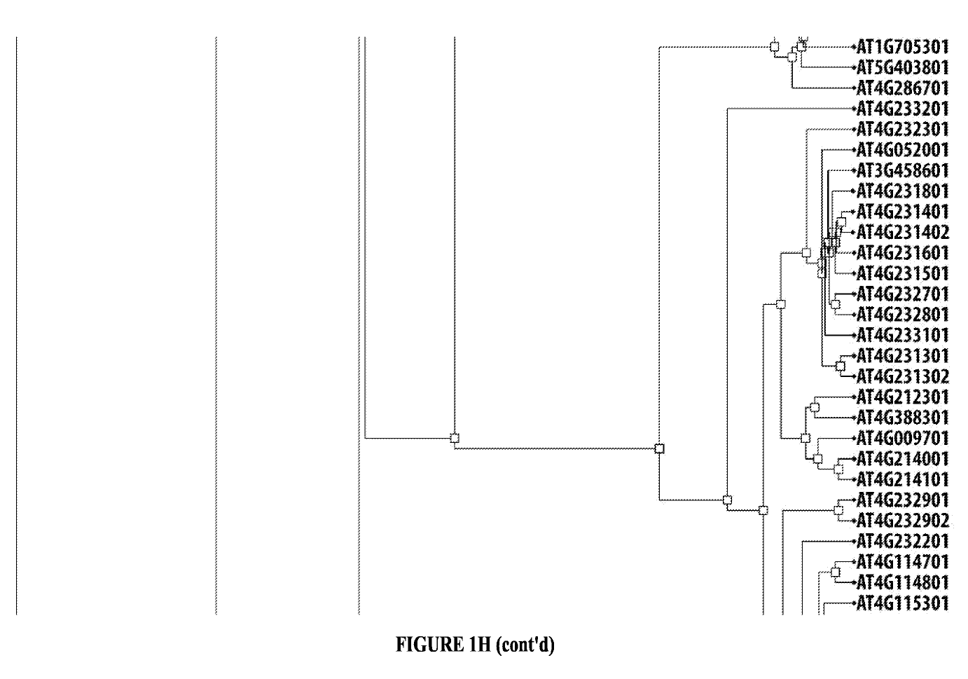

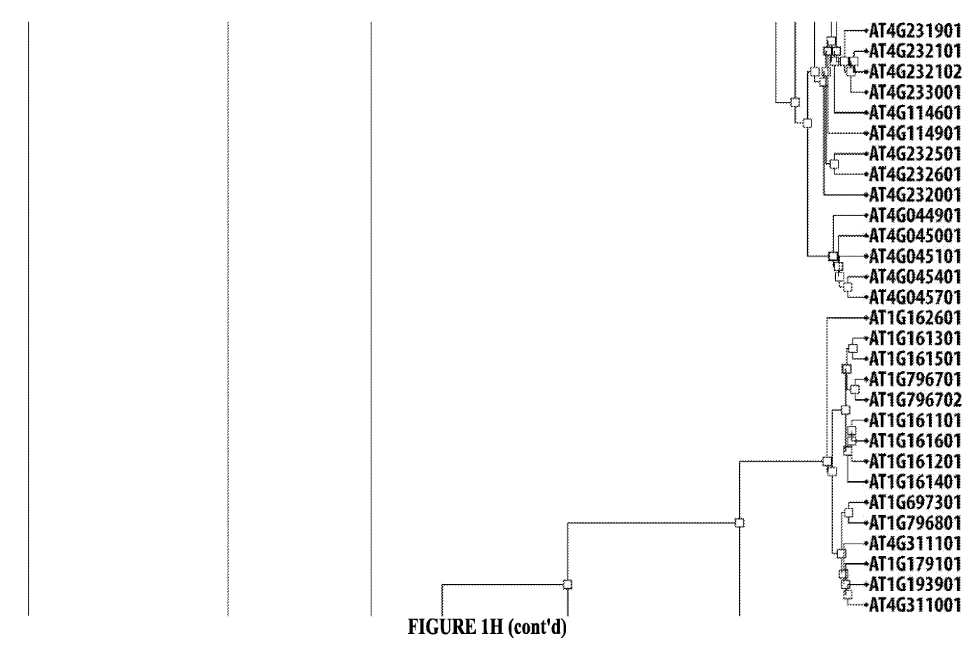

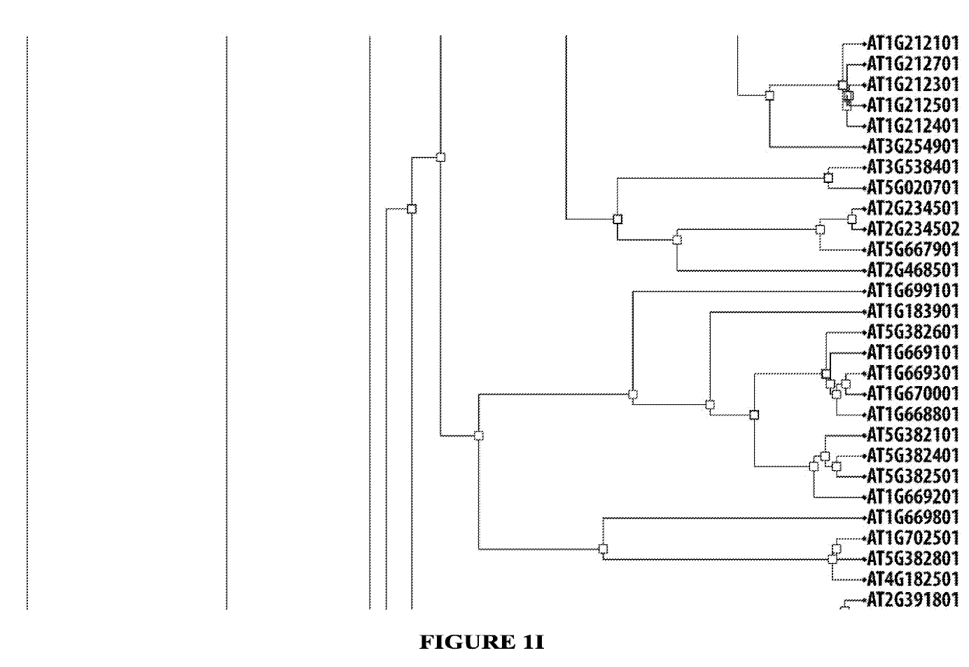

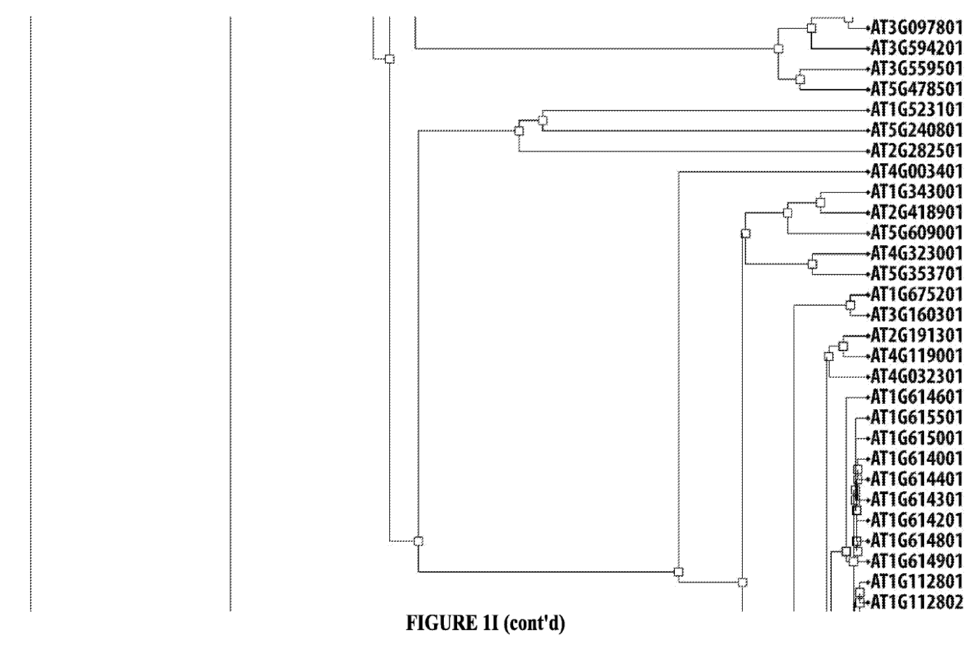

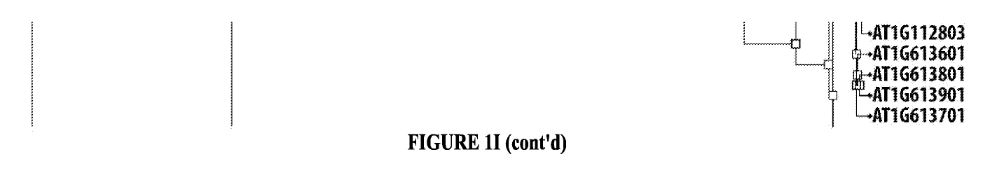

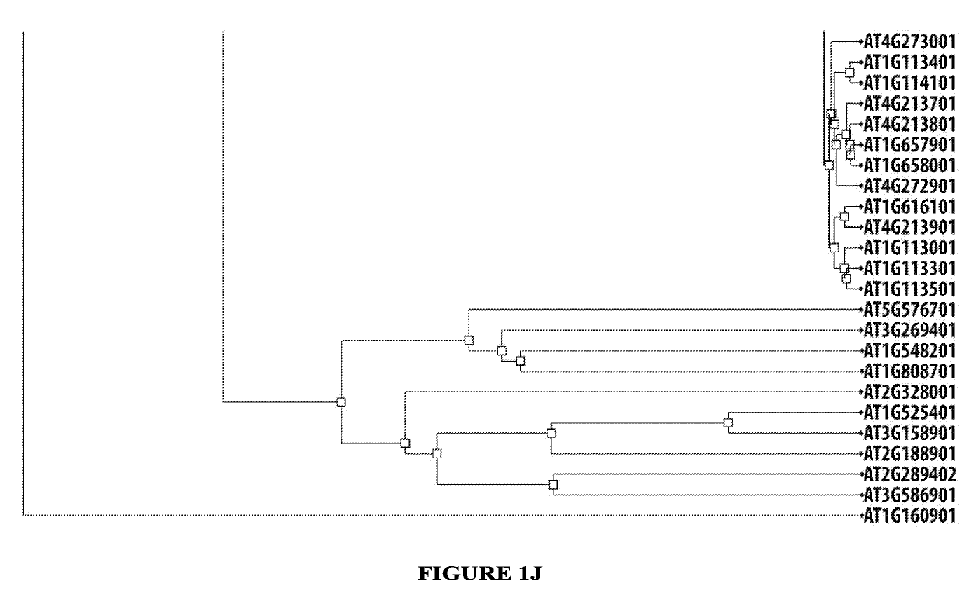

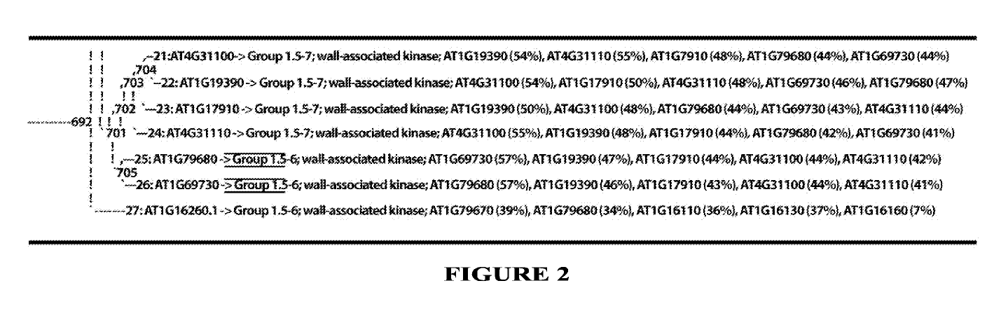

The dominant negative receptor kinases methods and compositions provided by the disclosure allow for the perturbation of the function of many subfamily members at once. The preliminary steps involved compiling all of the known RLKs (˜600) from the publicly available databases (TAIR and PlantsP) and journal articles (Shiu and Bleecker, supra). These were then aligned using the extracellular and transmembrane domains only and a distance map was generated (FIG. 1). This distance map was used to group RLKs into over 250 subfamilies (Table 1). Subfamily categories were determined by a nearest neighbor alignment that looks at the percent shared identity to the adjacent RLKs, all neighbors with over 50% identity were classified as being in the same subfamily (FIG. 2), this alignment is available on the website, (http:˜˜bioinfo.ucr.edu/projects/RLK/Analyses/Final/DecisionTree.html). FIG. 2 is an example of how the nearest neighbor distance map was used to generate the RLK subfamilies. In this example a section of the family was used to demonstrate how the protein similarities in the extracellular domain were used to generate the subfamilies. Subfamily 1.5-7 (Group 1.5-7) contains four genes (At4g31100, At1g19390, At1g17910, At4g31110) that are all greater then 50% identical to each other but less then 50% identical to subfamily 1.5-6 (At1g79680, At1g69730) and 1.5-2 (At1g16260). This method was used on all the RLKs to generate the subfamilies used in this study (FIG. 2).

Upon further investigation RLKs without predicted transmembrane domains (137 RLKs) or of less than 450 amino acids in length (122 RLKs) or in the class of receptor-like cytoplasmic kinases (113 RLCKs), were removed which left 430 RLKs that constituted 157 RLK subfamilies. It was these 157 subfamilies that were used to generate the 72 dominant negative RLK lines.

The disclosure is based in part upon the hypothesis that the overexpression of dominant negative would act as either a ligand trap by binding up free ligands to a catalytically inactive RLK and/or form a dimer with the native RLK but be unable to propagate a signal because there was no active kinase domain to transphosphorylate (FIG. 3). In subfamilies with many members the dominant negative can homo/heterodimerize with other subfamily members and attenuate the signal and thereby allow for determination of the function of that RLK subfamily.

Furthermore, gene expression data (via Genevestigator) was used to better target searches for RLK gene function (FIG. 4). The meta analyzer tool available on the Genevestigator website, https:˜˜www.genevestigator.ethz.ch, to enter in the AGI numbers of all of the RLKs (the maximum allowed at one time is 100) and analyze the expression patterns in each of three categories: developmental stages, tissue regions and biotic and abiotic elicitors (these can be: hormones/chemicals, light, nutrients as well as pathogens). This approach allowed a look at DN-RLK lines that showed no apparent phenotype when grown under normal growth conditions and to use sensitized screening to elucidate phenotypes. This approach also allowed us to look for other RLKs that may have similar functions based on similar expression patterns.

Seventy-two different DN-RLK constructs, which represents 72 subfamilies of RLKs that effectively encompass 45.9% of the RLKs were generated in Arabidposis that fit the initial cutoff criteria (Table 2). Initially the expression levels of the DN-RLKs were examined to determine if the expression levels of the DN-RLKs were detectible and expressed above wild type levels using semi-quantitative RT-PCR. In all cases the DN-RLK transgenic lines had higher then wild type gene expression. For each experiment the maximum number of independent lines used was five unless there were only fewer than those amounts.

Of the 72 DN-RLK subfamilies examined on soil only 23.6% (17 out of 72) showed a developmental or morphological phenotype. When using more selective growing conditions (nutrient deprivation, light regimes or detailed root examination) many more phenotypes were found, with about 64% (37 out of 58, 14 were not examined) showing a phenotype (Table 2). Previously, it was shown that the dominant negative approached worked but this was limited to the family of receptor-like kinases called leucine-rich repeat (LRR) RLKs (Steak et al., 2003). Over half of the DN-RLKs examined (39 of 72) were not LRR-RLKs (Table 2). It appears that the DN approach will also work on non-LRR-RLKs, which makes it an excellent tool for examining RLK function.

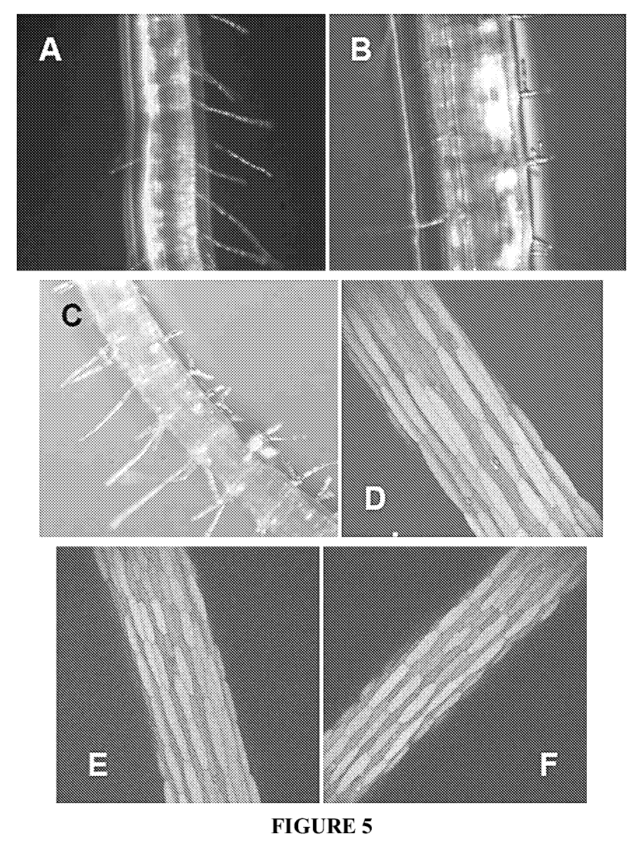

FIG. 5 examines two dominant negative constructs that showed morphological phenotypes that were then confirmed using knockout mutants. The first DN-RLK (1.12-23, At5g01890) was from a LRR-RLK subfamily containing 3 members. All independent lines exhibited a root hair phenotype where the root hairs were shorter and thicker then wild type and were branched (FIG. 5B). A homozygous knockout line was obtained from the ARBC (At3g28040, SALK 093189) and this mutant also had this same root hair phenotype, only not a severe as the DN (FIG. 5C). The difference in severity of phenotype is probably due to the DN having a stronger effect than the single knockout. This again illustrates the utility of the DN approach for overcoming functional redundancy. The other DN-RLK construct is a member of the Strubbelig Receptor Family (SRF) and exhibited a change in hypocotyl epidermal cell size and shape. This gene subfamily only contains two members (At3g14350 and At1g53730). In the wild type the epidermal cells are long and rectangular, however in the DN the epidermal cells are smaller and more square-like (FIGS. 5D/E).

The most common morphological phenotypes observed when grown on soil were changes in leaf shape, size or number as well as a delay in flowering time compared to the wild type. Out of the 72 DN-RLK constructs only two showed a reduction in leaf size (1.3-9, At5g49760; 1.5-5, At1g16110) while fives showed and increase in leaf size (1.1-2, At3g21630; 1.1-4, At3g14350; 1.9-1, At5g38990; 1.9-7, At1g34300; 1.12-30, At5g62710) (Table 2). A delay in flowering time over one week more than the wild type plants was the most common morphological phenotype with 6 different DN-RLK constructs showing a delay in flowering phenotype (1.2-31, At2g28250; 1.7-10, At1g70520; 1.9-1, At5g38990; 1.9-7, At1g34300; 1.9-8, At4g32300; 1.14-5, At1g78940) (Table 2).

When seedlings were grown under limiting conditions (e.g., nutrient deprivation) on Petri dishes the phenotypes of all of the DN-RLKs was very reproducible from one experiment to the next. The most variability of phenotypes from one growing period to the next was when the DN-RLKs were grown on soil. This may be due to the differences in temperature, light quality and watering frequency from one time to the next. In cases where there are many different independent lines (>10) for a DN-RLK construct a gradation in the severity of the phenotype was observed. This may be due to differences in DN-RLK expression levels based on the region of the transgenes insertion into the genome. Otherwise the phenotypes of the DN-RLK constructs are very reproducible and consistent when growth conditions can be rigorously maintained.

TABLE 1
Receptor-like kinases from Arabidopsis arranged by PlantsP family
and subfamily, based on extracellular domain. Names were from TAIR
website. For those with no name currently: PK = protein kinase; LRR =
leucine rich repeat receptor-like kinase. Transmembrane domain (TMD)
prediction was determined using the HMMTOP (http://hmmtop.enzim.hu/) and
the region in parentheses was the amino acid residues predicted to be in
the membrane. Size is the predicted amino acid number for the protein.
AGI# is the TAIR classification. PNAS is the functional classification
found in Shiu and Bleecker (2001). Tree position is the location of the
RLK in the distance map (FIG. 2.1)

| | | | | | | |
|---|---|---|---|---|---|---|
| | | TMD Predicted | Size | | | Tree |
| ID # | Name | (HMMTOP) | (aa) | AGI# | PNAS | Position |

Family 1.Other

| | | | | | | |
|---|---|---|---|---|---|---|
| 1.Other-1 | PK | N | 351 | At4g11890 | DUF26 | 489 |
| 1.Other-2 | PK | N | 377 | At5g60080 | NF | N.A. |
| 1.Other-2 | PK | N | 398 | At5g60090 | NF | N.A. |
| 1.Other-3 | PK | N | 312 | At5g11400 | RLCK II | 593 |
| 1.Other-3 | PK | N | 336 | At5g11410 | RLCK II | 592 |
| 1.Other-4 | PK | Y (9-31; 156-178) | 361 | At5g61570 | LRR III | 330 |
| 1.Other-4 | PK | Y (4-26) | 359 | At5g07620 | LRR III | 331 |
| 1.Other-5 | PK | Y (7-30; 85-108) | 359 | At5g42440 | LRR X | 396 |
| 1.Other-5 | PK | Y (7-29) | 332 | At5g46080 | N.A. | 91 |
| 1.Other-6 | PK | Y (54-78) | 445 | At2g30940.1 | TAKL | 125 |
| 1.Other-6 | PK | Y (54-78) | 447 | At2g30940.2 | TAKL | 125 |
| 1.Other-7 | PK | Y (4-27) | 380 | At3g26700 | RLCK IX | 572 |
| 1.Other-8 | PK | N | 557 | At3g08760 | N.A. | N/A |
| 1.Other-9 | LRR | Y (6-29; 192-210; | 518 | At4g20790 | LRR VI | 587 |
| | | 217-235) | | | | |
| 1.Other- | LRR | Y (6-23; 173-190; | 502 | At5g39390 | LRR XII | 547 |
| 10 | | 203-220) | | | | |
| 1.Other- | LRR | Y (297-320; 370-393) | 666 | At5g45800 | LRR VII | 339 |
| 11 | | | | | | |
| 1.Other- | ERL P | Y (602-621) | 1048 | At5g10020 | LRR III | 334 |
| 12 | | | | | | |
| 1.Other- | LRR | Y (553-570) | 1007 | At2g27060 | LRR III | 335 |
| 12 | | | | | | |
| 1.Other- | InRPK1 | Y (614-638) | 977 | At4g20940 | LRR III | 333 |
| 12 | | | | | | |
| 1.Other- | EPL P | Y (8-31; 246-263; | 633 | At2g46850 | N.A. | 539 |
| 13 | | 284-307) | | | | |
| 1.Other- | Duel PKD | Y (718-737) | 851 | At2g32800 | L-Lectin | 535 |
| 14 | | | | | | |
| 1.Other- | PK | N | 350 | At1g52540 | N.A. | 540 |
| 15 | | | | | | |

Family
1.1

| | | | | | | |
|---|---|---|---|---|---|---|
| 1.1-1 | SRF8 | N | 338 | At4g22130 | LRR V | 94 |
| 1.1-2 | PK | Y (6-23; 234-252; | 617 | At3g21630 | LysM | 285 |
| | | 372-389) | | | | |
| 1.1-2 | RLK | Y (121-145; 237-260) | 657 | At1g51940 | LysM | 286 |
| | (LysM) | | | | | |
| 1.1-3 | PK | Y (243-262; 506-525) | 654 | At3g01840 | N.A. | 603 |
| 1.1-3 | RLK | N | 612 | At2g23770 | LysM | 605 |
| | (LysM) | | | | | |
| 1.1-3 | RLK | Y (121-145; 237-260) | 651 | At2g33580 | LysM | 604 |
| | (LysM) | | | | | |
| 1.1-4 | SRF7 | Y (288-312) | 717 | At3g14350.1 | LRR V | 93 |
| 1.1-4 | SRF7 | Y (251-275) | 680 | At3g14350.2 | LRR V | 93 |
| 1.1-4 | SRF7 | Y (288-312) | 689 | At3g14350.3 | LRR V | 93 |
| 1.1-4 | SRF6 | Y (291-314) | 719 | At1g53730 | LRR V | 92 |
| 1.1-5 | SRF5 | Y (267-291) | 693 | At1g78980 | LRR V | 99 |
| 1.1-5 | SRF4 | Y (233-257) | 646 | At3g13065 | LRR V | 98 |
| 1.1-6 | SRF2 | Y (294-318) | 735 | At5g06820 | LRR V | 100 |
| 1.1-7 | SRF3 | Y (7-29; 36-58; | 776 | At4g03390 | LRR V | 95 |
| | | 317-339) | | | | |
| 1.1-7 | SRF9 | Y (8-26; 342-360; | 768 | At1g11130 | NF | N.A. |
| | (SUB) | 472-490) | | | | |
| 1.1-7 | SRF1 | Y (9-28; 312-331) | 772 | At2g20850 | LRR V | 96 |

Family
1.2

| | | | | | | |
|---|---|---|---|---|---|---|
| 1.2-1 | Pto KI 1 P | N | 406 | At2g43230 | RLCKVIII | 69 |
| 1.2-1 | Pto KI 1 P | N | 408 | At3g59350.1 | RLCKVIII | 70 |
| 1.2-1 | Pto KI 1 P | N | 366 | At3g59350.2 | RLCKVIII | 70 |
| 1.2-2 | Pto KI 1 P | N | 361 | At1g06700 | RLCKVIII | 67 |
| 1.2-2 | Pto KI 1 P | N | 366 | At2g30740 | RLCKVIII | 66 |
| 1.2-3 | Pto KI 1 P | N | 338 | At2g30730 | RLCKVIII | 68 |
| 1.2-4 | Pto KI 1 P | N | 365 | At2g41970 | RLCKVIII | 75 |
| 1.2-5 | Pto KI 1 P | N | 363 | At1g48210 | RLCKVIII | 73 |
| 1.2-5 | Pto KI 1 P | N | 388 | At1g48220 | RLCKVIII | 76 |
| 1.2-5 | Pto KI 1 P | N | 364 | At3g17410 | RLCKVIII | 74 |
| 1.2-6 | Pto KI 1 P | N | 365 | At2g47060.1 | RLCKVIII | 71 |
| 1.2-6 | Pto KI 1 P | N | 397 | At2g47060.2 | RLCKVIII | 71 |
| 1.2-6 | Pto KI 1 P | N | 361 | At3g62220 | RLCKVIII | 72 |
| 1.2-7 | APK1A P | N | 375 | At1g24030 | RLCKVII | 46 |
| 1.2-8 | PK | N | 442 | At2g07180 | RLCKVII | 20 |
| 1.2-8 | PK | Y (268-287) | 450 | At1g72540 | RLCKVII | 24 |
| 1.2-8 | PK | Y (175-197) | 408 | At5g56460 | RLCKVII | 22 |
| 1.2-9 | PK | N | 202 | At1g61590 | RLCKVII | 23 |
| 1.2-10 | PK | N | 462 | At2g05940 | RLCKVII | 18 |
| 1.2-10 | PK | N | 457 | At5g35580 | RLCKVII | 17 |
| 1.2-10 | PK | N | 424 | At2g26290 | RLCKVII | 19 |
| 1.2-11 | PK | Y (274-291) | 410 | At5g47070 | RLCKVII | 30 |
| 1.2-11 | PK | N | 388 | At4g17660 | RLCKVII | 29 |
| 1.2-12 | APK1A P | Y (238-257) | 490 | At3g01300 | RLCKVII | 6 |
| 1.2-12 | APK1A P | N | 493 | At5g15080 | RLCKVII | 7 |
| 1.2-13 | APK1A P | Y (81-100) | 376 | At3g28690 | RLCKVII | 8 |
| 1.2-14 | LMBR1 | Y (18-41; 133-156; | 310 | At3g08930.1 | NF | N.A. |
| | | 177-201; | | | | |
| | | 222-245; 276-297) | | | | |
| 1.2-14 | LMBR1 | Y (6-28; 45-62; | 526 | At3g08930.2 | NF | N.A. |
| | | 89-111; 126-150; | | | | |
| | | 235-257; 349-372; | | | | |
| | | 399-423; | | | | |
| | | 438-461; 492-513) | | | | |
| 1.2-14 | PK | N | 435 | At2g39110 | RLCKVII | 27 |
| 1.2-14 | PK | N | 420 | At5g03320 | RLCKVII | 26 |
| 1.2-15 | PK | N | 399 | At1g74490 | RLCKVII | 13 |
| 1.2-16 | APK2B | N | 426 | At2g02800.1 | RLCKVII | 10 |
| 1.2-16 | APK2B | N | 426 | At2g02800.2 | RLCKVII | 10 |
| 1.2-16 | APK2B | N | 426 | At1g14370 | RLCKVII | 9 |
| 1.2-17 | PK | N | 412 | At1g26970 | RLCKVII | 11 |
| 1.2-17 | APK1A P | N | 387 | At1g69790 | RLCKVII | 12 |
| 1.2-18 | APK1A | N | 410 | At1g07570.1 | RLCKVII | 2 |
| 1.2-18 | APK1A | N | 410 | At1g07570.2 | RLCKVII | 2 |
| 1.2-18 | APK1A/B P | Y (11-27) | 423 | At2g28930 | RLCKVII | 1 |
| 1.2-19 | PK | N | 389 | At5g02290.1 | RLCKVII | 3 |
| 1.2-19 | PK | N | 389 | At5g02290.2 | RLCKVII | 3 |
| 1.2-20 | BIK1 | N | 395 | At2g39660 | RLCKVII | 4 |
| 1.2-20 | APK2B P | N | 389 | At3g55450 | RLCKVII | 5 |
| 1.2-21 | APK1A P | Y (280-297) | 414 | At2g17220.1 | RLCKVII | 14 |
| 1.2-21 | APK1A P | Y (279-296) | 413 | At2g17220.2 | RLCKVII | 14 |
| 1.2-21 | PK | Y (278-294) | 419 | At4g35600 | RLCKVII | 16 |
| 1.2-22 | PK | Y (284-303) | 423 | At1g07870 | RLCKVII | 35 |
| 1.2-22 | PK | N | 424 | At2g28590 | RLCKVII | 34 |
| 1.2-23 | PK | N | 386 | At3g20530 | RLCKVII | 36 |
| 1.2-23 | RLK | N | 389 | At1g61860 | RLCKVII | 40 |
| 1.2-24 | RLK | N | 585 | At1g20650 | RLCKVII | 42 |
| 1.2-24 | APK2B P | N | 381 | At1g76370 | RLCKVII | 41 |
| 1.2-25 | PK | N | 379 | At3g24790 | RLCKVII | 39 |
| 1.2-26 | PBS1 | N | 456 | At5g13160 | RLCKVII | 32 |
| 1.2-26 | PK | N | 378 | At5g02800 | RLCKVII | 33 |
| 1.2-26 | PK | N | 513 | At5g18610 | RLCKVII | 31 |
| 1.2-27 | PK | Y (247-266) | 558 | At3g02810 | RLCKVII | 43 |
| 1.2-27 | PK | Y (260-279) | 414 | At3g07070 | RLCKVII | 37 |
| 1.2-27 | PK | Y (258-275) | 636 | At5g16500 | RLCKVII | 44 |
| 1.2-28 | PK | N | 410 | At5g01020 | RLCKVII | 21 |
| 1.2-28 | TSL | Y (399-416) | 688 | At5g20930 | N.A. | N.A. |
| 1.2-29 | PK | Y (6-30; 259-281) | 744 | At2g20300 | Extensin | 78 |
| 1.2-29 | NF | NF | ?? | At4g02101 | Extensin | 79 |
| 1.2-29 | PK | Y (568-585; 629-652) | 1113 | At5g56890 | Extensin | 77 |
| 1.2-30 | PK | N | 484 | At1g76360 | RLCKVII | 15 |
| 1.2-31 | RERK1 L | Y (71-90; 103-122; | 565 | At2g28250 | N.A. | 82 |
| | | 392-411) | | | | |
| 1.2-32 | CDG1 | N | 432 | At3g26940 | RLCKVII | 45 |
| 1.2-33 | PK | N | 343 | At2g28940 | RLCKVII | 28 |
| 1.2-34 | PBS1 P | N | 405 | At4g13190 | RLCKVII | 38 |

Family
1.3

| | | | | | | |
|---|---|---|---|---|---|---|
| 1.3-1 | PK | Y (7-28) | 261 | At5g54590.1 | LRRI | 225 |
| 1.3-1 | PK | Y (8-30) | 440 | At5g54590.2 | LRRI | 225 |
| 1.3-2 | AtPK2324L | Y (7-26) | 663 | At1g49730.1 | URK1 | 275 |
| 1.3-2 | AtPK2324L | Y (7-26; 256-275; | 450 | At1g49730.2 | URK1 | 275 |
| | | 322-341) | | | | |
| 1.3-2 | AtPK2324L | Y (200-219; 266-285) | 394 | At1g49730.3 | URK1 | 275 |
| 1.3-2 | PK | Y (8-25; 258-275) | 663 | At3g19300 | URK1 | 276 |
| 1.3-3 | CRPK1L-1 | Y (0-27; 339-356; | 824 | At5g24010 | CrRLK1L-1 | 198 |
| | | 408-432) | | | | |
| 1.3-3 | PK | Y (407-426; 472-488) | 834 | At2g23200 | CrRLK1L-1 | 207 |
| 1.3-3 | CRPK1L-1 | Y (408-432; 463-480) | 815 | At2g39360 | CrRLK1L-1 | 206 |
| 1.3-3 | PK | Y (8-24; 386-402; | 849 | At1g30570 | CrRLK1L-1 | 202 |
| | | 431-455) | | | | |
| 1.3-4 | CRPK1L-1 | Y (6-23) | 829 | At5g59700 | CrRLK1L-1 | 196 |
| 1.3-4 | PK | Y (8-25; 404-428; | 830 | At3g46290 | CrRLK1L-1 | 195 |
| | | 441-465) | | | | |
| 1.3-5 | PK | Y (21-43; 439-461; | 871 | At2g21480 | CrRLK1L-1 | 199 |
| | | 476-493) | | | | |
| 1.3-5 | PK | Y (23-45; 440-462; | 878 | At4g39110 | CrRLK1L-1 | 200 |
| | | 477-494) | | | | |
| 1.3-6 | CRPK1L-1 | Y (424-446; 499-516) | 842 | At5g61350 | CrRLK1L-1 | 201 |
| 1.3-6 | PK (THE1) | Y (7-26; 314-338; | 855 | At5g54380 | CrRLK1L-1 | 197 |
| | | 418-442) | | | | |
| 1.3-7 | FERONIA | Y (11-28; 447-470; | 895 | At3g51550 | CrRLK1L-1 | 205 |
| | | 485-502) | | | | |
| 1.3-7 | PK | N | 850 | At3g04690 | CrRLK1L-1 | 203 |
| 1.3-7 | PK | Y (7-23) | 858 | At5g28680 | CrRLK1L-1 | 204 |
| 1.3-8 | LRR | Y (55-77; 88-104; | 1032 | At5g01950 | LRR | 211 |
| | | 643-665) | | | VIII-1 | |
| 1.3-8 | LRR | Y (546-570) | 939 | At1g06840 | LRR | 212 |
| | | | | | VIII-1 | |
| 1.3-8 | LRR | Y (537-561) | 935 | At5g37450 | LRR | 213 |
| | | | | | VIII-1 | |
| 1.3-8 | LRR CLV1 P | Y (376-394) | 783 | At3g53590 | LRR | 210 |
| | | | | | VIII-1 | |
| 1.3-9 | RLK (LRR- | Y (8-25; 514-537; | 953 | At5g49760 | LRR | 214 |
| | VIII-1) | 558-582) | | | VIII-1 | |
| 1.3-9 | RLK (LRR- | Y (7-26; 562-585; | 946 | At5g49770 | LRR | 215 |
| | VIII-1) | 616-634) | | | VIII-1 | |
| 1.3-9 | LRR | Y (612-633; 683-702) | 1006 | At5g49780 | LRR | 216 |
| | | | | | VIII-1 | |
| 1.3-9 | LRR | ND | ND | At1g79620.1 | LRR | 217 |
| | | | | | VIII-1 | |

Family
1.4

| | | | | | | |
|---|---|---|---|---|---|---|
| 1.4-1 | PK | Y (7-31 395-411 | 776 | At2g39180 | CR4L | 86 |
| | | 432-448) | | | | |
| 1.4-1 | PK | Y (24-43 83-100) | 775 | At3g09780 | CR4L | 87 |
| 1.4-1 | ACR4 | Y (17-39 437-455) | 895 | At3g59420 | CR4L | 88 |
| 1.4-2 | PK | Y (6-28) | 751 | At5g47850 | CR4L | 89 |
| 1.4-2 | NF | NF | ND | At2g55950 | CR4L | 90 |

Family
1.5

| | | | | | | |
|---|---|---|---|---|---|---|
| 1.5-1 | CRCK3 | N | 510 | At2g11520 | RLCK IV | 220 |
| 1.5-2 | WAKL8 | Y (316-337; 446-463) | 720 | At1g16260 | WAKL | 178 |
| 1.5-3 | WAKL2 | Y (346-363; 472-489) | 748 | At1g16130 | WAKL | 169 |
| 1.5-3 | WAKL4 | Y (368-392; 498-515) | 779 | At1g16150 | WAKL | 170 |
| 1.5-4 | WAKL22 | Y (6-23; 351-368; | 751 | At1g79670.1 | WAKL | 171 |
| | | 476-493) | | | | |
| 1.5-4 | WAKL22 | Y (6-25; 314-331; | 714 | At1g79670.2 | WAKL | 171 |
| | | 439-456) | | | | |
| 1.5-5 | WAKL6 | Y (362-379; 488-505; | 642 | At1g16110 | WAKL | 168 |
| | | 536-553; | | | | |
| | | 584-601) | | | | |
| 1.5-5 | WAKL5 | Y (340-361; 467-484; | 711 | At1g16160 | WAKL | 167 |
| | | 515-534) | | | | |
| 1.5-5 | WAKL1 | Y (359-376; 485-502; | 730 | At1g16120 | WAKL | 165 |
| | | 533-552) | | | | |
| 1.5-5 | WAKL3 | Y (322-338; 444-460; | 690 | At1g16140 | WAKL | 166 |
| | | 491-510) | | | | |
| 1.5-6 | WAKL9 | Y (373-397; 503-520) | 792 | At1g69730 | WAKL | 176 |
| 1.5-6 | WAKL10 | Y (8-25; 359-383; | 769 | At1g79680 | WAKL | 177 |
| | | 489-506) | | | | |
| 1.5-7 | WAKL17 | Y (369-390; 500-517) | 786 | At4g31100 | WAKL | 172 |
| 1.5-7 | WAKL18 | Y (9-26; 345-362; | 756 | At4g31110 | WAKL | 173 |
| | | 472-489) | | | | |
| 1.5-7 | WAKL13 | Y (7-26; 381-400; | 764 | At1g17910 | WAKL | 175 |
| | | 510-527) | | | | |
| 1.5-7 | WAKL11 | Y (378-397; 507-524) | 788 | At1g19390 | WAKL | 174 |
| 1.5-8 | WAK1 | Y (362-379; 488-505; | 642 | At1g21250 | WAK | 184 |
| | | 536-553; | | | | |
| | | 584-601) | | | | |
| 1.5-8 | WAK4 | N | 738 | At1g21210 | WAK | 180 |
| 1.5-8 | WAK2 | Y (332-350; 371-389) | 732 | At1g21270 | WAK | 181 |
| 1.5-8 | WAK5 | N | 733 | At1g21230 | WAK | 179 |
| 1.5-8 | WAK3 | Y (343-361; 382-400) | 741 | At1g21240 | WAK | 183 |
| 1.5-9 | WAKL16 | Y (6-24; 29-47; | 433 | At3g25490 | WAKL | 185 |
| | | 76-93) | | | | |
| 1.5-10 | WAKL20 | Y (7-24; 293-316; | 657 | At5g02070 | WAKL | 186 |
| | | 418-435) | | | | |
| 1.5-10 | WAKL15 | N | 639 | At3g53840 | WAKL | 187 |
| 1.5-11 | WAKL14 | Y (24-46; 283-306) | 708 | At2g23450.1 | WAKL | 192 |
| 1.5-11 | WAKL14 | Y (24-46; 283-306) | 708 | At2g23450.2 | WAKL | 192 |
| 1.5-11 | WAKL21 | Y (8-26; 248-272; | 622 | At5g66790 | WAKL | 193 |
| | | 283-299) | | | | |
| 1.5-12 | PK | Y (256-275) | 636 | At1g69910 | LRK10L-1 | 194 |
| 1.5-13 | PK | N | 605 | At1g18390 | LRK10L-1 | 189 |
| 1.5-14 | PK | Y (14-31) | 686 | At5g38210 | LRK10L-1 | 190 |

Family
1.6

| | | | | | | |
|---|---|---|---|---|---|---|
| 1.6-1 | PK | Y (8-26; 35-54) | 452 | At5g20050 | N.A. | 148 |
| 1.6-1 | PK | Y (268-287) | 450 | At1g72540 | RLCKVII | 24 |
| 1.6-2 | PK | Y (32-54) | 676 | At1g55200 | PERKL | 63 |
| 1.6-2 | PK | Y (35-57) | 753 | At3g13690 | PERKL | 64 |
| 1.6-2 | PK | Y (110-127; 393-410) | 669 | At5g56790 | PERKL | 65 |
| 1.6-3 | PK | Y (21-45) | 437 | At4g34500 | TAKL | 124 |
| 1.6-4 | PK | Y (26-50) | 512 | At3g59110 | TAKL | 116 |
| 1.6-4 | PK | Y (25-48; 345-362) | 494 | At2g42960 | TAKL | 115 |
| 1.6-5 | GPK1 | Y (21-40) | 467 | At3g17420 | TAKL | 119 |
| 1.6-5 | PK | Y (21-40) | 484 | At5g18500 | TAKL | 120 |
| 1.6-6 | PK | Y (24-48; 210-227) | 386 | At1g01540.1 | TAKL | 122 |
| 1.6-6 | PK | Y (24-48) | 472 | At1g01540.2 | TAKL | 122 |
| 1.6-6 | PK | Y (26-49; 218-235) | 329 | At4g01330 | TAKL | 121 |
| 1.6-6 | PK | Y (22-46) | 492 | At4g02630 | TAKL | 123 |
| 1.6-7 | PK | Y (179-196; 227-250) | 625 | At1g11050 | RKF3L | 163 |
| 1.6-7 | RKF3 | Y (7-24; 169-186; | 617 | At2g48010 | RKF3L | 164 |
| | | 213-231) | | | | |
| 1.6-8 | PK | Y (58-82; 199-216) | 509 | At1g52290 | PERKL | 50 |
| 1.6-9 | PERK3 | Y (124-144) | 509 | At3g24540 | PERKL | 47 |
| 1.6-10 | PERK4 | Y (151-170) | 633 | At2g18470 | PERKL | 54 |
| 1.6-11 | PERK5 | Y (187-209) | 670 | At4g34440 | PERKL | 53 |
| 1.6-11 | PERK7 | Y (175-198) | 699 | At1g49270 | PERKL | 51 |
| 1.6-11 | PERK6 | Y (186-210) | 700 | At3g18810 | PERKL | 52 |
| 1.6-11 | PERK1 | Y (140-162; 336-353) | 652 | At3g24550 | PERKL | 48 |
| 1.6-12 | PERK12 | Y (247-266) | 720 | At1g23540 | PERKL | 58 |
| 1.6-12 | PERK11 | Y (263-282) | 718 | At1g10620 | PERKL | 59 |
| 1.6-12 | PERK13 | Y (236-255) | 710 | At1g70460 | PERKL | 57 |
| 1.6-13 | PERK10 | Y (329-352) | 760 | At1g26150 | PERKL | 60 |
| 1.6-13 | PERK8 | Y (237-259) | 681 | At5g38560 | PERKL | 62 |
| 1.6-14 | TMK1 | Y (6-23; 481-505; | 942 | At1g66150 | LRR IX | 281 |
| | | 539-556) | | | | |
| 1.6-14 | LRR | N | 886 | At1g24650 | LRR IX | 283 |
| 1.6-14 | TMK1L | Y (483-500; 643-660) | 943 | At2g01820 | LRR IX | 282 |
| 1.6-14 | LRR | Y (475-494; 517-534) | 928 | At3g23750 | LRR IX | 284 |

Family
1.7

| | | | | | | |
|---|---|---|---|---|---|---|
| 1.7-1 | LRR | Y (7-24; 89-106) | 112 | At3g14840 | LRR | 470 |
| | | | | | VIII-2 | |
| 1.7-2 | PK | N | 372 | At4g00960 | DUF26 | 424 |
| 1.7-3 | PK | N | 390 | At1g16670 | LRR | 476 |
| | | | | | VIII-2 | |
| 1.7-3 | PK | N | 393 | At3g09010 | LRR | 475 |
| | | | | | VIII-2 | |
| 1.7-4 | PK | Y (235-254) | 425 | At1g70740 | DUF26 | 481 |
| 1.7-5 | LRR | Y (16-35; 562-581; | 1049 | At1g29740 | LRR | 471 |
| | | 600-619) | | | VIII-2 | |
| 1.7-5 | LRR | Y (13-35) | 940 | At1g29730 | LRR | 472 |
| | (RKF1) | | | | VIII-2 | |
| 1.7-6 | LRR | Y (624-643; 723-742) | 1014 | At1g07650 | LRR | 468 |
| | | | | | VIII-2 | |
| 1.7-6 | LRR | Y (571-590; 603-622; | 1030 | At1g53430 | LRR | 467 |
| | | 840-859) | | | VIII-2 | |
| 1.7-6 | LRR | Y (10-29; 576-595; | 1035 | At1g53440 | LRR | 466 |
| | | 608-627) | | | VIII-2 | |
| 1.7-7 | LRR | Y (7-24; 89-106) | 112 | At3g14840 | LRR | 470 |
| | | | | | VIII-2 | |
| 1.7-7 | LRR | Y (6-23; 569-586; | 953 | At1g53420 | LRR | 469 |
| | | 607-624) | | | VIII-2 | |
| 1.7-8 | LRR | N | 1032 | At1g56140 | LRR | 480 |
| | | | | | VIII-2 | |
| 1.7-8 | LRR | Y (7-24; 605-624; | 1032 | At1g56130 | LRR | 478 |
| | | 637-656) | | | VIII-2 | |
| 1.7-8 | LRR | Y (618-637; 650-673) | 1045 | At1g56120 | LRR | 479 |
| | | | | | VIII-2 | |
| 1.7-9 | CRK16 | N | 352 | At4g23240 | DUF26 | 419 |
| 1.7-10 | CRK2 | Y (260-284; 327-344) | 649 | At1g70520 | DUF26 | 485 |
| 1.7-10 | CRK1 | Y (6-23) | 600 | At1g19090 | DUF26 | 484 |
| 1.7-10 | CRK3 | Y (259-283; 296-312) | 646 | At1g70530 | DUF26 | 483 |
| 1.7-10 | CRK42 | Y (192-216; 260-282) | 591 | At5g40380 | DUF26 | 482 |
| 1.7-10 | PK | Y (256-273; 387-404) | 625 | At4g28670 | DUF26 | 486 |
| 1.7-11 | CRK24 | Y (96-115; 132-149) | 416 | At4g23320 | DUF26 | 421 |
| 1.7-12 | CRK10/RLK4 P | Y (11-28) | 669 | At4g23180 | DUF26 | 406 |
| 1.7-12 | CRK25 | Y (8-25; 252-270; | 675 | At4g05200 | DUF26 | 407 |
| | | 283-300) | | | | |
| 1.7-12 | CRK4 | Y (289-306; 361-378) | 676 | At3g45860 | DUF26 | 411 |
| 1.7-13 | CRK6/RLK5 | Y (7-24; 211-228; | 674 | At4g23140.1 | DUF26 | 403 |
| | | 289-306) | | | | |
| 1.7-13 | CRK6/RLK5 | Y (7-24; 211-228; | 680 | At4g23140.2 | DUF26 | 403 |
| | | 289-306) | | | | |
| 1.7-14 | CRK7 | Y (248-265; 274-291) | 659 | At4g23150 | DUF26 | 405 |
| 1.7-14 | CRK8 | Y (577-593; 600-616; | 1262 | At4g23160 | DUF26 | 404 |
| | | 854-870; | | | | |
| | | 877-894) | | | | |
| 1.7-15 | CRK19 | Y (7-26; 263-285; | 645 | At4g23270 | DUF26 | 412 |
| | | 308-327) | | | | |
| 1.7-15 | CRK20 | Y (6-23; 254-277; | 656 | At4g23280 | DUF26 | 408 |
| | | 324-341) | | | | |
| 1.7-16 | RLK4, 5, 6L | Y (6-23; 431-453; | 830 | At4g23310 | DUF26 | 409 |
| | | 488-505) | | | | |
| 1.7-17 | CRK5/RLK6 | Y (252-271; 280-299) | 659 | At4g23130.1 | DUF26 | 410 |
| 1.7-17 | CRK5/RLK6 | Y (252-271; 280-299) | 663 | At4g23130.2 | DUF26 | 410 |
| 1.7-18 | CRK29 | Y (6-23; 287-309; | 679 | At4g21410 | DUF26 | 426 |
| | | 402-419) | | | | |
| 1.7-18 | CRK41 | Y (13-30) | 665 | At4g00970 | DUF26 | 423 |
| 1.7-18 | CRK28 | Y (7-24, 289-311; | 711 | At4g21400 | DUF26 | 425 |
| | | 330-347) | | | | |
| 1.7-19 | CRK21 | Y (192-209; 329-346) | 600 | At4g23290.1 | DUF26 | 420 |
| 1.7-19 | CRK21 | Y (12-29; 282-299; | 690 | At4g23290.2 | DUF26 | 420 |
| | | 419-436) | | | | |
| 1.7-20 | CRK14 | Y (131-148; 159-183) | 542 | At4g23220 | DUF26 | 399 |
| 1.7-21 | CRK32 | Y (6-23; 262-279; | 656 | At4g11480 | DUF26 | 415 |
| | | 366-383) | | | | |
| 1.7-21 | CRK31 | Y (6-23; 221-238; | 666 | At4g11470 | DUF26 | 414 |
| | | 278-301) | | | | |
| 1.7-22 | CRK34 | Y (6-23; 548-569; | 931 | At4g11530 | DUF26 | 400 |
| | | 663-680) | | | | |
| 1.7-23 | CRK33 | Y (7-24; 242-259; | 636 | At4g11490 | DUF26 | 413 |
| | | 266-290) | | | | |
| 1.7-23 | CRK22 | Y (6-24; 291-315; | 660 | At4g23300 | DUF26 | 402 |
| | | 409-426) | | | | |
| 1.7-23 | CRK30 | Y (6-24; 286-304; | 700 | At4g11460 | DUF26 | 416 |
| | | 326-343) | | | | |
| 1.7-24 | CRK17 | Y (8-26; 289-308; | 998 | At4g23250 | DUF26 | 418 |
| | | 385-402; | | | | |
| | | 941-962) | | | | |
| 1.7-24 | CRK18 | Y (208-227; 304-323) | 579 | At4g23260 | DUF26 | 417 |
| 1.7-24 | CRK12 | Y (6-25) | 648 | At4g23200 | DUF26 | 398 |
| 1.7-25 | CRK40 | Y (6-25; 289-308; | 654 | At4g04570 | DUF26 | 430 |
| | | 329-345) | | | | |
| 1.7-25 | CRK36 | Y (6-24; 282-302; | 658 | At4g04490 | DUF26 | 428 |
| | | 325-342) | | | | |
| 1.7-25 | CRK37 | Y (6-24; 288-307; | 646 | At4g04500 | DUF26 | 429 |
| | | 338-357) | | | | |
| 1.7-25 | CRK38 | Y (6-23; 238-255; | 648 | At4g04510 | DUF26 | 432 |
| | | 280-299) | | | | |
| 1.7-25 | CRK39 | Y (6-22; 291-310; | 659 | At4g04540 | DUF26 | 431 |
| | | 333-350) | | | | |
| 1.7-26 | LPK | Y (424-441; 512-528) | 850 | At3g16030 | SD-1 | 449 |
| 1.7-26 | LPK | N | 587 | At1g67520 | SD-1 | 448 |
| 1.7-27 | S-Locus | Y (447-464; 588-605) | 852 | At4g03230 | SD-1 | 435 |
| | LPK | | | | | |
| 1.7-27 | S-Locus | Y (8-25; 468-486; | 849 | At4g11900 | SD-1 | 464 |
| | LPK | 699-716) | | | | |
| 1.7-28 | S-Locus | Y (18-42; 395-412; | 830 | At1g11280.1 | SD-1 | 460 |
| | LPK | 445-462) | | | | |
| 1.7-28 | S-Locus | Y (8-32; 385-402; | 820 | At1g11280.2 | SD-1 | 460 |
| | LPK | 435-452) | | | | |
| 1.7-28 | S-Locus | Y (8-32; 385-402; | 808 | At1g11280.3 | SD-1 | 460 |
| | LPK | 435-452) | | | | |
| 1.7-28 | S-Locus | Y (186-205; 241-260) | 598 | At1g61460 | SD-1 | 463 |
| | LPK | | | | | |
| 1.7-28 | S-Locus | Y (6-29; 367-386; | 802 | At1g61550 | SD-1 | 454 |
| | LPK | 421-440) | | | | |
| 1.7-28 | S-Locus | Y (7-26; 377-394; | 804 | At1g61500 | SD-1 | 451 |
| | LPK | 427-446) | | | | |
| 1.7-28 | S-Locus | Y (20-37; 386-403; | 821 | At1g61400 | SD-1 | 457 |
| | LPK | 436-453) | | | | |
| 1.7-28 | S-Locus | Y (369-386; 419-436) | 792 | At1g61440 | SD-1 | 458 |
| | LPK | | | | | |
| 1.7-28 | S-Locus | Y (7-26; 375-392; | 806 | At1g61430 | SD-1 | 456 |
| | LPK | 425-444) | | | | |
| 1.7-28 | S-Locus | Y (7-26; 371-390; | 807 | At1g61420 | SD-1 | 452 |
| | LPK | 426-445) | | | | |
| 1.7-28 | S-Locus | Y (7-26; 371-390; | 809 | At1g61480 | SD-1 | 453 |
| | LPK | 426-445) | | | | |
| 1.7-28 | S-Locus | Y (7-26; 57-76; | 804 | At1g61490 | SD-1 | 450 |
| | LPK | 83-100; 378-397; | | | | |
| | | 426-445) | | | | |
| 1.7-29 | S-Locus | Y (6-27; 379-400; | 805 | At1g61380 | SD-1 | 461 |
| | LPK | 429-450) | | | | |
| 1.7-29 | S-Locus | Y (7-31; 378-395; | 821 | At1g61360 | SD-1 | 462 |
| | LPK | 428-446) | | | | |
| 1.7-29 | S-Locus | Y (22-39; 396-415; | 831 | At1g61390 | SD-1 | 455 |
| | LPK | 450-468) | | | | |
| 1.7-29 | S-Locus | Y (6-27; 380-399; | 814 | At1g61370 | SD-1 | 459 |
| | LPK | 434-453) | | | | |
| 1.7-30 | S-Locus | Y (6-23; 439-461; | 815 | At4g27300 | SD-1 | 433 |
| | LPK | 492-509) | | | | |
| 1.7-31 | S-Locus | Y (6-26) | 840 | At1g11410 | SD-1 | 437 |
| | LPK | | | | | |
| 1.7-31 | S-Locus | Y (69-86; 99-116) | 901 | At1g11340 | SD-1 | 436 |
| | LPK | | | | | |
| 1.7-32 | S-Locus | Y (10-27) | 850 | At4g21380 | SD-1 | 440 |
| | LPK | | | | | |
| | (ARK3) | | | | | |
| 1.7-32 | S-Locus | Y (11-30; 444-463; | 844 | At4g21370 | SD-1 | 441 |
| | LPK (SRKaP) | 486-502) | | | | |
| 1.7-32 | S-Locus | Y (10-26) | 843 | At1g65790 | SD-1 | 439 |
| | LPK | | | | | |
| | (ARK1) | | | | | |
| 1.7-32 | S-Locus | Y (11-28; 394-411; | 847 | At1g65800 | SD-1 | 438 |
| | LPK | 440-457) | | | | |
| | (ARK2) | | | | | |
| 1.7-33 | S-Locus | Y (9-29) | 772 | At4g27290 | SD-1 | 434 |
| | LPK | | | | | |
| 1.7-34 | S-Locus | Y (446-464; 687-704) | 842 | At1g61610 | SD-1 | 447 |
| | LPK | | | | | |
| 1.7-34 | S-Locus | Y (7-26; 393-410; | 849 | At4g21390 | SD-1 | 446 |
| | LPK | 439-458) | | | | |
| 1.7-35 | S-Locus | Y (435-457; 497-514) | 830 | At1g11350 | SD-1 | 445 |
| | LPK | | | | | |
| 1.7-35 | S-Locus | Y (6-23; 424-441; | 1635 | At1g11300 | SD-1 | 442 |
| | LPK | 479-496; | | | | |
| | | 1252-1269; 1309-1326) | | | | |
| 1.7-34 | S-Locus | Y (445-466; 684-701) | 840 | At1g11330 | SD-1 | 444 |
| | LPK | | | | | |

Family
1.8

| | | | | | | |
|---|---|---|---|---|---|---|
| 1.8-1 | LRR | Y (545-569) | 895 | At5g48740 | LRRI | 223 |
| 1.8-2 | LRR | Y (531-554) | 934 | At2g37050 | LRRI | 221 |
| 1.8-2 | LRR | Y (533-557) | 929 | At1g67720 | LRRI | 222 |
| 1.8-3 | RLK | Y (316-340; 373-390) | 675 | At1g51830 | LRRI | 247 |
| 1.8-4 | RLK | Y (6-25; 514-532; | 843 | At1g05700 | LRRI | 266 |
| | | 549-567) | | | | |
| 1.8-4 | SIRK P | Y (6-22; 519-538; | 876 | At2g19190 | LRRI | 265 |
| | (light- | 569-585) | | | | |
| | responsive) | | | | | |
| 1.8-4 | LRR | Y (516-533; 564-581) | 876 | At4g29990 | LRRI | 264 |
| | (light | | | | | |
| | repressible) | | | | | |
| 1.8-4 | LRR | Y (6-23) | 881 | At2g19210 | LRRI | 262 |
| | (light | | | | | |
| | repressible) | | | | | |
| 1.8-4 | LRR | Y (6-24) | 877 | At2g19230 | LRRI | 263 |
| | (light | | | | | |
| | repressible) | | | | | |
| 1.8-4 | LRR | Y (438-455; 516-540) | 881 | At1g51790 | LRRI | 270 |
| | (light | | | | | |
| | repressible) | | | | | |
| 1.8-5 | LRR | Y (512-536; 561-585) | 863 | At4g29450 | LRRI | 268 |
| | (light | | | | | |
| | repressible) | | | | | |
| 1.8-5 | LRR | Y (6-22; 508-530; | 911 | At4g29180 | LRRI | 267 |
| | (light | 555-571) | | | | |
| | repressible) | | | | | |
| 1.8-6 | LRR | Y (6-23; 512-536; | 894 | At1g51800 | LRRI | 252 |
| | (light | 595-612) | | | | |
| | repressible) | | | | | |
| 1.8-6 | PK | Y (413-429; 460-483) | 837 | At1g51870 | LRRI | 254 |
| 1.8-6 | RLK (LRR- | Y (511-528; 589-606) | 890 | At1g51860 | LRRI | 253 |
| | I) | | | | | |
| 1.8-6 | LRR | Y (462-484; 517-541) | 880 | At1g51880 | LRRI | 255 |
| | (light | | | | | |
| | repressible) | | | | | |
| 1.8-6 | LRR | Y (490-514; 615-632) | 888 | At1g51890 | LRRI | 256 |
| | (light | | | | | |
| | repressible) | | | | | |
| 1.8-6 | PK | Y (6-23; 460-477; | 876 | At1g51910 | LRRI | 257 |
| | | 508-531) | | | | |
| 1.8-7 | LRR | Y (447-469; 506-529) | 884 | At2g28990 | LRRI | 242 |
| | (light | | | | | |
| | repressible) | | | | | |
| 1.8-7 | LRR | Y (408-432; 477-494) | 786 | At2g28970 | LRRI | 241 |
| | (light | | | | | |
| | repressible) | | | | | |
| 1.8-8 | LRR | Y (7-24) | 898 | At4g20450 | LRRI | 239 |
| | (light | | | | | |
| | repressible) | | | | | |
| 1.8-9 | LRR | Y (6-22; 510-529; | 872 | At2g29000 | N.A. | N.A. |
| | | 562-579) | | | | |
| 1.8-9 | LRR | Y (8-24; 509-532; | 880 | At2g28960 | LRRI | 237 |
| | (light | 578-594) | | | | |
| | repressible) | | | | | |
| 1.8-10 | LRR | Y (10-27; 464-483; | 880 | At3g21340 | LRRI | 250 |
| | (light | 519-543) | | | | |
| | repressible) | | | | | |
| 1.8-10 | LRR | Y (7-24; 518-542; | 888 | At1g49100 | LRRI | 251 |
| | (light | 579-596) | | | | |
| | repressible) | | | | | |
| 1.8-10 | LRR | Y (7-24; 479-503; | 851 | At2g04300 | LRRI | 249 |
| | (light | 539-556) | | | | |
| | repressible) | | | | | |
| 1.8-11 | LRR | Y (505-529; 572-591) | 884 | At1g51805 | N.A. | N.A. |
| | (light | | | | | |
| | repressible) | | | | | |
| 1.8-11 | LRR | N | 843 | At1g51810 | LRRI | 248 |
| | (light | | | | | |
| | repressible) | | | | | |
| 1.8-11 | LRR | Y (506-530; 573-592) | 885 | At1g51820 | LRRI | 244 |
| | (light | | | | | |
| | repressible) | | | | | |
| 1.8-11 | LRR | Y (486-510; 555-572) | 865 | At1g51850 | LRRI | 245 |
| | (light | | | | | |
| | repressible) | | | | | |
| 1.8-12 | LRR | Y (459-478; 503-522) | 868 | At5g59670 | LRRI | 236 |
| | (light | | | | | |
| | repressible) | | | | | |
| 1.8-12 | LRR | Y (8-27; 515-537; | 866 | At5g16900 | LRRI | 240 |
| | (light | 568-587) | | | | |
| | repressible) | | | | | |
| 1.8-12 | LRR | Y (6-23; 463-480; | 878 | At3g46330 | LRRI | 232 |
| | (light | 517-534) | | | | |
| | repressible) | | | | | |
| 1.8-12 | LRR | Y (362-378; 517-541) | 892 | At5g59650 | LRRI | 233 |
| 1.8-12 | LRR | Y (509-531) | 882 | At5g59680 | LRRI | 234 |
| | (light | | | | | |
| | repressible) | | | | | |
| 1.8-12 | LRR | Y (6-22; 462-481; | 856 | At1g07560 | LRRI | 243 |
| | (light | 496-520) | | | | |
| | repressible) | | | | | |
| 1.8-13 | LRR | Y (508-530; 561-578) | 868 | At2g14510 | LRRI | 258 |
| | (light | | | | | |
| | repressible) | | | | | |
| 1.8-13 | LRR | Y (506-527; 558-575) | 864 | At1g07550 | LRRI | 260 |
| | (light | | | | | |
| | repressible) | | | | | |
| 1.8-13 | LRR | Y (7-23; 526-548; | 886 | At2g14440 | LRRI | 259 |
| | (light | 579-595) | | | | |
| | repressible) | | | | | |
| 1.8-14 | LRR | Y (6-23; 452-469 | 889 | At3g46340 | LRRI | 226 |
| | (light | 511-535) | | | | |
| | repressible) | | | | | |
| 1.8-14 | LRR | N | 871 | At3g46350 | LRRI | 227 |
| 1.8-14 | LRR | N | 838 | At3g46420 | LRRI | 231 |
| 1.8-15 | LRR | Y (7-31; 508-532; | 883 | At3g46400 | LRRI | 230 |
| | (light | 581-598) | | | | |
| | repressible) | | | | | |
| 1.8-15 | LRR | Y (427-450; 492-509) | 793 | At3g46370 | LRRI | 229 |
| | (light | | | | | |
| | repressible) | | | | | |

Family
1.9

| | | | | | | |
|---|---|---|---|---|---|---|
| 1.9-1 | PK | Y (441-464; 530-553) | 880 | At5g38990 | CrRLK1L-1 | 208 |
| 1.9-1 | PK | Y (441-465; 522-546) | 873 | At5g39000 | CrRLK1L-1 | 209 |
| 1.9-2 | PK | Y (444-465; 496-513) | 806 | At5g39030 | CrRLK1L-2 | 129 |
| 1.9-2 | PK | Y (6-22; 438-462; | 813 | At5g39020 | CrRLK1L-2 | 128 |
| | | 475-491) | | | | |
| 1.9-3 | PR55K P | Y (11-28) | 579 | At5g38250 | LRK10L-2 | 132 |
| 1.9-3 | PR55K P | Y (14-30) | 588 | At5g38240 | LRK10L-2 | 131 |
| 1.9-4 | PR5K P | Y (465-484; 565-584) | 853 | At4g18250 | Thaumatin | 139 |
| 1.9-4 | PK | Y (744-763; 794-811) | 1109 | At1g66980 | LRK10L-2 | 135 |
| 1.9-4 | PR5K P | N | 799 | At1g70250 | Thaumatin | 140 |
| 1.9-4 | PR5K | Y (6-23; 277-297; | 665 | At5g38280 | Thaumatin | 138 |
| | | 329-346) | | | | |
| 1.9-5 | PK | Y (71-93; 133-155) | 470 | At5g24080 | SD-2 | 144 |
| 1.9-6 | RLK4 | N | 402 | At4g00340 | SD-2 | 142 |
| 1.9-7 | Lec | Y (11-35; 390-407; | 829 | At1g34300 | SD-2 | 146 |
| | Binding | 422-439) | | | | |
| | PK | | | | | |
| 1.9-7 | Lec | Y (6-23; 449-466; | 764 | At2g41890 | SD-3 | 602 |
| | Binding | 483-500) | | | | |
| | PK | | | | | |
| 1.9-7 | Lec | Y (6-23) | 748 | At5g60900 | SD-2 | 141 |
| | Binding | | | | | |
| | PK | | | | | |
| 1.9-8 | Lec | Y (6-25; 431-450; | 821 | At4g32300 | SD-2 | 145 |
| | Binding | 537-556) | | | | |
| | PK | | | | | |
| 1.9-8 | Lec | Y (6-25; 442-464; | 870 | At5g35370 | SD-2 | 147 |
| | Binding | 519-540) | | | | |
| | PK | | | | | |
| 1.9-9 | S-locus | Y (440-463; 494-512) | 828 | At2g19130 | SD-2 | 143 |
| | LecRK | | | | | |

Family
1.10

| | | | | | | |
|---|---|---|---|---|---|---|
| 1.10-1 | RLK | N | 756 | At1g21590 | LRR VI | 102 |
| 1.10-1 | RLK | N | 794 | At1g77280 | LRR VI | 101 |
| 1.10-1 | PK | N | 705 | At5g63940 | LRR VI | 103 |
| 1.10-2 | PK | N | 321 | At4g35030 | LRR VI | 104 |
| 1.10-2 | PK | N | 617 | At2g16750 | LRR VI | 105 |
| 1.10-3 | PK | N | 467 | At5g10520 | LRR VI | 111 |
| 1.10-3 | PK | Y (327-344) | 461 | At3g05140 | LRR VI | 110 |
| 1.10-3 | PK | N | 456 | At5g65530 | LRR VI | 112 |
| 1.10-3 | PK | Y (157-173) | 511 | At5g18910 | LRR VI | 109 |
| 1.10-4 | PK | N | 552 | At5g37790 | LRR VI | 106 |
| 1.10-4 | PK | N | 467 | At1g66460 | LRR VI | 107 |
| 1.10-5 | PK | N | 416 | At5g57670 | LRR VI | 114 |
| 1.10-6 | PK | Y (128-147) | 392 | At2g18890 | LRR VI | 113 |
| 1.10-7 | PK | N | 429 | At5g35960 | LRR VI | 108 |

Family
1.11

| | | | | | | |
|---|---|---|---|---|---|---|
| 1.11-1 | LecRK | Y (19-38; 284-308; | 675 | At5g65600 | L-Lectin | 533 |
| | | 407-424) | | | | |
| 1.11-1 | LecRK 3 P | Y (6-24; 269-292; | 651 | At5g10530 | L-Lectin | 532 |
| | | 344-363) | | | | |
| 1.11-2 | LecRK | Y (270-289; 326-345) | 652 | At5g06740 | L-Lectin | 534 |
| 1.11-3 | LecRK | Y (95-119; 314-338; | 711 | At5g03140 | L-Lectin | 529 |
| | | 369-393) | | | | |
| 1.11-3 | LecRK | Y (113-135; 307-331) | 691 | At5g42120 | L-Lectin | 531 |
| 1.11-3 | LecRK | Y (4-21; 82-106; | 715 | At3g53380 | L-Lectin | 528 |
| | | 316-339) | | | | |
| 1.11-3 | LecRK 3 L | Y (7-26; 306-325; | 681 | At5g55830 | L-Lectin | 530 |
| | | 374-393) | | | | |
| 1.11-4 | LecRK 3 L | Y (18-41; 72-89; | 686 | At4g04960 | L-Lectin | 527 |
| | | 287-310) | | | | |
| 1.11-4 | LecRK 3 L | Y (13-30; 302-325) | 656 | At1g15530 | L-Lectin | 507 |
| 1.11-4 | LecRK | Y (6-24; 37-55; | 649 | At4g28350 | L-Lectin | 526 |
| | | 76-94) | | | | |
| 1.11-5 | PK | Y (6-22; 296-313; | 675 | At2g37710 | L-Lectin | 502 |
| | | 351-368) | | | | |
| 1.11-5 | LecRK 3 P | Y (7-25; 240-261; | 677 | At3g53810 | L-Lectin | 503 |
| | | 292-313) | | | | |
| 1.11-6 | LecRK | Y (6-23; 38-55; | 674 | At4g02410 | L-Lectin | 504 |
| | | 86-103; 248-265; | | | | |
| | | 298-317) | | | | |
| 1.11-6 | LecRK 3 L | Y (6-23; 40-59; | 669 | At4g02420 | L-Lectin | 505 |
| | | 90-109; 245-264; | | | | |
| | | 295-312) | | | | |
| 1.11-7 | LecRK | Y (253-270; 287-311) | 669 | At4g29050 | L-Lectin | 500 |
| 1.11-7 | LecRK 3 L | Y (7-23; 228-245; | 656 | At1g70130 | L-Lectin | 499 |
| | | 277-301) | | | | |
| 1.11-7 | LecRK | Y (233-256; 287-311) | 666 | At1g70110 | L-Lectin | 501 |
| 1.11-7 | LecRK 3 P | Y (7-31; 75-93; | 684 | At3g55550 | L-Lectin | 506 |
| | | 236-254; 289-313) | | | | |
| 1.11-8 | LecRK | Y (102-125; 293-315) | 668 | At5g59270 | L-Lectin | 524 |
| 1.11-8 | LecRK 3 P | Y (6-23; 305-322; | 674 | At5g59260 | L-Lectin | 523 |
| | | 360-377) | | | | |
| 1.11-9 | LecRK 3 L | Y (7-23; 69-93; | 623 | At2g29250 | L-Lectin | 536 |
| | | 241-263; 303-327) | | | | |
| 1.11-9 | LecRK 3 L | Y (6-23; 244-261; | 627 | At2g29220 | L-Lectin | 537 |
| | | 304-321) | | | | |
| 1.11- | LecRK | Y (236-258; 289-308) | 718 | At5g60300.1 | L-Lectin | 520 |
| 10 | | | | | | |
| 1.11- | LecRK | Y (236-258; 289-308) | 718 | At5g60300.2 | L-Lectin | 520 |
| 10 | | | | | | |
| 1.11- | LecRK | Y (233-255; 286-305) | 668 | At5g60270 | L-Lectin | 522 |
| 10 | | | | | | |
| 1.11- | LecRK | Y (7-24; 241-262; | 682 | At3g45330 | L-Lectin | 512 |
| 10 | | 293-310) | | | | |
| 1.11- | LecRK | Y (6-23; 296-317; | 604 | At3g45390 | L-Lectin | 513 |
| 10 | | 425-442) | | | | |
| 1.11- | LecRK | Y (234-256; 287-306) | 669 | At3g45440 | L-Lectin | 517 |
| 10 | | | | | | |
| 1.11- | LecRK | Y (240-262; 293-312) | 675 | At5g60320 | L-Lectin | 514 |
| 10 | | | | | | |
| 1.11- | LecRK | Y (234-256; 281-303) | 657 | At5g60280 | L-Lectin | 518 |
| 10 | | | | | | |
| 1.11- | LecRK | Y (234-256; 287-306) | 664 | At3g45410 | L-Lectin | 515 |
| 10 | | | | | | |
| 1.11- | LecRK | Y (296-315; 346-365) | 667 | At3g45420 | L-Lectin | 516 |
| 010 | | | | | | |
| 1.11- | LecRK | Y (174-195; 226-247) | 613 | At3g45430 | L-Lectin | 519 |
| 010 | | | | | | |
| 1.11- | LecRK 3 P | Y (235-257; 288-307) | 616 | At5g60310 | L-Lectin | 521 |
| 010 | | | | | | |
| 1.11- | LecRK 3 P | Y (7-24; 85-102; | 682 | At5g01540 | L-Lectin | 510 |
| 011 | | 310-333; 360-377) | | | | |
| 1.11- | LecRK 3 P | Y (311-335; 373-395) | 693 | At3g08870 | L-Lectin | 511 |
| 011 | | | | | | |
| 1.11- | LecRK 3 P | Y (306-330) | 691 | At5g01560 | L-Lectin | 509 |
| 011 | | | | | | |
| 1.11- | LecRK 3 P | Y (304-328) | 688 | At5g01550 | L-Lectin | 508 |
| 011 | | | | | | |
| 1.11- | LecRK 3 P | Y (227-246; 277-300) | 523 | At3g59730 | L-Lectin | 496 |
| 012 | | | | | | |
| 1.11- | LecRK1 P | Y (7-25; 234-257; | 664 | At2g43690 | L-Lectin | 498 |
| 012 | | 278-301) | | | | |
| 1.11- | LecRK | Y (278-302; 339-356) | 658 | At2g43700 | L-Lectin | 497 |
| 012 | | | | | | |
| 1.11- | LecRK | Y (27-44; 65-82; | 661 | At3g59700 | L-Lectin | 495 |
| 012 | | 238-255; 280-304) | | | | |
| 1.11- | LecRK 3 P | Y (248-265; 276-300) | 659 | At3g59740 | L-Lectin | 493 |
| 012 | | | | | | |
| 1.11- | LecRK 3 P | Y (196-215; 246-268) | 626 | At3g59750 | L-Lectin | 494 |
| 012 | | | | | | |
| 1.11- | PK | N | 337 | At3g46760 | L-Lectin | 525 |
| 013 | | | | | | |

Family
1.12

| | | | | | | |
|---|---|---|---|---|---|---|
| 1.12-1 | PK | Y (11-29; 131-148) | 355 | At1g78530 | LRR XIII | 378 |
| 1.12-2 | PK | Y (9-27; 62-84) | 376 | At5g13290.1 | N.A. | 336 |
| 1.12-2 | PK | Y (9-27; 62-84) | 331 | At5g13290.2 | N.A. | 336 |
| 1.12-3 | RPK1 | Y (199-220; 241-261) | 540 | At1g69270 | N.A. | 287 |
| 1.12-4 | LRR | Y (15-32; 285-309) | 641 | At2g31880 | LRR XI | 374 |
| 1.12-5 | LRR | Y (11-29; 230-247; | 605 | At3g28450 | LRR X | 386 |
| | | 297-314) | | | | |
| 1.12-5 | CLV1 P | Y (4-21; 221-245) | 601 | At1g27190 | LRR X | 385 |
| 1.12-5 | LRR | Y (215-239; 351-368) | 591 | At1g69990 | LRR X | 384 |
| 1.12-5 | LRR | Y (6-25; 227-246; | 620 | At5g48380 | LRR X | 387 |
| | | 358-375) | | | | |
| 1.12-6 | IMK2 | Y (18-35; 459-483) | 836 | At3g51740 | LRR III | 328 |
| 1.12-6 | MRLK | Y (373-395; 533-555; | 719 | At3g56100 | LRR III | 329 |
| | | 572-589) | | | | |
| 1.12-7 | LRR | Y (646-667; 686-705) | 985 | At3g02130 | N.A. | |
| 1.12-8 | LRR | Y (512-536; 557-574) | 882 | At1g12460 | LRR VII | 346 |
| 1.12-8 | LRR | Y (6-22; 518-540; | 890 | At1g62950 | LRR VII | 345 |
| | | 605-627) | | | | |
| 1.12-9 | LRR | N | 1036 | At5g53890 | LRR X | 393 |
| 1.12-9 | LRR | Y (20-44) | 1095 | At1g72300 | LRR X | 395 |
| 1.12-9 | LRR | N | 1008 | At2g02220 | LRR X | 394 |
| 1.12- | LRR | Y (20-37) | 966 | At1g34420 | LRR X | 383 |
| 10 | | | | | | |
| 1.12- | LRR | Y (9-28; 541-560; | 872 | At5g06940 | N.A. | 601 |
| 10 | | 645-662) | | | | |
| 1.12- | RLK | Y (535-558) | 890 | At2g41820 | LRR X | 382 |
| 10 | | | | | | |
| 1.12- | LRR | Y (6-24) | 1133 | At1g17230 | LRR XI | 353 |
| 11 | | | | | | |
| 1.12- | LRR | Y (14-30; 707-725; | 1124 | At2g33170 | LRR XI | 351 |
| 11 | | 753-772) | | | | |
| 1.12- | LRR | Y (8-27) | 1102 | At5g63930 | LRR XI | 352 |
| 11 | | | | | | |
| 1.12- | LRR | Y (7-26) | 953 | At5g56040 | LRR XI | 357 |
| 12 | | | | | | |
| 1.12- | LRR | N | 1045 | At1g34110 | LRR XI | 358 |
| 12 | | | | | | |
| 1.12- | CLV1 L | Y (7-28) | 1141 | At3g24240 | LRR XI | 355 |
| 12 | | | | | | |
| 1.12- | LRR | Y (12-31) | 1135 | At5g48940 | LRR XI | 354 |
| 12 | | | | | | |
| 1.12- | PK | Y (8-25) | 1089 | At4g26540 | LRR XI | 356 |
| 12 | | | | | | |
| 1.12- | LRR | Y (6-29; 770-793; | 1123 | At1g73080 | LRR XI | 372 |
| 13 | | 831-848) | | | | |
| 1.12- | InRPK P | Y (738-761) | 1088 | At1g17750 | LRR XI | 373 |
| 13 | | | | | | |
| 1.12- | LRR | Y (30-48; 709-727) | 1045 | At4g08850.1 | LRR XII | 551 |
| 14 | | | | | | |
| 1.12- | LRR | Y (30-47; 709-726; | 1009 | At4g08850.2 | LRR XII | 551 |
| 14 | | 991-1008) | | | | |
| 1.12- | LRR | Y (13-32) | 1120 | At1g35710 | LRR XII | 552 |
| 14 | | | | | | |
| 1.12- | LRR | Y (875-894; | 1249 | At4g20140 | LRR XI | 367 |
| 15 | | 1008-1026) | | | | |
| 1.12- | LRR | Y (875-898; | 1252 | At5g44700 | LRR XI | 368 |
| 15 | | 1005-1023) | | | | |
| 1.12- | FLS2 | Y (7-23; 807-823; | 1173 | At5g46330 | LRR XII | 550 |
| 15 | | 869-885) | | | | |
| 1.12- | EMS1 | Y (827-846) | 1192 | At5g07280 | LRR X | 392 |
| 15 | | | | | | |
| 1.12- | LRR | Y (753-772) | 1136 | At4g36180 | LRR VII | 340 |
| 16 | | | | | | |
| 1.12- | LRR | Y (484-503; 754-772; | 1140 | At1g75640 | LRR VII | 341 |
| 16 | | 943-961) | | | | |
| 1.12- | LRR | Y (609-629) | 976 | At1g09970.1 | LRR XI | 369 |
| 17 | | | | | | |
| 1.12- | LRR | Y (609-629) | 977 | At1g09970.2 | LRR XI | 369 |
| 17 | | | | | | |
| 1.12- | IKU2 | Y (448-465; 616-635) | 991 | At3g19700 | LRR XI | 370 |
| 17 | | | | | | |
| 1.12- | LRR | Y (7-24; 624-641; | 977 | At1g72180 | LRR XI | 365 |
| 17 | | 743-760) | | | | |
| 1.12- | LRR | Y (624-641) | 996 | At1g28440 | LRR XI | 363 |
| 18 | | | | | | |
| 1.12- | HAESA/RLK5 | Y (625-648) | 999 | At4g28490 | LRR XI | 362 |
| 18 | | | | | | |
| 1.12- | LRR | Y (633-653) | 993 | At5g65710 | LRR XI | 364 |
| 18 | | | | | | |
| 1.12- | Pre RLK5 | Y (628-650; 681-704) | 1005 | At5g25930 | LRR XI | 371 |
| 18 | | | | | | |
| 1.12- | LRR | Y (6-23; 594-611; | 966 | At5g49660 | LRR XI | 366 |
| 18 | | 721-738) | | | | |
| 1.12- | BAM1 | Y (642-661) | 1003 | At5g65700 | LRR XI | 347 |
| 19 | | | | | | |
| 1.12- | LRR | Y (589-613; 678-701) | 895 | At5g51350 | LRR IV | 608 |
| 19 | | | | | | |
| 1.12- | LRR | Y (585-604; 635-658) | 960 | At2g25790 | N.A. | 600 |
| 19 | | | | | | |
| 1.12- | CLV1 | Y (641-659; 749-766) | 980 | At1g75820 | LRR XI | 349 |
| 19 | | | | | | |
| 1.12- | BAM3 | Y (6-24; 659-678; | 992 | At4g20270 | LRR XI | 350 |
| 19 | | 767-784) | | | | |
| 1.12- | BAM2 | Y (638-657; 748-765) | 1002 | At3g49670 | LRR XI | 348 |
| 19 | | | | | | |
| 1.12- | CLV1 L | Y (6-23; 649-666; | 1029 | At1g08590 | LRR XI | 360 |
| 20 | | 697-714) | | | | |
| 1.12- | RPK5 | Y (6-25; 634-653; | 1013 | At4g28650 | LRR XI | 359 |
| 20 | | 682-699) | | | | |
| 1.12- | PXY RLK | Y (6-25) | 1041 | At5g61480 | LRR XI | 361 |
| 20 | | | | | | |
| 1.12- | LRR Xa21 | Y (601-619; 642-666) | 1010 | At3g47570 | LRR XII | 545 |
| 21 | | | | | | |
| 1.12- | LRR Xa21 | Y (71-93; 643-665; | 1009 | At3g47090 | LRR XII | 544 |
| 21 | | 696-715) | | | | |
| 1.12- | LRR Xa21 | Y (643-667; 698-722) | 1011 | At3g47580 | LRR XII | 543 |
| 21 | | | | | | |
| 1.12- | LRR Xa21 | Y (654-678; 715-734) | 1025 | At3g47110 | LRR XII | 548 |
| 21 | | | | | | |
| 1.12- | LRR Xa21 | Y (605-624; 651-670) | 1031 | At5g20480 | LRR XII | 546 |
| 21 | | | | | | |
| 1.12- | ERL1 | Y (8-26; 556-574; | 966 | At5g62230 | LRR XIII | 380 |
| 22 | | 585-603) | | | | |
| 1.12- | LRR | N | 980 | At2g24130 | LRR XII | 549 |
| 22 | | | | | | |
| 1.12- | ER | Y (7-23; 551-567; | 976 | At2g26330 | LRR XIII | 381 |
| 22 | | 582-599) | | | | |
| 1.12- | ERL2 | Y (523-542; 551-570) | 932 | At5g07180 | LRR XIII | 379 |
| 22 | | | | | | |
| 1.12- | LRPKm1 | Y (606-630; 749-766; | 967 | At5g01890 | LRR VII | 342 |
| 23 | | 787-805) | | | | |
| 1.12- | LRPKm | Y (598-622; 740-757) | 964 | At3g56370 | LRR VII | 343 |
| 23 | | | | | | |
| 1.12- | LRR | Y (7-24; 643-667; | 1016 | At3g28040 | LRR VII | 344 |
| 23 | | 784-801) | | | | |
| 1.12- | BRL3 | Y (773-796) | 1164 | At3g13380 | LRR X | 389 |
| 24 | | | | | | |
| 1.12- | BRL P | Y (17-34) | 1106 | At1g74360 | LRR X | 397 |
| 24 | | | | | | |
| 1.12- | BRI1 | Y (6-24; 792-811) | 1196 | At4g39400 | LRR X | 390 |
| 24 | | | | | | |
| 1.12- | BRL1 | Y (6-22; 774-797) | 1166 | At1g55610 | LRR X | 388 |
| 24 | | | | | | |
| 1.12- | BRL P | Y (757-776) | 1143 | At2g01950 | LRR X | 391 |
| 24 | | | | | | |
| 1.12- | LRR | Y (13-30) | 648 | At4g30520 | LRR II | 158 |
| 25 | | | | | | |
| 1.12- | LRR | Y (6-23; 234-258; | 634 | At2g23950 | LRR II | 157 |
| 25 | | 292-309) | | | | |
| 1.12- | LRR | Y (11-28; 247-270) | 635 | At3g25560.1 | LRR II | 159 |
| 26 | | | | | | |
| 1.12- | LRR | Y (11-28; 248-271) | 636 | At3g25560.2 | LRR II | 159 |
| 26 | | | | | | |
| 1.12- | LRR | Y (11-28; 237-259) | 632 | At1g60800 | LRR II | 161 |
| 26 | | | | | | |
| 1.12- | LRR | Y (14-33; 247-269) | 638 | At5g16000 | LRR II | 160 |
| 26 | | | | | | |
| 1.12- | LRR | Y (8-27; 240-264; | 614 | At5g45780 | LRR II | 162 |
| 26 | | 295-314) | | | | |
| 1.12- | SERK1 | Y (235-259) | 625 | At1g71830 | LRR II | 150 |
| 27 | | | | | | |
| 1.12- | SERK2 | Y (8-27; 239-262) | 628 | At1g34210 | LRR II | 149 |
| 27 | | | | | | |
| 1.12- | SERKL4 | Y (10-29) | 620 | At2g13790 | LRR II | 152 |
| 27 | | | | | | |
| 1.12- | SERK3 | Y (223-246) | 615 | At4g33430 | LRR II | 151 |
| 27 | (BAK1) | | | | | |
| 1.12- | SERKL5 | Y (120-139; 217-236) | 601 | At2g13800 | LRR II | 153 |
| 27 | | | | | | |
| 1.12- | LRR | Y (9-26; 226-247) | 613 | At5g10290 | LRR II | 155 |
| 28 | | | | | | |
| 1.12- | RLK | Y (220-241) | 617 | At5g65240 | LRR II | 154 |
| 28 | | | | | | |
| 1.12- | LRR | Y (30-47) | 614 | At5g63710 | LRR II | 156 |
| 29 | | | | | | |
| 1.12- | LRR | Y (7-31; 241-265) | 604 | At5g62710 | LRR XIII | 377 |
| 30 | | | | | | |
| 1.12- | LRR | Y (239-263) | 592 | At1g31420 | LRR XIII | 375 |
| 30 | | | | | | |
| 1.12- | SERK1 P | Y (237-261; 272-288) | 589 | At2g35620 | LRR XIII | 376 |
| 30 | | | | | | |

Family
1.13

| | | | | | | |
|---|---|---|---|---|---|---|
| 1.13-1 | LRR | Y (270-293) | 672 | At2g36570 | LRR III | 322 |
| 1.13-2 | LRR | N | 669 | At5g67200 | LRR III | 297 |
| 1.13-2 | LRR | Y (275-299; 433-450) | 670 | At1g68400 | LRR III | 323 |
| 1.13-2 | LRR | Y (251-275) | 652 | At1g60630 | LRR III | 301 |
| 1.13-2 | LRR | Y (7-24; 280-302) | 669 | At5g43020 | LRR III | 299 |
| 1.13-2 | RKL1 P | Y (5-29; 293-317) | 660 | At3g50230 | LRR III | 298 |
| 1.13-3 | LRR | Y (261-285) | 640 | At3g08680.1 | LRR III | 313 |
| 1.13-3 | LRR | Y (261-285) | 640 | At3g08680.2 | LRR III | 313 |
| 1.13-3 | LRR | Y (257-281) | 658 | At2g26730 | LRR III | 315 |
| 1.13-3 | RLK | Y (21-45; 76-100; | 654 | At5g58300 | LRR III | 314 |
| | | 281-305) | | | | |
| 1.13-3 | LRR | Y (6-25; 222-241; | 640 | At5g05160 | LRR III | 321 |
| | | 266-290) | | | | |
| 1.13-4 | LRR | Y (7-24; 251-275; | 627 | At3g02880 | LRR III | 325 |
| | | 391-408) | | | | |
| 1.13-4 | LRR | Y (244-268) | 625 | At5g16590 | LRR III | 324 |
| 1.13-4 | RKL1 | Y (268-291) | 655 | At1g48480 | LRR III | 326 |
| 1.13-4 | RLK902 | Y (265-288) | 647 | At3g17840 | LRR III | 327 |
| 1.13-5 | RKL1 P | Y (258-282) | 638 | At4g23740 | LRR III | 316 |
| 1.13-5 | LRR | N | 587 | At1g64210 | LRR III | 318 |
| 1.13-5 | LRR | Y (7-24; 252-276; | 614 | At5g24100 | LRR III | 320 |
| | | 325-344) | | | | |
| 1.13-5 | LRR | Y (235-259) | 601 | At5g53320 | LRR III | 319 |
| 1.13-6 | PRK1 P | Y (8-25; 269-287; | 659 | At5g20690 | LRR III | 295 |
| | | 351-370) | | | | |
| 1.13-6 | PRK1 P | Y (251-270; 367-385) | 633 | At3g42880 | LRR III | 294 |
| 1.13-7 | LRR | Y (23-43; 166-188; | 686 | At1g50610 | LRR III | 292 |
| | | 280-304) | | | | |
| 1.13-7 | LRR | Y (9-26; 210-229; | 676 | At4g31250 | LRR III | 293 |
| | | 244-268) | | | | |
| 1.13-7 | LRR | Y (253-272) | 644 | At1g72460 | LRR III | 296 |
| 1.13-7 | LRR | Y (20-38; 172-196; | 679 | At3g20190 | LRR III | 291 |
| | | 278-302) | | | | |
| 1.13-7 | LRR | Y (245-267) | 647 | At2g07040 | LRR III | 290 |
| 1.13-7 | PRK1 | Y (9-26; 257-276; | 657 | At5g35390 | LRR III | 289 |
| | | 362-379) | | | | |
| 1.13-8 | LRR | N | 680 | At5g51560 | LRR IV | 491 |
| 1.13-8 | LRR | Y (5-22; 77-94; | 691 | At2g45340 | LRR IV | 490 |
| | | 311-328; 489-505) | | | | |
| 1.13-8 | LRR | Y (12-331; 607-626) | 688 | At4g22730 | LRR IV | 492 |
| 1.13-9 | LRR | Y (6-25; 280-299; | 662 | At3g57830 | LRR III | 306 |
| | | 365-386) | | | | |
| 1.13-9 | LRR | Y (276-298) | 646 | At2g42290 | LRR III | 305 |
| 1.13- | LRR | Y (14-38; 336-360) | 751 | At5g67280 | LRR III | 310 |
| 10 | | | | | | |
| 1.13- | LRR | Y (336-358) | 744 | At2g15300 | LRR III | 311 |
| 10 | | | | | | |
| 1.13- | LRR | Y (339-363) | 757 | At4g34220 | LRR III | 312 |
| 10 | | | | | | |
| 1.13- | LRR | Y (9-31; 333-355) | 773 | At2g23300 | LRR III | 308 |
| 10 | | | | | | |
| 1.13- | LRR | Y (329-352) | 768 | At4g37250 | LRR III | 309 |
| 10 | | | | | | |
| 1.13- | LRR | Y (317-336; 609-628) | 702 | At1g25320 | LRR III | 302 |
| 11 | | | | | | |
| 1.13- | LRR | Y (315-339) | 719 | At1g67510 | LRR III | 307 |
| 11 | | | | | | |
| 1.13- | LRR | N | 716 | At2g01210 | LRR III | 303 |
| 11 | | | | | | |
| 1.13- | LRR | Y (305-329) | 685 | At1g66830 | LRR III | 304 |
| 11 | | | | | | |
| 1.13- | RHG1 P | N | 359 | At5g41680.1 | LRR III | 317 |
| 12 | | | | | | |
| 1.13- | RHG1 P | N | 333 | At5g41680.2 | LRR III | 317 |
| 12 | | | | | | |

Family
1.14

| | | | | | | |
|---|---|---|---|---|---|---|
| 1.14-1 | PK | N | 351 | At4g11890.1 | DUF26 | 489 |
| 1.14-1 | PK | N | 352 | At4g11890.2 | DUF26 | 489 |
| 1.14-1 | PK | Y (21-38) | 354 | At4g11890.3 | DUF26 | 489 |
| 1.14-2 | PK | N | 341 | At5g23170 | CR4L | 85 |
| 1.14-3 | PK | Y (262-280) | 470 | At1g28390 | CR4L | 83 |
| 1.14-3 | PK | N | 362 | At3g51990 | CR4L | 84 |
| 1.14-4 | PK | Y (571-588) | 697 | At1g72760 | RLCK IX | 562 |
| 1.14-4 | PK | Y (97-114; 474-491; | 733 | At1g17540 | RLCK IX | 563 |
| | | 610-627) | | | | |
| 1.14-5 | PK | Y (432-451; 555-574) | 680 | At1g78940 | RLCK IX | 559 |
| 1.14-5 | PK | N | 758 | At1g16760 | RLCK IX | 560 |
| 1.14-5 | PK | N | 780 | At3g20200 | RLCK IX | 561 |
| 1.14-6 | PK | N | 731 | At5g35380 | RLCK IX | 557 |
| 1.14-6 | PK | N | 700 | At2g07020 | RLCK IX | 558 |
| 1.14-6 | PK | N | 816 | At2g24370 | RLCK IX | 553 |
| 1.14-7 | PK | N | 703 | At5g26150 | RLCK IX | 555 |
| 1.14-7 | PK | Y (99-116; 560-577; | 703 | At5g12000 | RLCK IX | 556 |
| | | 608-627) | | | | |
| 1.14-8 | PnPK1 L | N | 845 | At5g61550 | RLCK IX | 566 |
| 1.14-8 | PK | Y (533-550) | 835 | At4g25160 | RLCK IX | 564 |
| 1.14-8 | PK | Y (513-530) | 819 | At5g51270 | RLCK IX | 565 |
| 1.14-8 | PK | N | 796 | At5g61560 | RLCK IX | 567 |
| 1.14-9 | U-Box PK | Y (59-81) | 801 | At2g19410 | RLCK IX | 568 |
| 1.14- | U-Box PK | N | 834 | At2g45910 | RLCK IX | 569 |
| 10 | | | | | | |
| 1.14- | U-Box PK | N | 805 | At3g49060 | RLCK IX | 570 |
| 10 | | | | | | |
| 1.14- | U-Box PK | N | 765 | At5g65500 | RLCK IX | 571 |
| 10 | | | | | | |

Family
1.15

| | | | | | | |
|---|---|---|---|---|---|---|
| 1.15-1 | PK | Y (145-168) | 499 | At3g56050 | RLCK I | 579 |
| 1.15-1 | PK | Y (7-24; 143-160) | 489 | At2g40270.1 | RLCK I | 578 |
| 1.15-1 | PK | Y (7-24; 136-153) | 482 | At2g40270.2 | RLCK I | 578 |
| 1.15-2 | LRR | Y (13-32; 230-253) | 553 | At5g07150 | LRR VI | 575 |
| 1.15-2 | RLK | Y (8-26; 320-343) | 686 | At4g18640 | LRR VI | 576 |
| 1.15-2 | LRR | Y (151-167; 312-334) | 668 | At5g45840 | LRR VI | 577 |
| 1.15-3 | PK | Y (142-166) | 484 | At5g58540.1 | RLCK I | 574 |
| 1.15-3 | PK | N | 242 | At5g58540.2 | RLCK I | 574 |
| 1.15-3 | PK | Y (6-23) | 341 | At5g58540.3 | RLCK I | 574 |
| 1.15-4 | LRR | Y (388-412; 533-557; | 802 | At3g03770 | LRR VI | 584 |
| | | 584-606) | | | | |
| 1.15-4 | LRR | Y (103-127; 396-420) | 812 | At5g14210 | LRR VI | 585 |
| 1.15-4 | LRR | Y (9-25; 300-316; | 747 | At1g14390 | LRR VI | 582 |
| | | 354-377) | | | | |
| 1.15-4 | LRR | Y (301-317; 354-377) | 753 | At2g02780 | LRR VI | 583 |
| 1.15-4 | LRR-VI | N | 680 | At5g63410 | LRR VI | 586 |
| 1.15-5 | ER P | Y (417-440) | 864 | At4g39270.1 | LRR IV | 606 |
| 1.15-5 | ER P | Y (417-440) | 694 | At4g39270.2 | LRR IV | 606 |
| 1.15-5 | LRR | Y (8-27; 447-471) | 915 | At2g16250 | N.A. | 607 |
| 1.15-6 | LRR | Y (13-30; 282-299) | 664 | At5g41180 | LRR VI | 581 |
| 1.15-6 | LRR | Y (6-24) | 664 | At1g63430 | LRR VI | 580 |

Family
1.16

| | | | | | | |
|---|---|---|---|---|---|---|
| 1.16-1 | PK | Y (19-36) | 422 | At1g63500 | N.A. | N.A. |
| 1.16-2 | PK | N | 489 | At4g00710 | N.A. | N.A. |
| 1.16-2 | PK | N | 483 | At1g01740 | N.A. | N.A. |
| 1.16-2 | PK | N | 487 | At5g41260 | N.A. | N.A. |
| 1.16-2 | PK | N | 489 | At5g46570 | N.A. | N.A. |
| 1.16-3 | PK | N | 490 | At3g54030 | N.A. | N.A. |
| 1.16-3 | PK | N | 507 | At1g50990 | N.A. | N.A. |
| 1.16-3 | PK | N | 477 | At3g09240 | N.A. | N.A. |
| 1.16-3 | PK | N | 499 | At5g01060 | RLCK II | 590 |
| 1.16-3 | PK | N | 489 | At5g59010 | RLCK II | 589 |
| 1.16-3 | PK | N | 512 | At4g35230 | RLCK II | 588 |
| 1.16-4 | PK | N | 465 | At2g17090 | RLCK II | 591 |
| 1.16-4 | PK | N | 328 | At2g17170 | N.A. | N.A. |

Family
1.17

| | | | | | | |
|---|---|---|---|---|---|---|
| 1.17-1 | PK | N | 269 | At3g57770 | RLCK III | 597 |
| 1.17-2 | PK | Y (177-194; 258-275) | 355 | At3g57730 | N.A. | N.A. |
| 1.17-2 | PK | Y (253-272) | 351 | At3g57710 | RLCK III | 596 |
| 1.17-2 | PK | Y (70-89) | 359 | At3g57720 | RLCK III | 595 |
| 1.17-2 | PK | N | 334 | At3g57750.1 | N.A. | N.A. |
| 1.17-2 | PK | N | 334 | At3g57750.2 | N.A. | N.A. |

No
Family

| | | | | | | |
|---|---|---|---|---|---|---|
| No | PK | N | 342 | At4g10390 | N.A. | 610 |
| Fam-1 | | | | | | |
| No | PK RLK | Y (48-70) | 349 | At1g33260.1 | N.A. | 609 |
| Fam-1 | | | | | | |
| No | PK RLK | Y (48-70) | 348 | At1g33260.2 | N.A. | 609 |
| Fam-1 | | | | | | |
| No | PK | N | 389 | At1g67470 | RLCK III | 598 |
| Fam-2 | | | | | | |
| No | PK | Y (225-241) | 372 | At1g65250 | RLCK III | 599 |
| Fam-2 | | | | | | |
| No | PK | N | 356 | At3g57640 | N.A. | |
| Fam-2 | | | | | | |
| No | PK | Y (7-26; 35-52; | 418 | At4g32000 | RLCK X | 274 |
| Fam-3 | | 65-84) | | | | |
| No | PK | Y (7-23; 71-94) | 427 | At1g80640 | RLCK X | 271 |
| Fam-3 | | | | | | |
| No | PK | Y (15-34) | 383 | At2g25220 | RLCK X | 273 |
| Fam-3 | | | | | | |
| No | PK | Y (6-25) | 372 | At5g11020 | RLCK X | 272 |
| Fam-3 | | | | | | |
| No | PK | Y (23-44) | 492 | At1g56720.1 | TAKL | 118 |
| Fam-4 | | | | | | |
| No | PK | Y (23-44) | 492 | At1g56720.2 | TAKL | 118 |
| Fam-4 | | | | | | |
| No | PK | Y (9-31) | 466 | At1g09440 | TAKL | 117 |
| Fam-4 | | | | | | |
| No | PK | Y (31-51) | 683 | At2g45590 | RLCK XI | 279 |
| Fam-5 | | | | | | |
| No | PK | Y (41-60) | 651 | At4g25390.1 | RLCK XI | 278 |
| Fam-5 | | | | | | |
| No | PK | Y (41-60) | 497 | At4g25390.2 | RLCK XI | 278 |
| Fam-5 | | | | | | |
| No | PK | Y (31-50) | 654 | At5g51770 | RLCK XI | 277 |
| Fam-5 | | | | | | |
| No | RKF1 P | Y (8-32; 576-593; | 1006 | At1g29750.1 | LRR | 474 |
| Fam-6 | | 606-630; | | | VIII-2 | |
| | | 856-880) | | | | |
| No | RKF1 P | Y (21-40; 591-608; | 1021 | At1g29750.2 | LRR | 474 |
| Fam-6 | | 621-645; | | | VIII-2 | |
| | | 868-887) | | | | |
| No | LRR | Y (427-444; 457-476) | 853 | At2g24230 | LRR VII | 337 |
| Fam-7 | | | | | | |
| No | LRR | Y (437-459; 532-551) | 785 | At5g58150 | LRR VII | 338 |
| Fam-7 | | | | | | |
| No | CRK13 | Y (6-24; 226-243; | 610 | At4g23210.1 | DUF26 | 422 |
| Fam-8 | | 302-320) | | | | |
| No | CRK13 | Y (6-24; 226-243; | 524 | At4g23210.2 | DUF26 | 422 |
| Fam-8 | | 302-320) | | | | |
| No | CRK11 | Y (6-23; 290-308; | 667 | At4g23190 | DUF26 | 401 |
| Fam-8 | | 395-412; | | | | |
| | | 616-633) | | | | |
| No | PK | N | 609 | At1g66920 | LRK10L-2 | 133 |
| Fam-9 | | | | | | |
| No | PK | Y (19-41; 573-595) | 692 | At1g80870 | RLCK XI | 280 |
| Fam-10 | | | | | | |
| No | PK | Y (37-61) | 458 | At1g54820 | Extensin | 80 |
| Fam-11 | | | | | | |
| No | PK | N | 270 | At3g21450 | RLCK IX | 573 |
| Fam-11 | | | | | | |
| No | PK | Y (300-319) | 674 | At3g24660 | LRR III | 332 |
| Fam-12 | | | | | | |

*The sequences associated with the AGI accession numbers are incorporated herein by reference.

TABLE 2
Phenotypes for all DN-RLK mutants generated grown on soil and on other
growth media in the T₂ and T₃ homozygous generations.

| | | | | | Confirmed |
|---|---|---|---|---|---|
| | DN-RLK | T₂ | Preliminary | T₃ | Phenotypes |
| RLK | Construct | Transgenic | Phenotypes | Homozygous | (various growth |
| Subfamily | (AGI) | Lines | (soil grown) | Lines | media) |
| 1.Other-9 | At4g20790 | 14 | none | 4 | shorter |
| | | | | | roots/less |
| | | | | | lateral roots* |
| | | | | | on MS |
| 1.Other- | At5g39390 | 17 | None | 2 | none |
| 10 | | | | | |
| 1.Other- | At5g45800 | 18 | senescent | 3 | none |
| 11 | | | leaves with | | |
| | | | more | | |
| | | | serrations/ | | |
| | | | stunted plant | | |
| | | | height/long | | |
| | | | skinny | | |
| | | | cauline | | |
| | | | leaves | | |
| 1.Other- | At5g10020 | 7 | none | 2 | longer roots on |
| 12 | | | | | MS/longer |
| | | | | | roots on- |
| | | | | | sucrose media |
| 1.Other- | At2g46850 | 3 | none | 2 | none |
| 13 | | | | | |
| 1.1-2 | At3g21630 | 18 | short stem/ | 2 | none |
| | | | larger leaves | | |
| 1.1-4 | At3g14350 | 13 | larger leaves | 4 | more lateral |
| | | | | | roots*/larger |
| | | | | | epidermal |
| | | | | | cells/ |
| | | | | | increased |
| | | | | | cellulose |
| | | | | | content |
| 1.1-6 | At5g06820 | 12 | short stem/ | 2 | short stem/ |
| | | | narrow leaves | | narrow leaves |
| 1.1-7 | At4g03390 | 3 | none | 1 | none |
| 1.2-28 | At5g01020 | 12 | none | 2 | none |
| 1.2-29 | At2g20300 | 16 | none | 8 | none |
| 1.2-31 | At2g28250 | 6 | longer | 3 | longer |
| | | | flowering | | flowering time |
| | | | time | | |
| 1.3-2 | At1g49730 | 6 | none | 3 | none |
| 1.3-4 | At5g59700 | 3 | none | 3 | short roots on |
| | | | | | MS, short roots |
| | | | | | on-sucrose |
| | | | | | media |
| 1.3-5 | At2g21480 | 13 | short stem | 2 | nd |
| 1.3-9 | At5g49760 | 5 | small leaves/ | 4 | short hypocotyl |
| | | | short stem | | on-sucrose |
| | | | | | media in dark |
| 1.5-1 | At2g11520 | 4 | none | 2 | short roots on |
| | | | | | MS |
| 1.5-2 | At1g16260 | 10 | none | 3 | none |
| 1.5-3 | At1g16130 | 6 | none | 3 | short roots on- |
| | | | | | sucrose media |
| 1.5-5 | At1g16110 | 8 | small round | 3 | nd |
| | | | leaves/short | | |
| | | | petiole | | |
| 1.5-11 | At2g23450 | 20 | senescent | 5 | short roots on |
| | | | leave | | MS, short roots |
| | | | | | on-sucrose & - |
| | | | | | nitrogen media |
| | | | | | and under low |
| | | | | | light |
| 1.5-13 | At1g18390 | 20 | none | 3 | nd |
| 1.5-14 | At5g38210 | 2 | none | 1 | nd |
| 1.6-2 | At1g55200 | 20 | none | 2 | nd |
| 1.6-13 | At1g26150 | 5 | none | 2 | nd |
| 1.6-14 | At1g66150 | 19 | apically | 2 | variable |
| | | | dominant | | |
| 1.7-10 | At1g70520 | 3 | late | 2 | late flowering/ |
| | | | flowering/ | | short stem |
| | | | short stem | | |
| 1.7-13 | At4g23140 | 11 | none | 1 | none |
| 1.7-14 | At4g23150 | 5 | none | 2 | none |
| 1.7-19 | At4g23290 | 20 | none | 4 | short roots on |
| | | | | | MS, short roots |
| | | | | | on-sucrose |
| | | | | | media/more |
| | | | | | lateral roots |
| | | | | | on MS* |
| 1.7-21 | At4g11480 | 1 | none | 1 | none |
| 1.7-25 | At4g04570 | 8 | none | 6 | longer roots on |
| | | | | | MS, -nitrogen & |
| | | | | | sorbitol/more |
| | | | | | lateral roots |
| | | | | | on MS* |
| 1.7-29 | At1g61380 | 5 | none | 3 | longer roots/ |
| | | | | | more lateral |
| | | | | | roots on MS* |
| 1.7-31 | At1g11410 | 7 | none | 2 | nd |
| 1.7-34 | At1g61610 | 1 | none | 1 | nd |
| 1.9-1 | At5g38990 | 1 | late | 1 | late flowering/ |
| | | | flowering/ | | large leaves/ |
| | | | large leaves/ | | more leaves/ |
| | | | more leaves/ | | thick stem |
| | | | thick stem | | |
| 1.9-7 | At1g34300 | 9 | late | 2 | late flowering/ |
| | | | flowering/ | | large leaves/ |
| | | | large leaves/ | | more leaves/ |
| | | | more leaves/ | | thick stem |
| | | | thick stem | | |
| 1.9-8 | At4g32300 | 1 | late | 1 | nd |
| | | | flowering/ | | |
| | | | more leaves | | |
| 1.10-1 | At1g21590 | 1 | none | 1 | long roots |
| | | | | | MS/branched |
| | | | | | root |
| | | | | | hairs*/short |
| | | | | | roots on- |
| | | | | | sucrose media |
| 1.11-3 | At5g03140 | 6 | none | 4 | long roots MS/ |
| | | | | | short roots on- |
| | | | | | sucrose media |
| 1.11-5 | At2g37710 | 6 | none | 1 | short roots on |
| | | | | | MS |
| 1.11-10 | At5g60300 | 4 | none | 3 | nd |
| 1.11-11 | At5g01540 | 10 | none | 3 | nd |
| 1.12-3 | At1g69270 | 15 | none | 3 | longer roots on |
| | | | | | MS |
| 1.12-5 | At3g28450 | 5 | none | 4 | short roots on |
| | | | | | MS and -sucrose |
| | | | | | media/bulbous |
| | | | | | root hairs* |
| 1.12-6 | At3g51740 | 7 | none | 7 | longer roots on |
| | | | | | MS, -sucrose |
| | | | | | and 6% sucrose |
| 1.12-8 | At1g12460 | 8 | none | 5 | none |
| 1.12-12 | At5g56040 | 18 | none | 8 | none |
| 1.12-13 | At1g73080 | 11 | none | 4 | longer roots on |
| | | | | | MS, -sucrose |
| | | | | | and -nitrogen |
| | | | | | media |
| 1.12-19 | At5g65700 | 10 | none | 3 | longer roots on |
| | | | | | MS |
| 1.12-21 | At3g47570 | 15 | none | 4 | none |
| 1.12-23 | At5g01890 | 7 | none | 4 | bulbous root |
| | | | | | hairs* |
| 1.12-26 | At3g25560 | 16 | none | 2 | short hypocotyl |
| | | | | | on-sucrose |
| | | | | | media in dark |
| 1.12-27 | At1g71830 | 7 | none | 3 | longer roots on |
| | | | | | MS |
| 1.12-28 | At5g10290 | 12 | none | 2 | longer roots on |
| | | | | | MS/branching |
| | | | | | root hairs* |
| 1.12-29 | At5g63710 | 15 | none | 8 | none |
| 1.12-30 | At5g62710 | 16 | large leaves/ | 8 | root growth |
| | | | thick stem/ | | effected on MS, |
| | | | longer stems | | short hypocotyl |
| | | | | | on-sucrose |
| | | | | | media in dark |
| 1.13-2 | At5g67200 | 4 | none | 2 | root hair |
| | | | | | phenotype*/ |
| | | | | | reduction in |
| | | | | | pavement cell |
| | | | | | lobe number/ |
| | | | | | longer roots on |
| | | | | | MS |
| 1.13-3 | At3g08680 | 8 | none | 6 | none |
| 1.13-4 | At3g02880 | 15 | none | 3 | long roots on |
| | | | | | MS |
| 1.13-5 | At4g23740 | 7 | none | 2 | wavy root hair |
| | | | | | phenotype* |
| 1.13-9 | At3g57830 | 5 | none | 3 | short roots on- |
| | | | | | sucrose media |
| 1.14-5 | At1g78940 | 6 | late | 3 | long roots on |
| | | | flowering/ | | MS |
| | | | long | | |
| | | | petioles/ | | |
| | | | dark green | | |
| | | | leaves | | |
| 1.14-7 | At5g26150 | 6 | none | 3 | none |
| 1.14-10 | At2g45910 | 5 | none | 2 | root hair |
| | | | | | phenotype* |
| 1.15-3 | At5g58540 | 15 | none | 5 | none |
| 1.15-4 | At3g03770 | 4 | none | 3 | none |
| 1.15-5 | At4g39270 | 5 | none | 5 | short roots on- |
| | | | | | sucrose media |
| 1.15-6 | At5g41180 | 3 | none | 1 | nd |
| No Fam-6 | At1g29750 | 1 | short thick | 1 | nd |
| | | | stem | | |
| No Fam-7 | At2g24230 | 12 | none | 6 | none |
| No Fam-9 | At1g66920 | 8 | none | 3 | nd |

*Root hair phenotypes examined by Ornusa Khamsuk

TABLE 3
DN-RLK constructs generated for this project from original cDNA
and confirmed using DNA sequencing.

| | DN-RLK |
|---|---|
| RLK | Constructs |
| Subfamily | (AGI) |
| 1.Other-9 | At4g20790 |
| 1.Other-10 | At5g39390 |
| 1.Other-13 | At2g46850 |
| 1.1-2 | At3g21630 |
| 1.1-6 | At5g06820 |
| 1.3-4 | At5g59700 |
| 1.3-5 | At2g21480 |
| 1.5-2 | At1g16260 |
| 1.5-13 | At1g18390 |
| 1.6-13 | At1g26150 |
| 1.7-14 | At4g23150 |
| 1.7-21 | At4g11480 |
| 1.7-31 | At1g11410 |
| 1.7-34 | At1g61610 |
| 1.14-7 | At5g26150 |
| 1.15-3 | At5g58540 |
| No Fam-9 | At1g66920 |

As used herein, the terms “host cells” and “recombinant host cells” are used interchangeably and refer to cells (for example, an Arabidposis sp., or other plant cell) into which the compositions of the presently disclosed subject matter, for example, an expression vector comprising a dominant negative RLK can be introduced. Furthermore, the terms refer not only to the particular plant cell into which an expression construct is initially introduced, but also to the progeny or potential progeny of such a cell. Because certain modifications can occur in succeeding generations due to either mutation or environmental influences, such progeny might not, in fact, be identical to the parent cell, but are still included within the scope of the term as used herein.

As used herein, the terms “complementarity” and “complementary” refer to a nucleic acid that can form one or more hydrogen bonds with another nucleic acid sequence by either traditional Watson-Crick or other non-traditional types of interactions. In reference to the nucleic molecules of the presently disclosed subject matter, the binding free energy for a nucleic acid molecule with its complementary sequence is sufficient to allow the relevant function of the nucleic acid to proceed, in some embodiments, ribonuclease activity. Determination of binding free energies for nucleic acid molecules is well known in the art. See e.g., Freier et al., 1986; Turner et al., 1987.

A “dominant negative RLK” refers to a polypeptide variant of a native RLK sequence whose expression interferes with or otherwise counteracts native RLK activity. Dominant negative RLK mutants can include a fragment of a RLK polypeptide sequence with at least one mutation. Exemplary mutations include, e.g., RLK polypeptide lacking a functional domain. In other embodiment, the RLK comprises a transmembrane domain but lacks either a kinase domain or a ligand binding domain. In some embodiments, the dominant negative RLK comprise a polypeptide at least 50%, 60%, 70%, 80%, or 90% identical to a wild-type RLK.

As used herein, the phrase “percent complementarity” refers to the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). The terms “100% complementary”, “fully complementary”, and “perfectly complementary” indicate that all of the contiguous residues of a nucleic acid sequence can hydrogen bond with the same number of contiguous residues in a second nucleic acid sequence.

As used herein, the term “gene” refers to a nucleic acid sequence that encodes an RNA, for example, nucleic acid sequences including, but not limited to, structural genes encoding a polypeptide. The term “gene” also refers broadly to any segment of DNA associated with a biological function. As such, the term “gene” encompasses sequences including but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation from one or more existing sequences.

As is understood in the art, a gene typically comprises a coding strand and a non-coding strand. As used herein, the terms “coding strand” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

The phrase “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA syntheses, processing, and transport, as well as polypeptide synthesis, transport, and post-translational modification of polypeptides. Additionally, processes that affect protein-protein interactions within the cell can also affect gene expression as defined herein.

However, in the case of genes that do not encode protein products, for example nucleic acid sequences that encode RNAs or precursors thereof that induce RNAi, the term “gene expression” refers to the processes by which the RNA is produced from the nucleic acid sequence. Typically, this process is referred to as transcription, although unlike the transcription of protein-coding genes, the transcription products of an RNAi-inducing RNA (or a precursor thereof are not translated to produce a protein. Nonetheless, the production of a mature RNAi-inducing RNA from an RNAi-inducing RNA precursor nucleic acid sequence is encompassed by the term “gene expression” as that term is used herein.

The terms “heterologous gene”, “heterologous DNA sequence”, “heterologous nucleotide sequence”, “exogenous nucleic acid molecule”, “exogenous DNA segment”, and “transgene” as used herein refer to a sequence that originates from a source foreign to an intended host cell or, if from the same source, is modified from its original form. Thus, a heterologous gene in a host cell includes a gene that is endogenous to the particular host cell but has been modified, for example by mutagenesis or by isolation from native transcriptional regulatory sequences. The terms also include non-naturally occurring multiple copies of a naturally occurring nucleotide sequence. Thus, the terms refer to a DNA segment that is foreign or heterologous to the cell, or homologous to the cell but in a position within the host cell nucleic acid wherein the element is not ordinarily found.

As used herein, the term “isolated” refers to a molecule substantially free of other nucleic acids, proteins, lipids, carbohydrates, and/or other materials with which it is normally associated, such association being either in cellular material or in a synthesis medium. Thus, the term “isolated nucleic acid” refers to a ribonucleic acid molecule or a deoxyribonucleic acid molecule (for example, a genomic DNA, cDNA, mRNA, RNAi-inducing RNA or a precursor thereof, etc.) of natural or synthetic origin or some combination thereof, which (1) is not associated with the cell in which the “isolated nucleic acid” is found in nature, or (2) is operatively linked to a polynucleotide to which it is not linked in nature. Similarly, the term “isolated polypeptide” refers to a polypeptide, in some embodiments prepared from recombinant DNA or RNA, or of synthetic origin, or some combination thereof, which (1) is not associated with proteins that it is normally found with in nature, (2) is isolated from the cell in which it normally occurs, (3) is isolated free of other proteins from the same cellular source, (4) is expressed by a cell from a different species, or (5) does not occur in nature.

The term “isolated”, when used in the context of an “isolated cell”, refers to a cell that has been removed from its natural environment, for example, as a part of an organ, tissue, or organism.

As used herein, the term “modulate” refers to an increase, decrease, or other alteration of any, or all, chemical and biological activities or properties of a biochemical entity, e.g., a wild type or mutant nucleic acid molecule. For example, the term “modulate” can refer to a change in the expression level of a gene or a level of an RNA molecule or equivalent RNA molecules encoding one or more proteins or protein subunits; or to an activity of one or more proteins or protein subunits that is upregulated or downregulated, such that expression, level, or activity is greater than or less than that observed in the absence of the modulator. For example, the term “modulate” can mean “inhibit” or “suppress”, but the use of the word “modulate” is not limited to this definition.

The term “naturally occurring”, as applied to an object, refers to the fact that an object can be found in nature. For example, a polypeptide or polynucleotide sequence that is present in an organism (including bacteria) that can be isolated from a source in nature and which has not been intentionally modified by man in the laboratory is naturally occurring. It must be understood, however, that any manipulation by the hand of man can render a “naturally occurring” object an “isolated” object as that term is used herein.

As used herein, the terms “nucleic acid”, “nucleic acid molecule” and polynucleotide refer to any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. Nucleic acids can be composed of monomers that are naturally occurring nucleotides (such as deoxyribonucleotides and ribonucleotides), or analogs of naturally occurring nucleotides (e.g., alpha-enantiomeric forms of naturally occurring nucleotides), or a combination of both. Modified nucleotides can have modifications in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid” also includes so-called “peptide nucleic acids”, which comprise naturally occurring or modified nucleic acid bases attached to a polyamide backbone. Nucleic acids can be either single stranded or double stranded.

The terms “operably linked” and “operatively linked” are used interchangeably. When describing the relationship between two nucleic acid regions, each term refers to a juxtaposition wherein the regions are in a relationship permitting them to function in their intended manner. For example, a control sequence “operably linked” to a coding sequence can be ligated in such a way that expression of the coding sequence is achieved under conditions compatible with the control sequences, such as when the appropriate molecules (e.g., inducers and polymerases) are bound to the control or regulatory sequence(s). Thus, in some embodiments, the phrase “operably linked” refers to a promoter connected to a coding sequence in such a way that the transcription of that coding sequence is controlled and regulated by that promoter. Techniques for operably linking a promoter to a coding sequence are well known in the art; the precise orientation and location relative to a coding sequence of interest is dependent, inter alia, upon the specific nature of the promoter.

Thus, the term “operably linked” can refer to a promoter region that is connected to a nucleotide sequence in such a way that the transcription of that nucleotide sequence is controlled and regulated by that promoter region. Similarly, a nucleotide sequence is said to be under the “transcriptional control” of a promoter to which it is operably linked. Techniques for operably linking a promoter region to a nucleotide sequence are known in the art. In some embodiments, a nucleotide sequence comprises a coding sequence and/or an open reading frame. The term “operably linked” can also refer to a transcription termination sequence that is connected to a nucleotide sequence in such a way that termination of transcription of that nucleotide sequence is controlled by that transcription termination sequence.

The term “operably linked” can also refer to a transcription termination sequence that is connected to a nucleotide sequence in such a way that termination of transcription of that nucleotide sequence is controlled by that transcription termination sequence.

In some embodiments, more than one of these elements can be operably linked in a single molecule. Thus, in some embodiments multiple terminators, coding sequences, and promoters can be operably linked together. Techniques are known to one of ordinary skill in the art that would allow for the generation of nucleic acid molecules that comprise different combinations of coding sequences and/or regulatory elements that would function to allow for the expression of one or more nucleic acid sequences in a cell.

The phrases “percent identity” and “percent identical,” in the context of two nucleic acid or protein sequences, refer to two or more sequences or subsequences that have in some embodiments at least 60%, in some embodiments at least 70%, in some embodiments at least 80%, in some embodiments at least 85%, in some embodiments at least 90%, in some embodiments at least 95%, in some embodiments at least 98%, and in some embodiments at least 99% nucleotide or amino acid residue identity, when compared and aligned for maximum correspondence, as measured using one of the following sequence comparison algorithms or by visual inspection. The percent identity exists in some embodiments over a region of the sequences that is at least about 50 residues in length, in some embodiments over a region of at least about 100 residues, and in some embodiments the percent identity exists over at least about 150 residues. In some embodiments, the percent identity exists over the entire length of a given region, such as a coding region.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, for example, by the local homology algorithm described in Smith & Waterman, 1981, by the homology alignment algorithm described in Needleman & Wunsch, 1970, by the search for similarity method described in Pearson & Lipman, 1988, by computerized implementations of these algorithms (GAP, BESTFIT, FASTA, and TFASTA in the GCG WISCONSIN PACKAGE, available from Accelrys, Inc., San Diego, Calif., United States of America), or by visual inspection. See generally, Ausubel et al., 1989.

One example of an algorithm that is suitable for determining percent sequence identity and sequence similarity is the BLAST algorithm, which is described in Altschul et al., 1990. Software for performing BLAST analyses is publicly available through the National Center for Biotechnology Information via the World Wide Web. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences. See e.g., Karlin & Altschul 1993. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

As used herein, the terms “polypeptide”, “protein”, and “peptide”, which are used interchangeably herein, refer to a polymer of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide”, and “peptide” are used interchangeably herein when referring to a gene product. The term “polypeptide” encompasses proteins of all functions, including enzymes. Thus, exemplary polypeptides include gene products, naturally occurring proteins, homologs, orthologs, paralogs, fragments, and other equivalents, variants and analogs of the foregoing.

The terms “polypeptide fragment” or “fragment”, when used in reference to a reference polypeptide, refers to a polypeptide in which amino acid residues are deleted as compared to the reference polypeptide itself, but where the remaining amino acid sequence is usually identical to the corresponding positions in the reference polypeptide. Such deletions can occur at the amino-terminus or carboxy-terminus of the reference polypeptide, or alternatively both. Fragments typically are at least 5, 6, 8, or 10 amino acids long, at least 14 amino acids long, at least 20, 30, 40, or 50 amino acids long, at least 75 amino acids long, or at least 100, 150, 200, 300, 500, or more amino acids long. A fragment can retain one or more of the biological activities of the reference polypeptide. Further, fragments can include a sub-fragment of a specific region, which sub-fragment retains a function of the region from which it is derived.

As used herein, the term “primer” refers to a sequence comprising in some embodiments two or more deoxyribonucleotides or ribonucleotides, in some embodiments more than three, in some embodiments more than eight, and in some embodiments at least about 20 nucleotides of an exonic or intronic region. Such oligonucleotides are in some embodiments between ten and thirty bases in length.

The term “promoter” or “promoter region” each refers to a nucleotide sequence within a gene that is positioned 5′ to a coding sequence and functions to direct transcription of the coding sequence. The promoter region comprises a transcriptional start site, and can additionally include one or more transcriptional regulatory elements. In some embodiments, a method of the presently disclosed subject matter employs a RNA polymerase III promoter.

A “minimal promoter” is a nucleotide sequence that has the minimal elements required to enable basal level transcription to occur. As such, minimal promoters are not complete promoters but rather are subsequences of promoters that are capable of directing a basal level of transcription of a reporter construct in an experimental system. Minimal promoters are often augmented with one or more transcriptional regulatory elements to influence the transcription of an operatively linked gene. For example, cell-type-specific or tissue-specific transcriptional regulatory elements can be added to minimal promoters to create recombinant promoters that direct transcription of an operatively linked nucleotide sequence in a cell-type-specific or tissue-specific manner.

Different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell. As such, promoters are often classified as “constitutive”, “tissue-specific”, “cell-type-specific”, or “inducible”, depending on their functional activities in vivo or in vitro. For example, a constitutive promoter is one that is capable of directing transcription of a gene in a variety of cell types (in some embodiments, in all cell types) of an organism. “Tissue-specific” or “cell-type-specific” promoters, on the other hand, direct transcription in some tissues or cell types of an organism but are inactive in some or all others tissues or cell types. Exemplary tissue-specific promoters include those promoters described in more detail hereinbelow, as well as other tissue-specific and cell-type specific promoters known to those of skill in the art. In some embodiments, a tissue-specific promoter is a seed-specific promoter, leaf specific, root specific promoter.

When used in the context of a promoter, the term “linked” as used herein refers to a physical proximity of promoter elements such that they function together to direct transcription of an operatively linked nucleotide sequence

The term “transcriptional regulatory sequence” or “transcriptional regulatory element”, as used herein, each refers to a nucleotide sequence within the promoter region that enables responsiveness to a regulatory transcription factor. Responsiveness can encompass a decrease or an increase in transcriptional output and is mediated by binding of the transcription factor to the DNA molecule comprising the transcriptional regulatory element. In some embodiments, a transcriptional regulatory sequence is a transcription termination sequence, alternatively referred to herein as a transcription termination signal.

The term “transcription factor” generally refers to a protein that modulates gene expression by interaction with the transcriptional regulatory element and cellular components for transcription, including RNA Polymerase, Transcription Associated Factors (TAFs), chromatin-remodeling proteins, and any other relevant protein that impacts gene transcription.

The term “purified” refers to an object species that is the predominant species present (i.e., on a molar basis it is more abundant than any other individual species in the composition).

A “reference sequence” is a defined sequence used as a basis for a sequence comparison. A reference sequence can be a subset of a larger sequence, for example, as a segment of a full-length nucleotide, or amino acid sequence, or can comprise a complete sequence. Generally, when used to refer to a nucleotide sequence, a reference sequence is at least 200, 300, or 400 nucleotides in length, frequently at least 600 nucleotides in length, and often at least 800 nucleotides in length. Because two proteins can each (1) comprise a sequence (i.e., a portion of the complete protein sequence) that is similar between the two proteins, and (2) can further comprise a sequence that is divergent between the two proteins, sequence comparisons between two (or more) proteins are typically performed by comparing sequences of the two proteins over a “comparison window” (defined hereinabove) to identify and compare local regions of sequence similarity.

The term “regulatory sequence” is a generic term used throughout the specification to refer to polynucleotide sequences, such as initiation signals, enhancers, regulators, promoters, and termination sequences, which are necessary or desirable to affect the expression of coding and non-coding sequences to which they are operatively linked. Exemplary regulatory sequences are described in Goeddel, 1990, and include, for example, the early and late promoters of simian virus 40 (SV40), adenovirus or cytomegalovirus immediate early promoter, the lac system, the trp system, the TAC or TRC system, T7 promoter whose expression is directed by T7 RNA polymerase, the major operator and promoter regions of phage lambda, the control regions for fd coat protein, the promoter for 3-phosphoglycerate kinase or other glycolytic enzymes, the promoters of acid phosphatase, e.g., Pho5, the promoters of the yeast a-mating factors, the polyhedron promoter of the baculovirus system and other sequences known to control the expression of genes of prokaryotic or eukaryotic cells or their viruses, and various combinations thereof. The nature and use of such control sequences can differ depending upon the host organism. In prokaryotes, such regulatory sequences generally include promoter, ribosomal binding site, and transcription termination sequences. The term “regulatory sequence” is intended to include, at a minimum, components the presence of which can influence expression, and can also include additional components the presence of which is advantageous, for example, leader sequences and fusion partner sequences.

In some embodiments, transcription of a polynucleotide sequence is under the control of a promoter sequence (or other regulatory sequence) that controls the expression of the polynucleotide in a cell-type in which expression is intended. It will also be understood that the polynucleotide can be under the control of regulatory sequences that are the same or different from those sequences which control expression of the naturally occurring form of the polynucleotide. As used herein, the phrase “functional derivative” refers to a subsequence of a promoter or other regulatory element that has substantially the same activity as the full length sequence from which it was derived. As such, a “functional derivative” of a seed-specific promoter can itself function as a seed-specific promoter.

Termination of transcription of a polynucleotide sequence is typically regulated by an operatively linked transcription termination sequence (for example, an RNA polymerase III termination sequence). In certain instances, transcriptional terminators are also responsible for correct mRNA polyadenylation. The 3′ non-transcribed regulatory DNA sequence includes in some embodiments about 50 to about 1,000, and in some embodiments about 100 to about 1,000, nucleotide base pairs and contains plant transcriptional and translational termination sequences. Appropriate transcriptional terminators and those that are known to function in plants include the cauliflower mosaic virus (CaMV) 35S terminator, the tml terminator, the nopaline synthase terminator, the pea rbcS E9 terminator, the terminator for the T7 transcript from the octopine synthase gene of Agrobacterium tumefaciens, and the 3′end of the protease inhibitor I or II genes from potato or tomato, although other 3′ elements known to those of skill in the art can also be employed. Alternatively, a gamma coixin, oleosin 3, or other terminator from the genus Coix can be used.

As used herein, the term “RNA” refers to a molecule comprising at least one ribonucleotide residue. By “ribonucleotide” is meant a nucleotide with a hydroxyl group at the 2′ position of a beta-D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, such as to the end(s) of an RNA molecule or internally, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

As used herein, the phrase “double stranded RNA” refers to an RNA molecule at least a part of which is in Watson-Crick base pairing forming a duplex. As such, the term is to be understood to encompass an RNA molecule that is either fully or only partially double stranded. Exemplary double stranded RNAs include, but are not limited to molecules comprising at least two distinct RNA strands that are either partially or fully duplexed by intermolecular hybridization. Additionally, the term is intended to include a single RNA molecule that by intramolecular hybridization can form a double stranded region (for example, a hairpin). Thus, as used herein the phrases “intermolecular hybridization” and “intramolecular hybridization” refer to double stranded molecules for which the nucleotides involved in the duplex formation are present on different molecules or the same molecule, respectively.

As used herein, the phrase “double stranded region” refers to any region of a nucleic acid molecule that is in a double stranded conformation via hydrogen bonding between the nucleotides including, but not limited to hydrogen bonding between cytosine and guanosine, adenosine and thymidine, adenosine and uracil, and any other nucleic acid duplex as would be understood by one of ordinary skill in the art. The length of the double stranded region can vary from about 15 consecutive basepairs to several thousand basepairs. In some embodiments, the double stranded region is at least 15 basepairs, in some embodiments between 15 and 50 basepairs, in some embodiments between 50 and 100 basepairs, in some embodiments between 100 and 500 basepairs, in some embodiments between 500 and 1000 basepairs, and in some embodiments is at least 1000 basepairs. As describe hereinabove, the formation of the double stranded region results from the hybridization of complementary RNA strands (for example, a sense strand and an antisense strand), either via an intermolecular hybridization (i.e., involving 2 or more distinct RNA molecules) or via an intramolecular hybridization, the latter of which can occur when a single RNA molecule contains self-complementary regions that are capable of hybridizing to each other on the same RNA molecule. These self-complementary regions are typically separated by a stretch of nucleotides such that the intramolecular hybridization event forms what is referred to in the art as a “hairpin” or a “stem-loop structure”. In some embodiments, the stretch of nucleotides between the self-complementary regions comprises an intron that is excised from the nucleic acid molecule by RNA processing in the cell.

As used herein, “significance” or “significant” relates to a statistical analysis of the probability that there is a non-random association between two or more entities. To determine whether or not a relationship is “significant” or has “significance”, statistical manipulations of the data can be performed to calculate a probability, expressed as a “P-value”. Those P-values that fall below a user-defined cutoff point are regarded as significant. In some embodiments, a P-value less than or equal to 0.05, in some embodiments less than 0.01, in some embodiments less than 0.005, and in some embodiments less than 0.001, are regarded as significant.

An exemplary nucleotide sequence employed for hybridization studies or assays includes probe sequences that are complementary to or mimic in some embodiments at least an about 14 to 40 nucleotide sequence of a nucleic acid molecule of the presently disclosed subject matter. In one example, probes comprise 14 to 20 nucleotides, or even longer where desired, such as 30, 40, 50, 60, 100, 200, 300, or 500 nucleotides or up to the full length of a given gene. Such fragments can be readily prepared by, for example, directly synthesizing the fragment by chemical synthesis, by application of nucleic acid amplification technology, or by introducing selected sequences into recombinant vectors for recombinant production. The phrase “hybridizing specifically to” refers to the binding, duplexing, or hybridizing of a molecule only to a particular nucleotide sequence under stringent conditions when that sequence is present in a complex nucleic acid mixture (e.g., total cellular DNA or RNA).

As used herein, the term “transcription” refers to a cellular process involving the interaction of an RNA polymerase with a gene that directs the expression as RNA of the structural information present in the coding sequences of the gene. The process includes, but is not limited to, the following steps: (a) the transcription initiation; (b) transcript elongation; (c) transcript splicing; (d) transcript capping; (e) transcript termination; (f) transcript polyadenylation; (g) nuclear export of the transcript; (h) transcript editing; and (i) stabilizing the transcript.

The term “transfection” refers to the introduction of a nucleic acid, e.g., an expression vector, into a recipient cell, which in certain instances involves nucleic acid-mediated gene transfer. The term “transformation” refers to a process in which a cell's genotype is changed as a result of the cellular uptake of exogenous nucleic acid. For example, a transformed cell can express a recombinant form of a polypeptide of the presently disclosed subject matter.

The transformation of a cell with an exogenous nucleic acid (for example, an expression vector) can be characterized as transient or stable. As used herein, the term “stable” refers to a state of persistence that is of a longer duration than that which would be understood in the art as “transient”. These terms can be used both in the context of the transformation of cells (for example, a stable transformation), or for the expression of a transgene (for example, the stable expression of a vector-encoded nucleic acid sequence comprising a trigger sequence) in a transgenic cell. In some embodiments, a stable transformation results in the incorporation of the exogenous nucleic acid molecule (for example, an expression vector) into the genome of the transformed cell. As a result, when the cell divides, the vector DNA is replicated along with plant genome so that progeny cells also contain the exogenous DNA in their genomes.

In some embodiments, the term “stable expression” relates to expression of a nucleic acid molecule (for example, a vector-encoded nucleic acid sequence comprising a trigger sequence) over time. Thus, stable expression requires that the cell into which the exogenous DNA is introduced express the encoded nucleic acid at a consistent level over time. Additionally, stable expression can occur over the course of generations. When the expressing cell divides, at least a fraction of the resulting daughter cells can also express the encoded nucleic acid, and at about the same level. It should be understood that it is not necessary that every cell derived from the cell into which the vector was originally introduced express the nucleic acid molecule of interest. Rather, particularly in the context of a whole plant, the term “stable expression” requires only that the nucleic acid molecule of interest be stably expressed in tissue(s) and/or location(s) of the plant in which expression is desired. In some embodiments, stable expression of an exogenous nucleic acid is achieved by the integration of the nucleic acid into the genome of the host cell.

The term “vector” refers to a nucleic acid capable of transporting another nucleic acid to which it has been linked. One type of vector that can be used in accord with the presently disclosed subject matter is an Agrobacterium binary vector, i.e., a nucleic acid capable of integrating the nucleic acid sequence of interest into the host cell (for example, a plant cell) genome. Other vectors include those capable of autonomous replication and expression of nucleic acids to which they are linked. Vectors capable of directing the expression of genes to which they are operatively linked are referred to herein as “expression vectors”. In general, expression vectors of utility in recombinant DNA techniques are often in the form of plasmids. In the present specification, “plasmid” and “vector” are used interchangeably as the plasmid is the most commonly used form of vector. However, the presently disclosed subject matter is intended to include such other forms of expression vectors which serve equivalent functions and which become known in the art subsequently hereto.

The term “expression vector” as used herein refers to a DNA sequence capable of directing expression of a particular nucleotide sequence in an appropriate host cell, comprising a promoter operatively linked to the nucleotide sequence of interest which is operatively linked to transcription termination sequences. It also typically comprises sequences required for proper translation of the nucleotide sequence. The construct comprising the nucleotide sequence of interest can be chimeric. The construct can also be one that is naturally occurring but has been obtained in a recombinant form useful for heterologous expression. The nucleotide sequence of interest, including any additional sequences designed to effect proper expression of the nucleotide sequences, can also be referred to as an “expression cassette”.

Embodiments of the presently disclosed subject matter provide an expression cassette comprising one or more elements operably linked in an isolated nucleic acid. In some embodiments, the expression cassette comprises one or more operably linked promoters, coding sequences, and/or promoters.

Further encompassed within the presently disclosed subject matter are recombinant vectors comprising an expression cassette according to the embodiments of the presently disclosed subject matter. Also encompassed are plant cells comprising expression cassettes according to the present disclosure, and plants comprising these plant cells.

In some embodiments, the expression cassette is expressed in a specific location or tissue of a plant. In some embodiments, the location or tissue includes, but is not limited to, epidermis, root, vascular tissue, meristem, cambium, cortex, pith, leaf, flower, seed, and combinations thereof.

Embodiments of the presently disclosed subject matter also relate to an expression vector comprising an expression cassette as disclosed herein. In some embodiments, the expression vector comprises one or more elements including, but not limited to, a promoter sequence, an enhancer sequence, a selection marker sequence, a trigger sequence, an intron-containing hairpin transformation construct, an origin of replication, and combinations thereof.

The method comprises in some embodiments introducing into a plant cell an expression cassette comprising a nucleic acid molecule encoding a DN-RLK of the to obtain a transformed plant cell or tissue (also referred to herein as a “transgenic” plant cell or tissue), and culturing the transformed plant cell or tissue. The nucleic acid molecule can be under the regulation of a constitutive or inducible promoter, and in some embodiments can be under the regulation of a tissue—or cell type-specific promoter.

A plant or plant part comprising a cassette encoding a DN-RLK can be analyzed and selected using methods known to those skilled in the art including, but not limited to, Southern blotting, DNA sequencing, and/or PCR analysis using primers specific to the nucleic acid molecule, morphological changes and detecting amplicons produced therefrom.

Coding sequences intended for expression in transgenic plants can be first assembled in expression cassettes operably linked to a suitable promoter expressible in plants. The expression cassettes can also comprise any further sequences required or selected for the expression of the transgene. Such sequences include, but are not limited to, transcription terminators, extraneous sequences to enhance expression such as introns, vital sequences, and sequences intended for the targeting of the transgene-encoded product to specific organelles and cell compartments. These expression cassettes can then be easily transferred to the plant transformation vectors disclosed below. The following is a description of various components of typical expression cassettes.

The selection of the promoter used in expression cassettes can determine the spatial and temporal expression pattern of the transgene in the transgenic plant. Selected promoters can express transgenes in specific cell types (such as leaf epidermal cells, mesophyll cells, root cortex cells) or in specific tissues or organs (roots, leaves, flowers, or seeds, for example) and the selection can reflect the desired location for accumulation of the transgene. Alternatively, the selected promoter can drive expression of the gene under various inducing conditions. Promoters vary in their strength; i.e., their abilities to promote transcription. Depending upon the host cell system utilized, any one of a number of suitable promoters can be used, including the gene's native promoter. The following are non-limiting examples of promoters that can be used in expression cassettes.

Ubiquitin is a gene product known to accumulate in many cell types and its promoter has been cloned from several species for use in transgenic plants (e.g. sunflower-Binet et al., 1991; maize-Christensen & Quail, 1989; and Arabidposis-Callis et al., 1990). The Arabidposis ubiquitin promoter is suitable for use with the nucleotide sequences of the presently disclosed subject matter. The ubiquitin promoter is suitable for gene expression in transgenic plants, both monocotyledons and dicotyledons. Suitable vectors are derivatives of pAHC25 or any of the transformation vectors disclosed herein, modified by the introduction of the appropriate ubiquitin promoter and/or intron sequences.

Several isoforms of actin are known to be expressed in most cell types and consequently the actin promoter can be used as a constitutive promoter. In particular, the promoter from the rice Actl gene has been cloned and characterized (McElroy et al., 1990). A 1.3 kilobase (kb) fragment of the promoter was found to contain all the regulatory elements required for expression in rice protoplasts. Furthermore, expression vectors based on the Acti promoter have been constructed (McElroy et al., 1991). These incorporate the Actl-intron 1, Adhl 5′ flanking sequence (from the maize alcohol dehydrogenase gene) and Adhl-intron 1 and sequence from the CaMV 35S promoter. Vectors showing highest expression were fusions of 35S and Actl intron or the Actl 5′ flanking sequence and the Actl intron. Optimization of sequences around the initiating ATG (of the beta-glucuronidase (GUS) reporter gene) also enhanced expression.

The promoter expression cassettes disclosed in McElroy et al., 1991, can be easily modified for gene expression. For example, promoter-containing fragments are removed from the McElroy constructions and used to replace the double 35S promoter in pCGN1761ENX, which is then available for the insertion of specific gene sequences. The fusion genes thus constructed can then be transferred to appropriate transformation vectors. In a separate report, the rice Actl promoter with its first intron has also been found to direct high expression in cultured barley cells (Chibbar et al., 1993).

A promoter inducible by certain alcohols or ketones, such as ethanol, can also be used to confer inducible expression of a coding sequence of the presently disclosed subject matter. Such a promoter is for example the alcA gene promoter from Aspergillus nidulans (Caddick et a., 1998). In A. nidulans, the alcA gene encodes alcohol dehydrogenase I, the expression of which is regulated by the AlcR transcription factors in presence of the chemical inducer. For the purposes of the presently disclosed subject matter, the CAT coding sequences in plasmid palcA:CAT comprising a alcA gene promoter sequence fused to a minimal 35S promoter (Caddick et al., 1998) are replaced by a coding sequence of the presently disclosed subject matter to form an expression cassette having the coding sequence under the control of the alcA gene promoter. This is carried out using methods known in the art.

Induction of expression of a nucleic acid sequence of the presently disclosed subject matter using systems based on steroid hormones is also provided. For example, a glucocorticoid-mediated induction system can be used and gene expression is induced by application of a glucocorticoid, for example, a synthetic glucocorticoid, for example dexamethasone, at a concentration ranging in some embodiments from 0.1 mM to 1 mM, and in some embodiments from 10 mM to 100 mM.

Another pattern of gene expression is root expression. A suitable root promoter is the promoter of the maize metallothionein-like (MTL) gene disclosed in de Framond, 1991, and also in U.S. Pat. No. 5,466,785, each of which is incorporated herein by reference. This “MTL” promoter is transferred to a suitable vector such as pCGN 1761 ENX for the insertion of a selected gene and subsequent transfer of the entire promoter-gene-terminator cassette to a transformation vector of interest.

Wound-inducible promoters can also be suitable for gene expression. Numerous such promoters have been disclosed (e.g. Xu et al., 1993; Logemann et al., 1989; Rohrmeier & Lehle, 1993; Firek et al., 1993; Warner et al., 1993) and all are suitable for use with the presently disclosed subject matter. Logemann et al. describe the 5′ upstream sequences of the dicotyledonous potato wunl gene. Xu et al. show that a wound-inducible promoter from the dicotyledon potato (pin2) is active in the monocotyledon rice. Further, Rohrmeier & Lehle describe the cloning of the maize Wipl cDNA that is wound induced and which can be used to isolate the cognate promoter using standard techniques. Similarly, Firek et al. and Warner et al. have disclosed a wound-induced gene from the monocotyledon Asparagus officinalis, which is expressed at local wound and pathogen invasion sites. Using cloning techniques well known in the art, these promoters can be transferred to suitable vectors, fused to the genes pertaining to the presently disclosed subject matter, and used to express these genes at the sites of plant wounding.

A maize gene encoding phosphoenol carboxylase (PEPC) has been disclosed by Hudspeth and Grula, 1989. Using standard molecular biological techniques, the promoter for this gene can be used to drive the expression of any gene in a leaf-specific manner in transgenic plants.

A variety of transcriptional terminators are available for use in expression cassettes. These are responsible for termination of transcription and correct mRNA polyadenylation. Appropriate transcriptional terminators are those that are known to function in plants and include the CaMV 35S terminator, the tml terminator, the nopaline synthase terminator, the octopine synthase terminator, and the pea rbcS E9 terminator. These can be used in both monocotyledons and dicotyledons. In addition, a gene's native transcription terminator can be used.

Numerous sequences have been found to enhance gene expression from within the transcriptional unit and these sequences can be used in conjunction with the genes of the presently disclosed subject matter to increase their expression in transgenic plants.

Various intron sequences have been shown to enhance expression, particularly in monocotyledonous cells. For example, the introns of the maize Adhl gene have been found to significantly enhance the expression of the wild type gene under its cognate promoter when introduced into maize cells. Intron 1 was found to be particularly effective and enhanced expression in fusion constructs with the chloramphenicol acetyltransferase gene (Callis et al., 1987). In the same experimental system, the intron from the maize bronzel gene had a similar effect in enhancing expression. Intron sequences have been routinely incorporated into plant transformation vectors, typically within the non-translated leader.

A number of non-translated leader sequences derived from viruses are also known to enhance expression, and these are particularly effective in dicotyledonous cells. Specifically, leader sequences from Tobacco Mosaic Virus (TMV; the “W-sequence”), Maize Chlorotic Mottle Virus (MCMV), and Alfalfa Mosaic Virus (AMV) have been shown to be effective in enhancing expression (see e.g., Gallie et al., 1987; Skuzeski et al., 1990). Other leader sequences known in the art include, but are not limited to, picornavirus leaders, for example, EMCV (encephalomyocarditis virus) leader (5′ noncoding region; see Elroy-Stein et al., 1989); potyvirus leaders, for example, from Tobacco Etch Virus (TEV; see Allison et al., 1986); Maize Dwarf Mosaic Virus (MDMV; see Kong & Steinbiss 1998); human immunoglobulin heavy-chain binding polypeptide (BiP) leader (Macejak & Sarnow, 1991); untranslated leader from the coat polypeptide mRNA of alfalfa mosaic virus (AMV; RNA 4; see Jobling & Gehrke, 1987); tobacco mosaic virus (TMV) leader (Gallie et al., 1989); and Maize Chlorotic Mottle Virus (MCMV) leader (Lommel et al., 1991). See also Della-Cioppa et al., 1987.

Numerous transformation vectors available for plant transformation are known to those of ordinary skill in the plant transformation art, and the genes pertinent to the presently disclosed subject matter can be used in conjunction with any such vectors. The selection of vector will depend upon the selected transformation technique and the target species for transformation. For certain target species, different antibiotic or herbicide selection markers might be employed. Selection markers used routinely in transformation include the nptil gene, which confers resistance to kanamycin and related antibiotics (Messing & Vieira, 1982; Bevan et al., 1983); the bargene, which confers resistance to the herbicide phosphinothricin (White et al., 1990; Spencer et al., 1990); the hph gene, which confers resistance to the antibiotic hygromycin (Blochinger & Diggelmann, 1984); the dhfr gene, which confers resistance to methotrexate (Bourouis & Jarry, 1983); the EPSP synthase gene, which confers resistance to glyphosate (U.S. Pat. Nos. 4,940,935 and 5,188,642); and the mannose-6-phosphate isomerase gene, which provides the ability to metabolize mannose (U.S. Pat. Nos. 5,767,378 and 5,994,629).

Many vectors are available for transformation using Agrobacterium tumefaciens. These typically carry at least one T-DNA border sequence and include vectors such as PBIN19 (Bevan, 1984). Below, the construction of two typical vectors suitable for Agrobacterium transformation is disclosed.

Transformation without the use of Agrobacterium tumefaciens circumvents the requirement for T-DNA sequences in the chosen transformation vector, and consequently vectors lacking these sequences can be utilized in addition to other vectors that contain T-DNA sequences. Transformation techniques that do not rely on Agrobacterium include transformation via particle bombardment, protoplast uptake (e.g. polyethylene glycol (PEG) and electroporation), and microinjection. The choice of vector depends largely on the species being transformed.

Once a DN-RLK is obtained and has been cloned into an expression system, it is transformed into a plant cell. The expression cassettes of the presently disclosed subject matter can be introduced into the plant cell in a number of art-recognized ways. Methods for regeneration of plants are also well known in the art. For example, Ti plasmid vectors have been utilized for the delivery of foreign DNA, as well as direct DNA uptake, liposomes, electroporation, microinjection, and microprojectiles. In addition, bacteria from the genus Agrobacterium can be utilized to transform plant cells. Below are descriptions of representative techniques for transforming both dicotyledonous and monocotyledonous plants, as well as a representative plastid transformation technique.

Transformation techniques for dicotyledons are well known in the art and include Agrobacterium-based techniques and techniques that do not require Agrobacterium. Non-Agrobacterium techniques involve the uptake of exogenous genetic material directly by protoplasts or cells. This can be accomplished by PEG or electroporation-mediated uptake, particle bombardment-mediated delivery, or microinjection. Examples of these techniques are disclosed in Paszkowski et al., 1984; Potrykus et al., 1985; and Klein et al., 1987. In each case the transformed cells are regenerated to whole plants using standard techniques known in the art.

Agrobacterium-mediated transformation is a useful technique for transformation of dicotyledons because of its high efficiency of transformation and its broad utility with many different species. Agrobacterium transformation typically involves the transfer of a binary vector carrying the foreign DNA of interest to an appropriate Agrobacterium strain which can depend on the complement of vir genes carried by the host Agrobacterium strain either on a co-resident Ti plasmid or chromosomally.

Transformation of the target plant species by recombinant Agrobacterium usually involves co-cultivation of the Agrobacterium with explants from the plant and follows protocols well known in the art. Transformed tissue is regenerated on selectable medium carrying the antibiotic or herbicide resistance marker present between the binary plasmid T-DNA borders.

Another approach to transforming plant cells with a gene involves propelling inert or biologically active particles at plant tissues and cells. This technique is disclosed in U.S. Pat. Nos. 4,945,050; 5,036,006; and 5,100,792; all to Sanford et al. Generally, this procedure involves propelling inert or biologically active particles at the cells under conditions effective to penetrate the outer surface of the cell and afford incorporation within the interior thereof. When inert particles are utilized, the vector can be introduced into the cell by coating the particles with the vector containing the desired gene. Alternatively, the target cell can be surrounded by the vector so that the vector is carried into the cell by the wake of the particle. Biologically active particles (e.g., dried yeast cells, dried bacterium, or a bacteriophage, each containing DNA sought to be introduced) can also be propelled into plant cell tissue.

The following examples are provided to further illustrate but not limit the disclosure.

### Examples

One of the major obstacles to studying the function of receptor-like kinases (RLKs) was that in many cases there are many genes in a subfamily and there was the potential for functional redundancy among subfamily members. This redundancy can explain why few RLK genes have been identified using forward genetics-based mutant screens as well as making it difficult to investigate RLK using gene knockout-based reverse genetics. The disclosure provides a novel approach to circumvent this functional redundancy. The approach uses the similarity of the extracellular domains among subfamily members as a way to disrupt the function of the entire subfamily group.

In plants the mechanisms for monitoring the nutrient status is critical for plant growth, development, and responses to the environment. Such mechanisms are presumably linked to nutrient uptake, mobilization and redistribution to regulate plant vegetative growth and reproductive development and growth. However, little was known about the molecular basis of nutrient sensing mechanisms in plants.

Bioinformatics of the Receptor-like Kinase Family in Arabidposis Sequence Annotation, Alignment, and Phylogenetic Analysis. Arabidposis receptor-like kinase gene information was taken from three databases: The Arabidposis Information Resource (TAIR) (www.arabidopsis.org), PlantsP (plantsp.genomics.purdue.edu) and Shiu and Bleecker's 2001 PNAS paper that totaled 651 putative RLKs. Alignment was made using sequences with and without the predicted kinase domain. Because of the interest in extracellular domain homology the methods concentrated on the kinase deletion alignment for further analysis.

Plants used in this project were Arabidposis thaliana ecotype Columbia-0 (Col-0). Before plating, seeds were surface sterilized. First, the seeds were washed in 95% ethanol for 10 minutes, which was removed then the sterilization solution was added (20% bleach, 0.05% tween-20 (Sigma) and double distilled water) and shaken for 10 minutes. The sterilization solution was removed and the seeds were washed three times with sterile distilled water. The seeds were then cold treated for 4 days at 4° C. after plating them on the plates. Four different growth media were prepared for these experiments. For the control conditions: one-half strength Murashige and Skoog (MS) salts (Sigma), 0.5% sucrose (Sigma), 0.8% phyto agar (Research Products International Corp.), 1× B₅ (1,000× in double distilled water: 10% myo-inositol, 0.1% nicotinic acid and 0.1% pyroxidine HCl) and 1× Thiamin (2,000× in double distilled water: 0.2% thiamin HCl). For low nitrogen media: 10× MS micronutrient media (Sigma) was diluted to 0.5× and 10× MS macronutrient containing no nitrogen (40 mM CaCl₂.2H₂O, 30 mM MgSO₄.7H₂O and 12.5 mM KH₂PO₄) was also diluted to 0.5× and 100× Fe.EDTA (18.3 mM FeSO₄ and 12.5 mM EDTA) was also added to a final concentration of 1×. All the other components of the control media were kept the same. For sucrose-less media all components of the control media were included except for the omission of sucrose. All media was brought to pH 5.8 with 1N KOH and autoclaved for 20 minutes. Plates were arranged vertically in the growth room and grown at 22° C. with 150 μM photons/m⁻¹s⁻¹ with a 16 h light, 8 h dark photoperiod.

The Invitrogen Gateway technology was used to expedite the generation of the different RLK mutations used in this study. Generally, a RIKEN cDNA clone (55 RIKEN clones) or wild type seedling cDNA (17 generated by, Table 2.3) was used as a template for polymerase chain reaction (PCR) amplification of the dominant negative. The PCR product was then gel eluted using the Qiagen QIAquick gel extraction kit using the manufacturer's protocol. Eluted DNA was subsequently ligated into Promega's pGEM-Teasy PCR vector. Positive colonies were picked and those with insertions of the DN-RLK into the pGEM vector were confirmed by DNA sequencing: using the T7 and S6 primer sites on the pGEM vector. Confirmed DN-RLK inserts were then restriction digested using the PCR introduced restriction sites (usually SaIl or NotI). The restriction digest was run on a 1% agarose (Invitrogen) gel and the digested insert was removed using the QIAquick kit. The fragment was then ligated into a TAP tagged entry vector that was made by taking the pENTR-1A vector (Invitrogen) and introducing a 6× His and T7 epitope DNA sequence into the EcoRV restriction site in the pENTR-1A vector. This vector was designated pENTR-TAP2. The 3′ ends of all PCR fragments were designed to go into frame with the TAP sequence. The pENTR-TAP2 vectors containing the desired fragments were then introduced into the final destination binary vector that contains the cauliflower mosaic virus (CaMV) 35S promoter, pGWB2 (Invitrogen, Nakagawa). This construct was introduced into Arabidposis (Col-0) via the floral dip method (Bechtold et al., 1993). Subsequent generations of the seeds were selected for using 50 μg/ml Kanamycin (Sigma) in MS media and then transferred to soil until seed set. This process was carried out for subsequent generations until T₃ homozygous lines were found and these lines were used for all of the following experiments. For each construct a minimum of 5 independent lines was generated, but in a few cases less then this was achieved.

Dominant Negative (DN)-RLK plants were examined at all stages of growth for morphological phenotypes. Beginning in the T₁ generation plants were examined when grown on soil and compared to wild type (Col-0) plants for changes in flowering time, leaf size and phyllotaxic aberrations. These phenotypes were recorded and examined in further generations. If the phenotype persisted until the homozygous lines were isolated these phenotypes would then be more carefully examined.

RNA was collected from 10-day old vertically grown seedlings using Qiagen's RNeasy Kit following the manufacture's protocol. Three micrograms of total RNA was used in a reverse transcriptase (Superscript II, Invitrogen) reaction in a 20 μl reaction volume. cDNA obtained from DN-RLK lines was then amplified using gene specific primes and compared to the wild type plants and actin 2 (ACT2) was used as an amplification control.

Examination of Carbon, Nitrogen and Light Requirements of DN-RLKs. Many DN-RLK lines did not show any apparent phenotypes when grown on soil under normal growing conditions, possible RLK functions were examined using nutritional and light screening methods. Because of the many DN-RLK lines that needed to be screened a vertical plate based growth system was used. For examining the responses to sucrose plates containing 0%, 0.5% (normal) and 3-6% sucrose plates were used and root growth examined. For nitrogen requirements DN-RLK lines were grown on 0 mM and 40 mM nitrogen plates and again root growth was examined. Sucrose and light requirements were also examined using 0% and 0.5% sucrose plates grown in the dark and hypocotyl lengths were examined.

The approached provided herein was used to identify potential nutrient sensing molecules from the superfamily, receptor-like kinases. As a proof of concept, Arabidposis dominant negative-RLK transgenic lines were screened on a MS agar medium lacking sucrose and identified four RLK genes that affect sucrose sensing. These results suggest that RLKs play an important role in the regulation of sugar status in plants most likely through its potential role in sensing sugar.

A number of embodiments of the invention have been described. Nevertheless, it will be understood that various modifications may be made without departing from the spirit and scope of the invention. Accordingly, other embodiments are within the scope of the following claims.

What is claimed is:

1. A method of identifying receptor-like kinases (RLKs) that modulate plant function and morphology comprising:
identifying a family of RLKs that comprise at least 50% sequence identity in the extracellular and transmembrane domains;
using a set of PCR primer pair, generating from a cDNA library of RLKs a plurality of RLKs lacking a functional kinase domain (DN-RLKs);
cloning the DN-RLKs into a plant species to obtain recombinant plants comprising at least one DN-RLK from the plurality of DN-RLKs;
expressing the DN-RLKs; and
identifying recombinant plants having morphological or functional traits different than a wild-type plant species.

2. The method of claim 1, wherein the family of RLKs has at least 60%, 70%, 80%, 90%, 95%, 98%, or 99% identity between members of the family.

3. The method of claim 1, wherein the PCR primer pair comprise a first primer comprises a sequence corresponding to the extracellular domain end of the coding sequence and the second primer comprises a sequence that truncates the kinase domain or induces a mutation in the kinase domain that results in a domain lacks kinase activity.

4. The method of claim 1, wherein the plant species is Arabidposis.

5. A plant generated by the method of claim 1.

6. The recombinant plant of claim 5, wherein the plant comprises improved growth characteristics, pathogen resistance, plant height or metabolic activity compared to a wild-type plant.

7. A method of generating a transgene comprising a dominant-negative receptor-like kinases (RLKs) that modulate plant function and morphology comprising:
identifying a family of RLKs that comprise at least 50% sequence identity in the extracellular and transmembrane domains;
using a set of PCR primer pair, generating from a cDNA library of RLKs a plurality of RLKs lacking a functional kinase domain (DN-RLKs);
cloning at least one DN-RLK from the plurality of DN-RLKs into a vector.

8. A method for modulating plant height, organ shape, metabolism, growth characteristics or pathogen resistance comprising the step of expressing a transgene of claim 7 in a plant, wherein the transgene encodes a receptor-like kinase (RLK) protein lacking an active receptor domain or kinase domain and wherein expression of the transgene modulates plant height, organ shape, metabolism, growth characteristics or pathogen resistance.

9. The method of claim 1, 5, 7 or 8, wherein the plant species is a crop plant.

10. A method for enhancing the plant height, organ shape, metabolism, growth characteristics or pathogen resistance of a plant, comprising the steps of: (a) introducing a transgene of claim 7 into a plant, wherein the transgene encodes a receptor-like kinase protein lacking an active receptor domain or kinase domain and wherein expression of the transgene enhances the plant height, organ shape, metabolism, growth characteristics or pathogen resistance of the crop plant; and
(b) growing the transgenic plant under conditions in which the transgene is expressed to enhance the plant height, organ shape, metabolism, growth characteristics or pathogen resistance of the plant.

11. A library of dominant-negative RLK-encoding polynucleotides wherein the polynucleotide encodes a dominant-negative RLK lacking a receptor domain or kinase domain, the library obtained by the method of claim 7.

12. A method of making a library of dominant-negative RLK encoding polynucleotides comprising:
(a) identifying a family of RLKs having at least 50% identity to one another;
(b) mutating the RLKs having identity to disrupt function ligand binding function or kinase function; and
(c) cloning the mutant RLKs.

13. The method of claim 12, further comprising transforming plant cells with the mutant RLKs.

14. The method of claim 13, further comprising growing the mutant cells and identifying cells displaying a mutant phenotype.

15. A library of dominant negative plant cells comprising a transgene encoding a receptor-like kinase lacking a receptor domain or a kinase domain.
